SEQUENCE LISTING (28 sequences)

SEQ ID NO: 1 - protein, 536 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNQARGSGSGRSLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHH

SEQ ID NO: 2 - protein, 536 aa, synthetic construct
MELLILKANAITTILTAVTFCFASGQNITEEFYQSTCSAVSKGYLSALRTGWYTAVITIFLSNIKENKCNGTDAKVKLIKQELDKYKNAVTELQLLMQSTPATNNQARGSGSGRSLGFLLGVGSAIAEGVAVSKVLHLEGEVNKIKSALLSTRKAVVSLSNGVSVLTSKVLDLKNYIDKQLLPIVNKQSCSISNIETVIEFQQKLNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVKLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDNFDASISQVNEKINQSLAFIRKSDELLSAIGGYIPEAPRDGQAYVRKDGEWVLLSTFLGGLVPRGSHHHHHH

SEQ ID NO: 3 - protein, 536 aa, synthetic construct
MELLILKANAITTILTAVTFCFASGQNITEEFYQSTCSAVSKGYLSALRTGWYTVVITIFLSNIKENKCNGTDAKVKLIKQELDKYKNAVTELQLLMQSTPATNNQARGSGSGRSLGFLLGVGSAIALGVAVSKVLHLEGEVNKIKSALLSTRKAVVSLSNGVSVLTSKVLDLKNYIDKQLLPIVNKQSCSISNIETVIEFQQKLNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVKLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDVFDASISQVNEKINQSLAFIRKSDELLSAIGGYIPEAPRDGQAYVRKDGEWVLLSTFLGGLVPRGSHHHHHH

SEQ ID NO: 4 - protein, 536 aa, synthetic construct
MELLILKANAITTILTAVTFCFASGQNITEEFYQSTCSAVSKGYLSALRTGWYTLVITIFLSNIKENKCNGTDAKVKLIKQELDKYKNAVTELQLLMQSTPATNNQARGSGSGRSLGFLLGVGSAIALGVAVSKVLHLEGEVNKIKSALLSTRKAVVSLSNGVSVLTSKVLDLKNYIDKQLLPIVNKQSCSISNIETVIEFQQKFNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVKLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDVFDASISQVNEKINQSLAFIRKSDELLSAIGGYIPEAPRDGQAYVRKDGEWVLLSTFLGGLVPRGSHHHHHH

SEQ ID NO: 5 - protein, 558 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHH

SEQ ID NO: 6 - protein, 558 aa, synthetic construct
MELLILKANAITTILTAVTFCFASGQNITEEFYQSTCSAVSKGYLSALRTGWYTAVITIFLSNIKENKCNGTDAKVKLIKQELDKYKNAVTELQLLMQSTPATNNRARRELPRFMNYTLNNAKKTNVTLSKKRKRRFLGFLLGVGSAIAEGVAVSKVLHLEGEVNKIKSALLSTRKAVVSLSNGVSVLTSKVLDLKNYIDKQLLPIVNKQSCSISNIETVIEFQQKLNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVKLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDNFDASISQVNEKINQSLAFIRKSDELLSAIGGYIPEAPRDGQAYVRKDGEWVLLSTFLGGLVPRGSHHHHHH

SEQ ID NO: 7 - protein, 524 aa, synthetic construct
MSWKVVIIFSLLITPQHGLKESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTGSGYIPEAPRDGQAYVRKDGEWVLLSTFLHHHHHH

SEQ ID NO: 8 - protein, 524 aa, synthetic construct
MSWKVVIIFSLLITPQHGLKESYLEESCSTITEGYLSVLRTDWYTNVFTLEVGDVENLTCADGPSLIKTELDLTKSALRELRTVSADQLNREEQIENPRRRRFVLWFIALGVATAVAVTAGVAIAKTIRDESEVTAIKNALKKTNEAVSTLGNGVRVLLTAVRELKDFVSKNLTRAINKNKCDIPDLKMAVSFSQFNRRFLNVVRQFSENAGITPAISLDLMTDAELARAVSNMPTSAGQIKLMLENRAMVRRKGFGILIGVYGSSVIYMVQLPIFKVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPDKFPPDQFNVALDQVFESIENSQALVDQSNRILSSAEKGNTGSGYIPEAPRDGQAYVRKDGEWVLLSTFLHHHHHH

SEQ ID NO: 9 - protein, 524 aa, synthetic construct
MSWKVVIIFSLLITPQHGLKESYLEESCSTITEGYLSVLRTGWYTNVFTLEVGDVENLTCADGPSLIKTELDLTKSALRELRTVSADQLNREEQIENPRRRRFVLGAIALGVAEAAAVTAGVAIAKTIRDESEVTAIKNALKKTNEAVSTLGNGVRVLLTAVRELKDFVSKNLTRAINKNKCDIPDLKMAVSFSQFNRRFLNIVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKQEGEQHVIKGRPVSSSFDPDKFPEDQFNVALDQVFESIENSQALVDQSNRILSSAEKGNTGSGYIPEAPRDGQAYVRKDGEWVLLSTFLHHHHHH

SEQ ID NO: 10 - protein, 524 aa, synthetic construct
MSWKVVIIFSLLITPQHGLKESYLEESCSTITEGYLSVLRTGWYTNVFTLEVGDVENLTCADGPSLIKTELDLTKSALRELRTVSADQLNREEQIENPRRRRFVLWFIALGVATAVAVTAGVAIAKTIRLESEVTAIKNALKKTNEAVSTLGNGVRVLLTAVRELKDFVSKNLTRAINKNKCDIPDLKMAVSFSQFNRRFLNVVRQFSENAGITPAISLDLMTDAELARAVSNMPTSAGQIKLMLENRAMVRRKGFGILIGVYGSSVIYMVQLPIFEVIDTPCWIVKAAPSCSEKKGNYACLLREDQGWYCQNKGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTGSGYIPEAPRDGQAYVRKDGEWVLLSTFLHHHHHH

SEQ ID NO: 11 - protein, 524 aa, synthetic construct
MSWKVVIIFSLLITPQHGLKESYLEESCSTITEGYLSVLRTGWYTNVFTLEVGDVENLTCADGPSLIKTELDLTKSALRELRTVSADQLNREEQIENPRRRRFVLRAIALGVATAAAVTAGVAIAKTIRDESEVTAIKNALKKTNEAVSTLGNGVRVLITAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFDVIDTPCWIVKAAPSCSEKKGNYACLLREDQGWYCQNKGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPDKFPPDQFNVALDQVFESIENSQALVDQSNRILSSAEKGNTGSGYIPEAPRDGQAYVRKDGEWVLLSTFLHHHHHH

SEQ ID NO: 12 - protein, 524 aa, synthetic construct
MSWKVVIIFSLLITPQHGLKESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKTIRDESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTHAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPIPMVALSPLGALVACYKGVSCSISSNRVGIIKQLNKGCSYITNQDADTVTIDNTVYQLSKVEGEQHVIKGRPVSSSFDPDKFPPDQFNVALDQVFESIENSQALVDQSNRILSSAEKGNTGSGYIPEAPRDGQAYVRKDGEWVLLSTFLHHHHHH

SEQ ID NO: 13 - protein, 524 aa, synthetic construct
MSWKVVIIFSLLITPQHGLKESYLEESCSTITEGYLSVLRTGWYTNVFTLEVGDVENLTCADGPSLIKTELDLTKSALRELRTVSADQLNREEQIENPRRRRFVLFFIALGVATAVAVTAGVAIAKTIRDESEVTAIKNALKKTNEAVSTLGNGVRVLITAVRELKDFVSKNLTRAINKNKCDIPDLKMAVSFSQFNRRFLNVVRQFSENAGITPAISLDLMTDAELARAVSNMPTSAGQIKLMLENRAMVRRKGFGILIGVYGSSVIYMVQLPIFKVIDTPCWIVKAAPSCSEKKGNYACLLREDQGWYCQNKGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPDKFPPDQFNVALDQVFESIENSQALVDQSNRILSSAEKGNTGSGYIPEAPRDGQAYVRKDGEWVLLSTFLHHHHHH

SEQ ID NO: 14 - protein, 524 aa, synthetic construct
MSWKVVIIFSLLITPQHGLKESYLEESCSTITEGYLSVLRTGWYTNVFTLEVGDVENLTCADGPSLIKTELDLTKSALRELRTVSADQLAREEQIENPRRRRFVLWFIALGVAEAAAVTAGVAIAKTIRDESEVTAIKNALKKTNEAVSTLGNGVRVLLTAIRELKDFVSKNLTRAINKNKCDIPDLKMAVSFSQFNRRFLNIVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKQEGEQHVIKGRPVSSSFDPDKFPEDQFNVALDQVFESIENSQALVDQSNRILSSAEKGNTGSGYIPEAPRDGQAYVRKDGEWVLLSTFLHHHHHH

SEQ ID NO: 15 - protein, 524 aa, synthetic construct
MSWKVVIIFSLLITPQHGLKESYLEESCSTITEGYLSVLRTGWYTNVFTLEVGDVENLTCADGPSLIKTELDLTKSALRELRTVSADQLAREEQIENPRRRRFVLGAIALGVAEAAAVTAGVAIAKTIRDESEVTAIKNALKKTNEAVTTLGNGVRVLLTAVRELKDFVSKNLTRAINKNKCDIPDLKMAISFSQFNRRFLNVVRQFSENAGITPRISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKQEGEQHVIKGRPVSSSFDPDKFPEDQFNVALDQVFESIENSQALVDQSNRILSSAEKGNTGSGYIPEAPRDGQAYVRKDGEWVLLSTFLHHHHHH

SEQ ID NO: 16 - protein, 1243 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLHHHHHH

SEQ ID NO: 17 - protein, 1243 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFLGLTVLPPLLTDEMIAQYTSALLAGTITSGWTFGAGAALQIPFQMQMAYRFNGIGVTQNVFWENQKLIANQFNSAIGKIQDSLSSDASALGKLQDVVNQNLQALNTLVEQLSSNFGAISSVLNDILSRLNPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQQELDSFKEELDKYFKNHTSPDVDLGDISGINASVVNIQKEIDRLNEVAKNLNESLIDLQELGKYEQGSGYIPEAPRDGQAYVRKDGEWVLLSTFLHHHHHH

SEQ ID NO: 18 - protein, 1243 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTKIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSDASALGKLQDVVNQNLQALNTLVKQLSSNFGAISSVLNDILSRLDPPEARVQIDRLITGRLQSLQTYVTQQLIRAIEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQQELDSFKEELDKYFKNHTSPDVDLGDISGINASVVNIQKEIDRLNEVAKNLNESLIDLQELGKYEQGSGYIPEAPRDGQAYVRKDGEWVLLSTFLHHHHHH

SEQ ID NO: 19 - protein, 1243 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICTDSTECSNLLLQYGSFCTQLNRALTKIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSDASALGKLQDVVNQDAQALNTLVKQLSSNFGAISSVLNDILSRLNPPEARVQIDRLITGRLQSLQTYVTQQLIRAIEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQNELDSFKEELDKYFKNHTSPDVDLGDISGINASVVNIQKEIDRLNEVAKNLNESLIDLQELGKYEQGSGYIPEAPRDGQAYVRKDGEWVLLSTFLHHHHHH

SEQ ID NO: 20 - protein, 1243 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTKIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVFYENQKLIANQFNSAIGKIQDSLSSNPSALGKLQDVVNQNAQALNTLVKQLSSNFGAISSVLNDILSRLDPPEAEVQIDRLITGRLQSLMTYVIQQQLRAIEIRLSAKLAATKMSECVLGQSKRVDFCGKGYHLMTFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLHHHHHH

SEQ ID NO: 21 - DNA, 1611 bp, synthetic construct
  source 1..1611: mol_type = other DNA
atggagcttcttatcctgaaggccaatgccattaccaccattctgaccgcagtgaccttttgttttgccagcggccaaaacatcaccgaggagttttaccagagtacctgcagcgccgtttccaaaggctacctgagcgccctgaggaccggatggtacaccgccgtcataacaatcttcttgtctaacattaaggagaacaaatgtaacgggaccgacgcaaaggtgaaactgattaagcaggagctggacaaatacaagaatgccgtaaccgagcttcaactgctcatgcagagcactcccgcaactaacaaccaggccagaggcagcggatccggcagaagcctgggcttcctgctgggcgtgggcagcgcaatagccgagggcgttgctgttagcaaagtgctccacctggaaggcgaagtgaacaaaatcaagagcgcactgctgagcacgcgcaaggccgtggtatccctcagcaacggtgtctctgtgctgacatctaaggtgctggacctgaagaactatatcgacaagcaacttctgccaatcgtcaacaagcagagctgttctattagcaacatcgagaccgtgatcgagttccagcagaagctgaataggctgttggagatcacgagagagttctccgtgaatgctggggtaacgacgccggtttccacctacatgctgaccaatagcgaactgcttagcctgatcaacgacatgcccatcaccaacgaccagaaaaagctgatgtcaaacaacgtgcagatcgtgcgccagcaatcttactccatcatgtcaatcataaaggaggaagtgcttgcctacgttgtccaactgccgctgtacggcgtgatagacaccccatgttggaagctgcatactagccccctgtgcactaccaataccaaggagggcagcaatatatgcctgaccaggacagatagggggtggtactgtgacaatgccggtagcgtctccttcttcccgcaggccgagacctgtaaggtacaaagcaaccgcgtattctgcgacacgatgaattccctgacactgcccagcgaggtcaagctgtgcaacgtggatatcttcaaccccaagtacgactgcaagatcatgaccagcaagacagacgttagcagcagcgttatcacaagcctgggcgccatcgtaagctgctacggcaagacgaagtgcaccgcgagcaataagaacaggggcatcatcaagaccttcagtaacggctgcgactacgtgagtaataaaggcgtggacaccgtatccgtcggcaacaccctgtactacgtaaataagcaagaaggcaagagcctgtacgtgaagggagagcctatcattaacttctacgaccccctggtgtttcctagcgataacttcgacgcctctatctcacaggtaaacgagaagataaaccaaagccttgcatttatccgaaagagcgacgagctgctttccgccatcggcggctacatccccgaggcccctagagatggccaggcctacgtgcggaaggacggcgagtgggtgctgctgtccaccttcctgggcggcctggtgcccagaggcagccaccaccatcaccatcactaa

SEQ ID NO: 22 - DNA, 1575 bp, synthetic construct
  source 1..1575: mol_type = other DNA
atgagctggaaggtggtgatcatcttcagcctgctgatcacccctcagcacggcctgaaggagagctacctggaggagagctgcagcaccatcaccgagggctacctgagcgtgctgagaaccggctggtacaccaacgtgttcaccctggaggtgggcgacgtggagaacctgacctgcgccgacggccctagcctgatcaagaccgagctggacctgaccaagagcgccctcagagagttaagaaccgtgagcgccgatcagctgaacagagaggagcagatcgagaaccctagacgcagaagatttgtgctgggcgctattgccctgggcgtggcagaagcggccgcggttaccgccggcgtggccatcgccaagaccatcagagacgagagcgaggtgaccgccatcaagaacgccctgaagaagaccaacgaggccgtgagcaccctgggcaacggcgtgagagtgctgctgaccgccgtacgtgaactgaaggatttcgtgagcaagaacctgacaagagccatcaacaagaacaagtgcgacatccccgacctgaagatggccgtgagcttctctcagttcaacagaagattcctgaacatcgtgagacagttcagcgacaacgccggcatcacccccgccatcagcctggacctgatgaccgacgccgagctggctagagccgtgagcaacatgcccacaagcgccgggcagatcaagctgatgctggagaacagagccatggtgagaagaaagggcttcggcatcctgatcggcgtgtacggcagcagcgtgatctacatggtgcagctgcccatcttcggcgtgatcgacaccccctgctggatcgtgaaggccgcccctagctgcagcgagaagaagggcaactacgcctgcctgctgagagaggaccaaggctggtactgtcagaacgccggcagcaccgtgtactaccccaacgagaaggactgcgagacaagaggcgaccacgtgttctgcgacaccgccgccggcatcaacgtggccgagcagagcaaggagtgcaacatcaacatcagcaccaccaactacccctgcaaggtgagcaccggcagacatccgatcagcatggttgccctatcccccctgggcgccctggtggcctgctacaagggcgtgagctgcagcatcggcagcaacagagtgggcatcatcaagcagctgaacaagggctgcagctacatcaccaaccaagacgccgacaccgtgaccatcgacaacaccgtgtatcagctgagcaagcaagagggcgagcagcacgtgatcaagggcagacccgtgagcagcagcttcgaccccgacaagttccccgaggatcagttcaacgtggccctggaccaagtgttcgagagcatcgagaacagccaagccctggtggatcagagcaacagaatcctgagcagcgccgagaagggcaacaccggcagcggctacatccccgaggcccctagagacggccaagcctacgtgagaaaggacggcgagtgggtgctgctgagcaccttcctgcaccaccaccaccaccactaa

SEQ ID NO: 23 - DNA, 1575 bp, synthetic construct
  source 1..1575: mol_type = other DNA
atgagctggaaggtggtgatcatcttcagcctgctgatcacccctcagcacggcctgaaggagagctacctggaggagagctgcagcaccatcaccgagggctacctgagcgtgctgagaaccggctggtacaccaacgtgttcaccctggaggtgggcgacgtggagaacctgacctgcgccgacggccctagcctgatcaagaccgagctggacctgaccaagagcgccctcagagagctgcgaaccgtgagcgccgatcagctgaacagagaggagcagatcgagaaccctagaagaagacgcttcgtcctgagggcaatcgccctgggggtcgcgaccgcggcggccgtaaccgccggcgtggccatcgccaagaccatcagagacgagagcgaggtgaccgccatcaagaacgccctgaagaagaccaacgaggccgtgagcaccctgggcaacggcgtgagagtgctgatcaccgccgtgagagaactgaaagacttcgtgagcaagaacctgacaagagccatcaacaagaacaagtgcgacatccccgacctgaagatggccgtgagcttctctcagttcaacagaagattcctgaacgtggtgagacagttcagcgacaacgccggcatcacccccgccatcagcctggacctgatgaccgacgccgagctggctagagccgtgagcaacatgcccacaagcgccgggcagatcaagctgatgctggagaacagagccatggtgagaagaaagggcttcggcatcctgatcggcgtgtacggcagcagcgtgatctacatggtgcagctgcccatcttcgacgtgatcgacaccccctgctggatcgtgaaggccgcccctagctgcagcgagaagaagggcaactacgcctgcctgctgagagaggaccaaggctggtactgtcagaacaagggcagcaccgtgtactaccccaacgagaaggactgcgagacaagaggcgaccacgtgttctgcgacaccgccgccggcatcaacgtggccgagcagagcaaggagtgcaacatcaacatcagcaccaccaactacccctgcaaggtgtctaccggcagacaccctataagcatggtggcactgtcgccactgggcgccctggtggcctgctacaagggcgtgagctgcagcatcggcagcaacagagtgggcatcatcaagcagctgaacaagggctgcagctacatcaccaaccaagacgccgacaccgtgaccatcgacaacaccgtgtatcagctgagcaaggtggagggcgagcagcacgtgatcaagggcagacccgtgagcagcagcttcgaccccgacaagttcccccccgatcagttcaacgtggccctggaccaagtgttcgagagcatcgagaacagccaagccctggtggatcagagcaacagaatcctgagcagcgccgagaagggcaacaccggcagcggctacatccccgaggcccctagagacggccaagcctacgtgagaaaggacggcgagtgggtgctgctgagcaccttcctgcaccaccaccaccaccactaa

SEQ ID NO: 24 - DNA, 3732 bp, synthetic construct
  source 1..3732: mol_type = other DNA
atgttcgtgttcctggtgctgctccctctggtgagcagccagtgcgtgaacctgactaccagaacccagctgcctccagcttatacaaatagcttcaccaggggcgtgtactaccccgacaaggtgttcagaagcagcgtgctgcattctacacaggatctgttcctgccattcttcagcaacgttacatggttccacgccatccacgtgtctggcaccaatggcaccaagcggttcgacaaccccgtgctgcctttcaacgacggggtgtatttcgccagcacagagaagtccaacatcatccggggatggatcttcggcaccaccctggacagcaagacacaaagcctgctgatcgtgaacaacgctaccaacgtggtgatcaaagtgtgcgagttccagttctgcaacgacccttttctgggagtgtactaccacaagaacaacaaaagctggatggaaagcgagtttagagtgtacagttctgctaacaactgcacatttgagtacgtgagccaacccttcttgatggacctggagggcaagcagggcaacttcaagaacctcagagagttcgtgttcaagaatatcgatggctacttcaagatttactccaagcacacccccatcaacctggtacgtgatctgccgcagggattctccgccctggaacctctggtggacctgcccatcggcattaacatcacgagattccagactctgctggcacttcacagatcttacctgacacctggcgattccagcagcggatggaccgccggcgctgccgcttattacgtcggctacctgcaacctaggaccttcctgctgaagtacaacgagaacggcactatcaccgacgctgtggattgcgccctggaccctctgtctgagacaaagtgcaccctcaaaagttttacagtggaaaagggcatctaccagaccagtaacttcagagttcagcctaccgaaagcatcgtgcggttccctaacattacaaatctgtgtcccttcggagaagtcttcaacgccaccagattcgcctctgtttacgcctggaaccggaaaagaatctctaactgcgtggccgactactctgtgctgtacaactctgcgtcctttagcacattcaaatgctacggcgtgagccctaccaagctgaatgacttgtgcttcaccaacgtatacgctgacagcttcgtgatcagaggcgatgaggtgagacagatagcccctggccagacaggcaagatcgccgattacaactacaagctgccagacgacttcaccggctgcgtaatcgcctggaactctaacaatctggattcaaaggtgggaggcaactacaactacctgtatagactgttccggaagagcaatctgaagccttttgagagagatatcagcaccgagatctaccaggccggatctaccccatgtaatggcgtggaaggctttaattgctacttcccactgcagagctacggctttcagcctacaaacggcgtcggatatcagccctacagggtggtggtgctgagcttcgaactgctgcatgcacctgccaccgtgtgcggccctaagaagagcacaaacctggtcaagaacaagtgtgtcaactttaacttcaatggcctgacaggcaccggagtgctgacagagagcaacaagaagttcctgccattccagcagttcggcagagacattgccgacaccaccgacgccgtgagagaccctcagaccctggaaatcctggacatcaccccttgttcttttggcggcgtgagcgtgatcacccccggcacaaacacctctaaccaggtggccgtgctgtaccaggacgtgaactgcaccgaggtgcctgtggccatccacgcagaccagctgacaccaacctggcgggtgtacagcacaggcagcaacgtgttccagacacgggccggctgtctgatcggagctgaacacgtgaataatagctacgagtgtgacattcctatcggcgccggcatctgcgccagctaccagacccagacgaactcccctggaagcgcctcatctgtggctagtcagagtatcatcgcctataccatgtctctgggagccgagaactctgttgcctattccaacaactccatcgccatccccaccaacttcaccatcagcgtgacaaccgaaatcctgccggttagcatgaccaagaccagcgttgactgcaccatgtacatctgcggcgacagcactgaatgtagcaacctgttgctgcaatacggcagcttctgtacacagctgaacagagccctgaccggcatcgctgtggagcaggacaagaacacccaggaggtgttcgcccaagtgaaacagatctacaagacccctcccatcaaagattttgggggcttcaactttagccagatcctgcctgaccccagcaaacctagcaaaagaagcttcatcgaggacctgctcttcaacaaggtcaccctggccgatgccggattcatcaagcagtacggcgactgcctgggggatatcgccgctagagacctgatctgcgcccagaaattcctggggctgaccgtgctacctcctctgctgaccgacgagatgatcgcccagtacaccagtgctctgctggccggcactatcacatcgggctggaccttcggagctggagctgctctgcagatccccttccagatgcagatggcctaccggtttaatgggatcggcgtgacccagaatgtcttttgggagaaccagaagctgatcgccaatcagttcaatagcgccatcggcaaaatccaggatagcctgagcagcgacgccagcgccctgggcaagctgcaggatgtcgttaaccagaatctccaggccctgaataccctggtggaacagctttctagcaacttcggcgccatctcaagcgtgctgaatgatatcctgagcagactgaaccctccagaggctgaagtgcagatcgacagacttatcaccggcagactgcagtccctgcaaacctacgtgacgcaacaactgattagagccgccgaaattagagccagcgccaacctggccgccacaaagatgagcgagtgcgtgctgggccagtctaagcgggtggacttctgcggcaaaggctaccacctgatgagcttccctcagagcgcacctcacggcgtggtgttcctgcacgtgacctacgtgcccgcccaggagaagaacttcacaaccgcccctgccatctgccacgacggcaaggcccacttccctcgcgagggcgtgttcgtgtctaatggaacacactggttcgtgacgcagagaaacttctacgaaccccagatcattacaacagataacaccttcgtgtccggtaattgcgacgtggtgatcggcatcgtgaacaacaccgtgtacgaccccctgcagcaggagctggattcattcaaggaagaactggacaagtactttaagaaccacaccagccctgacgtggatctgggcgacatcagcggcatcaacgccagcgtggtgaacatccagaaagagatcgaccggctgaacgaggtcgcaaagaacttaaacgagagcctgatcgacctgcaggaactgggcaagtacgagcaggggagcggatacatccctgaggcccctagagatggtcaagcctatgtgcggaaggacggcgagtgggtgctgctgagcacctttctgcaccaccaccaccaccattaa

SEQ ID NO: 25 - DNA, 3732 bp, synthetic construct
  source 1..3732: mol_type = other DNA
atgttcgtgtttctggttctgctgcctctggtgtcttctcagtgcgtgaacctgaccacaagaacccagctgcctccagcctacaccaattctttcacaagaggcgtgtactaccctgacaaggtgttcagaagcagcgtgctgcacagcacccaagacctgtttctgcccttcttcagcaacgtgacctggttccacgccattcacgtgtctggaaccaacggcaccaagcggttcgacaaccctgtgctgcctttcaacgacggagtgtacttcgccagcaccgagaaaagcaacatcatccggggctggatctttggcacgaccctggacagtaagacccagagcctcctgatcgtgaacaatgccacaaacgtggtgatcaaggtgtgcgagttccagttctgtaacgacccgttcctgggagtgtactaccacaagaacaacaagtcctggatggaatctgaattcagagtgtacagctctgctaacaactgtaccttcgagtacgtgtcccagcctttcctgatggacctggagggcaagcaaggcaacttcaagaacctgagagaattcgtgttcaagaatatcgacgggtattttaaaatctacagcaagcacacccccatcaacctggtgagagatctgcctcaaggcttttctgccctggaacctctagtggacctgccgatcggcatcaacattacacggtttcagacactgctggccctgcatagaagctacctgacacctggcgatagcagctcaggctggaccgccggcgcggccgcctactacgtgggctatctgcagcctagaacattcctgctgaagtacaacgaaaatggcaccatcacagacgctgtggactgcgcccttgatcctctgtctgagacaaagtgcaccctgaagagctttaccgttgagaagggcatctatcagacctctaacttccgggtgcagccaacagaaagcatcgtgcgcttccccaacatcaccaacctgtgtccttttggagaggtctttaacgccacaagattcgcctctgtctacgcctggaacagaaagcggatctccaactgtgtggctgattacagcgtgctgtacaactccgcctccttctccaccttcaagtgctacggagtgtctccaaccaagctgaatgatttgtgcttcaccaacgtgtacgccgatagtttcgtgatcagaggcgatgaggtccggcagatcgcccctggccagactggcaaaatcgctgactacaactacaaactgcccgacgactttacaggctgtgtgatcgcctggaactccaacaacctcgacagcaaagtgggtggcaactacaactacttgtaccggctgttcagaaagagcaacctaaagcctttcgagagagacatcagcacagagatctaccaggccggctcgaccccttgcaatggcgtggaagggttcaactgctacttccccctgcagagctacggcttccaacccaccaacggagtcggctaccagccttaccgggtggtggtactgagcttcgagctgctgcacgcacctgccaccgtatgcggtcctaagaagtctaccaatctggtgaagaacaagtgcgtgaacttcaacttcaacggcctgaccggcacaggcgtcctcactgagagcaacaaaaaattcctgcccttccagcagtttggacgggacatcgctgacaccaccgatgccgtgagggacccccagaccctggaaatcctggacatcaccccttgttctttcggcggcgtgtcagtgatcacccctggcaccaatacaagcaaccaggtcgccgtgctgtaccaggacgtgaattgcacagaggtgcctgtggctatccacgccgaccagctcaccccgacatggcgggtgtacagcaccggaagcaatgtgttccagacccgggctggctgcctgatcggcgctgagcacgtaaacaacagctacgagtgcgacatccccatcggcgccggcatctgcgcctcctaccaaacccagactaacagccccggcagcgccagctctgtggccagccagtccatcatcgcctacaccatgagcctgggcgccgagaacagcgtggcctacagcaataattcaatcgccattcctacaaacttcaccatctctgtgaccacagagatccttcctgtgtccatgaccaagacctccgtggactgcaccatgtacatctgcggcgactctacagaatgcagcaacctgctgctgcagtacggtagcttttgcacccagctgaaccgagccctgaccaagatcgccgttgaacaggacaagaatacccaggaagtatttgcccaggttaagcagatctacaagacaccaccaatcaaggacttcggcggattcaatttcagccaaatcctgccagatcctagcaagcccagcaagcggagcttcatcgaggatctgctgttcaataaggtgaccctggccgacgccggcttcatcaaacagtacggcgactgcctgggagacatcgccgccagggatctgatctgtgcccagaagttcaacggcctgaccgtgctgcctcctctgctgaccgacgagatgatcgcccagtacaccagcgccctgctggccggcacaatcacatctggctggacatttggcgctggtgccgccctgcagatccctttcgccatgcagatggcctatagatttaacggaatcggcgtgacacagaacgttctgtatgaaaaccagaaactaatcgctaatcagttcaactcagccattggaaagatccaggacagcctgtccagcgacgccagcgccctcggcaagctgcaggatgtggtgaaccagaacctccaagccctgaacaccctggtgaagcagctgtctagtaactttggcgctatttctagcgtgctgaatgacatcctgtccagactggaccctcctgaggccagagtgcagatcgacagactgatcacaggcagactgcaaagcctgcaaacctacgtgacccagcagctgatccgggccatcgaaatcagagccagcgccaacctggccgctacaaagatgagcgagtgcgtgctgggccagtctaaaagagtggatttctgcggcaagggctaccacctgatgagcttcccccagagcgctccccacggcgtggtgttcctgcacgtgacatacgtgcccgctcaggaaaaaaatttcaccacggcccccgctatctgccacgacggaaaggcccacttccctagagagggcgtgtttgtgagcaacggcacacactggttcgtgactcaaagaaacttctacgagcctcagattatcaccacagataacacattcgtgtccggaaactgtgacgtagtgatcggcatcgtgaacaatacggtgtacgaccccctccagcaggagctggattctttcaaagaggaactggataagtatttcaagaaccacaccagtccagatgttgatctgggcgacatatctggaatcaacgccagcgtcgtgaacatccaaaaggaaatcgatagactgaacgaggtggcaaagaatttgaacgaaagcctgattgacctgcaagagctgggcaagtacgagcagggcagcggctacatccccgaggcccctcgggatggacaggcttacgtgcgaaaagacggcgagtgggtgctgctgagcaccttcctgcaccatcaccatcaccactaa

SEQ ID NO: 26 - DNA, 3732 bp, synthetic construct
  source 1..3732: mol_type = other DNA
atgttcgtgttcctggtgctactcccgctggtgagcagccagtgcgtgaacctgaccaccaggacccagctgcctcctgcctacaccaatagcttcacccggggcgtgtactaccccgacaaggtgttcagatcttccgtgctgcacagcacccaggatctgttcctgccttttttcagcaacgttacctggttccacgccatccacgtgagcggaaccaacggcaccaagcgcttcgacaaccccgtgctccctttcaacgacggagtgtactttgccagtacagagaagtctaatatcatcagaggctggatcttcggtaccaccctggacagcaagacccagagcctgctgatcgtgaataacgctacaaatgtggtgatcaaagtgtgtgaattccagttctgtaacgaccctttcctgggcgtgtactatcacaagaacaacaaatcttggatggaaagcgaatttagagtgtactctagcgcaaacaactgcaccttcgagtacgtgtcccagcctttcctgatggacctggagggcaagcaaggcaacttcaagaacctgcgggagttcgtattcaaaaacatcgacgggtacttcaaaatctacagtaagcacacccctatcaacctggtgcgggatctgcctcagggctttagcgccctggaacctctggtggacctgcccatcggaattaacattaccagatttcagaccctgctggcactgcacagaagctacctgacaccaggcgacagctccagcggctggaccgccggcgccgctgcttactacgtgggatacctgcaacctcggaccttcctactgaagtacaacgagaatggcaccatcaccgacgccgtggactgcgccctcgaccctctgagtgaaaccaaatgcacactgaagagtttcaccgtggaaaagggcatctaccagaccagtaacttcagagtccagccgacagaaagcatcgtgcggttccccaatatcaccaatctgtgtcctttcggcgaagtgtttaatgccacaagattcgcctctgtgtatgcctggaacagaaaacggatcagcaactgcgtggccgattacagcgtgctgtacaacagcgccagcttcagcacatttaagtgctacggcgtgtccccaacaaagctgaatgacctgtgcttcaccaacgtgtatgccgacagcttcgtgatccggggcgacgaggtgcgacagatcgcccctggccagacaggcaagatcgccgactacaactacaagctgccagatgacttcaccggctgcgtgatagcctggaatagtaacaacctggatagcaaggtgggcggcaactacaactacctatatagattgtttcggaagagcaatctgaaacctttcgagagagacatctctaccgagatctaccaggctggcagcacaccctgtaatggcgtcgagggatttaattgctactttcccctgcagagctacggcttccagcctaccaacggcgtgggataccagccatacagagtggtggtgctgtccttcgagctgctgcatgcccctgccacagtgtgcggccctaagaagagcacgaacctggtgaagaacaagtgcgtgaacttcaactttaacggcctgaccggcaccggcgtgctgaccgaatccaacaagaaattcctgcccttccagcagtttggcagagacatcgccgacaccaccgacgccgtgagggaccctcaaacactggaaatcctggatataacaccttgcagcttcggtggcgtgagcgtgatcacacctggaacgaacaccagcaaccaggtggccgtgctgtaccaggatgtgaactgcaccgaggtgcctgtggccatccacgctgaccagctgacccctacctggcgggtatacagcacaggcagcaacgtgttccaaacaagagccggatgtctgataggcgcagaacacgttaacaacagctacgagtgcgacatccccatcggggccggcatctgtgccagctaccagacccagacaaactctcctggcagcgcatcctccgtcgccagccagagcatcatcgcttataccatgtccctgggcgccgagaactccgtggcctacagcaacaacagcatcgccatccccacaaacttcaccatcagcgtgaccaccgagatcctgcctgtgagcatgaccaagacaagcgtcgattgtacaatgtacatctgcacggattccactgaatgcagcaacctgctcctgcaatacggtagcttctgcacacagctcaatagagccctgacaaagatcgccgtggaacaggataagaatactcaagaggtgttcgcccaagtgaaacagatctataagacaccccctatcaaggacttcggcggattcaacttctctcagatcctgcccgatccgagcaagcccagcaagcggagcttcatcgaggacctgctgttcaacaaggtgaccctggccgatgctggatttatcaagcagtacggcgactgcctgggcgacatcgccgccagagatctgatctgcgcccagaaattcaacggcctgaccgttctgcctcctctactaaccgacgagatgatagcccagtacaccagcgctttgcttgccggcacaatcacctccggctggacgttcggcgccggcgctgctctgcagatccccttcgccatgcagatggcctatagattcaacggaattggagtcacccagaacgtgctgtacgaaaaccaaaaactgatcgccaaccagttcaacagcgcgatcggcaaaatccaggacagcctctcatctgacgcttctgccctgggcaagctgcaggacgtggtgaaccaggacgcccaggccctgaacactctggtcaagcagctgtctagcaattttggagccatttcttctgtgcttaatgatatcctgtcaagactgaaccctccagaggccagagtgcagatcgatagactgatcacaggaagactgcagagcctgcagacctacgtgacacagcagctgatccgggctatcgagatcagagccagcgccaacctggctgccaccaagatgtccgagtgcgtgctgggccagtcaaagagggtggacttttgtggcaaaggctaccacctgatgagctttcctcagtctgctcctcacggcgtcgtgttcctgcatgtcacatacgtgcccgcccaggagaaaaactttaccaccgctcctgccatctgccacgacggcaaggcccacttccctcgggaaggcgtttttgtgtctaacggcacccactggtttgtgacccagcggaacttctacgagcctcagatcatcaccacggataacacattcgtctccggcaattgtgacgtcgtgattggaatcgtgaacaataccgtgtacgacccactgcaaaatgaactggattctttcaaggaagagttggacaagtacttcaagaaccacaccagccctgatgtggatctgggcgacatctctggcattaacgccagcgtggtgaacatccagaaggagatcgacagactgaacgaagtggccaagaatctgaatgagagcctcatcgacctgcaggagctgggcaagtacgagcagggcagcgggtatatccctgaggcccctagagatggccaggcctacgtgcggaaagacggcgagtgggtgctgctgtcgaccttcctgcaccaccaccaccatcactaa

SEQ ID NO: 27 - DNA, 3732 bp, synthetic construct
  source 1..3732: mol_type = other DNA
atgttcgtgttcctcgtgctgctgcctctggttagctctcagtgcgtgaacctgaccacccggacccagctgcctcctgcttataccaatagctttaccagaggcgtatactaccctgacaaggttttccgcagcagcgtgttgcacagcacccaggacctgttcctgcctttcttcagcaacgtgacatggttccacgccatccacgtatctggtacaaacggcaccaagcggttcgacaaccccgtgctgcctttcaacgacggcgtgtacttcgcgagcacagagaagagtaacatcatccggggctggatcttcggcaccaccctggatagcaaaacccagagcctgctgatcgtgaacaacgccacaaacgtggtgatcaaggtatgtgaatttcagttttgcaacgacccttttctgggcgtctactaccacaagaacaacaagagctggatggaaagcgagttccgggtgtattcctccgcaaacaattgcacattcgagtacgtgtcccagcctttcctgatggaccttgagggcaagcagggcaactttaaaaacctcagagaattcgtgttcaaaaacatcgacggctatttcaagatctacagcaagcacacccccatcaacctggtgcgggatctgcctcaaggcttcagcgccctggaacccctggtggacctgcctatcggaatcaacatcaccagattccaaacgctgctggccctgcacagaagctacctgacacctggcgactctagcagcggatggaccgccggtgcagccgcttactacgtgggctacctgcagcctagaaccttcctgctgaaatacaacgaaaacggcacaatcacagacgctgtcgattgcgccctggaccccctgagcgagacaaaatgcaccctgaagtctttcacagtggaaaagggcatctaccagaccagcaactttagagtgcagcctaccgagagcatcgtgcggttcccaaatatcacaaacctgtgtcctttcggcgaagtattcaacgccacccggttcgccagcgtgtacgcctggaatagaaagagaatcagcaactgtgtggcggactactctgtgctgtataatagcgccagcttctctacatttaagtgctacggcgtgagtcctaccaagctgaacgacttgtgcttcaccaacgtgtacgccgacagctttgtgatccggggcgacgaggtgcggcagatcgcccccggccagacaggcaagatcgccgactacaactacaagctgcctgatgacttcaccggctgtgtgattgcctggaacagcaacaacctggacagcaaagtgggcggaaattacaactacctgtacagattgttcagaaagagcaacctgaagcccttcgaaagagatatttcgacagaaatctaccaggccggatccacaccctgtaacggagtggagggtttcaactgctactttcccctgcagtcctacggattccagcccaccaacggcgtcggctaccagccatacagagtggtggtcctctccttcgagctgcttcacgcccctgccactgtgtgcggacctaagaaaagcaccaacctggtgaagaacaaatgcgtgaactttaatttcaacggcctgacgggcacaggcgtgctgaccgagtcaaacaaaaagttcctgccgttccagcaattcggccgtgacatcgccgacaccaccgacgccgtgcgggatcctcaaaccctggaaatcctggacatcacaccatgcagcttcggaggagtgtccgtcatcacccctggcaccaatacgtctaatcaggtggccgtgctgtaccaggatgtgaactgcaccgaggtgccagtggccatccacgccgatcagctgacacccacctggagagtgtacagcaccggatctaatgtgttccagaccagagccggctgcctgatcggcgcggagcacgtgaacaactcatacgaatgcgacatccccatcggcgccggcatctgcgccagctatcagacccagaccaactcccctggaagcgccagtagtgtcgccagccaaagtatcatcgcttataccatgtccctcggcgctgagaacagcgtggcctacagcaacaattctatcgctattcccacaaatttcaccatctctgtgaccaccgaaatcttacctgtgtccatgacaaagaccagcgtggactgcaccatgtacatctgtggcgactccactgagtgcagcaatctgctgctgcaatacggctccttttgcacccaactgaatagagccctgaccaagatcgccgtggaacaggacaagaacacccaggaggtgttcgcccaagtgaagcagatttataagacccctcctatcaaagacttcggcggctttaacttcagccagattctgcccgatcccagcaagccttctaagcggtccttcatcgaggacctgcttttcaacaaggtgaccctggccgatgccgggttcattaagcagtacggcgattgcctgggcgacatcgctgccagagatctgatctgtgcccagaagttcaacggcctgaccgtgcttcccccattgctgacagacgaaatgatcgcccagtacacctctgccctgctggctggcacaatcacatccggctggacattcggcgcaggcgccgccctgcagatcccctttgccatgcagatggcctatagatttaacggcatcggtgttacacagaacgtgttctacgagaaccagaagctgattgctaatcagttcaactctgctattggcaaaatccaggacagcctgagcagcaatccttcagctctgggcaagctgcaagacgtggtgaatcaaaacgcccaggccctgaacaccctggtgaagcaactgtcttctaatttcggcgccatcagctctgtgctgaacgacattctgtctagactggatccacctgaggccgaggtccagatcgacaggcttatcaccggcaggctgcagagcctgatgacctacgtgattcagcagcagctgagagccatcgagatcagactgagcgctaagctggccgccaccaagatgagcgagtgcgtgctgggccagagcaagcgagtggatttctgcggcaagggctaccacctgatgaccttcccccaatctgccccccacggcgtcgtgttcctgcacgtgacatacgtgcccgcacaggagaagaactttacgacagccccagccatctgtcacgacggcaaggcccacttccctagagagggagtgtttgttagcaatggaacccactggttcgtaacccagagaaacttctacgagcctcaaatcatcaccaccgataacaccttcgtgagcggcaactgtgacgtggtgatcggcatcgtgaacaacaccgtgtacgaccctctgcagcctgagctggacagctttaaggaagagctggataagtacttcaagaaccataccagccctgacgtggacctgggagatatcagcggcatcaacgccagcgtggtgaacatccagaaagaaatcgacagactgaacgaagtggccaagaacctgaatgagagcctgatcgatctgcaggagctgggaaagtacgaacagggcagcggatacatccctgaggcccctcgggatggacaggcctatgtgcggaaggacggcgaatgggtgctgctgtccactttcctgcatcaccaccaccatcactaa

SEQ ID NO: 28 - DNA, 12 bp, synthetic construct
  source 1..12: mol_type = other DNA
gctagcgccacc

(19) United States
(12) Patent Application Publication
(10) Pub. No.: US 2024/0226274 A1
(43) Pub. Date: Jul. 11, 2024

(54) RECOMBINANT VIRAL CLASS I FUSION PROTEINS AND USES THEREOF

(71) Applicant: University of Georgia Research Foundation, Inc., Athens, GA (US)

(72) Inventors: Eva-Maria Strauch, St. Louis, MO (US); Karen Juliana Gonzalez Restrepo, Athens, GA (US)

(73) Assignee: University of Georgia Research Foundation, Inc., Athens, GA (US)

(21) Appl. No.: 18/404,463

(22) Filed: Jan. 4, 2024

Related U.S. Application Data
(60) Provisional application No. 63/437,414, filed on Jan. 6, 2023.

(51) Int. Cl. A61K 39/215; A61P 31/14; C07K 14/005

(52) U.S. Cl. CPC A61K 39/215; A61P 31/14; C07K 14/005; C12N 2760/18522; C12N 2760/18534; C12N 2770/20022; C12N 2770/20034

(57) ABSTRACT

Recombinant viral class I fusion proteins that favor a prefusion conformational state over a postfusion conformational state, polynucleotides encoding same, vaccines comprising the proteins or polynucleotides, and methods of vaccination with the vaccines. The recombinant proteins include recombinant RSV F proteins, recombinant hMPV proteins, and recombinant SARS-COV-2 S proteins.

### CROSS-REFERENCE TO RELATED APPLICATIONS

Priority is hereby claimed to U.S. Provisional Application 63/437,414, filed Jan. 6, 2023, which is incorporated herein by reference in its entirety.

### STATEMENT REGARDING FEDERALLY SPONSORED RESEARCH

This invention was made with government support under AI140245 awarded by the NIH. The government has certain rights in the invention. (37 CFR 401.14 f (4))

### SEQUENCE LISTING

The instant application contains a Sequence Listing which has been electronically submitted in XML format and is hereby incorporated by reference in its entirety. The XML copy, created on Jan. 4, 2024, is named USPTO-240104-105677002-2023-038-02-SEQ LIST.xml and is 59,041 bytes in size.

### FIELD OF THE INVENTION

The invention is directed to recombinant viral class I fusion proteins that favor a prefusion conformational state over a postfusion conformational state, polynucleotides encoding same, vaccines comprising the proteins or polynucleotides, and methods of vaccination with the vaccines.

### BACKGROUND

Life-threatening viruses such as the human immunodeficiency virus (HIV)¹, Ebola virus², Pneumoviruses³, and the pandemic influenza⁴ and coronaviruses⁵, use class I fusion proteins to induce the fusion of viral and cellular membranes and infect the host cell. During membrane fusion, class I fusion proteins refold from their metastable conformation (prefusion state) to the highly stable postfusion conformation, likely to provide the energy mediating the fusion reaction⁶. Their essential role in the viral entry and their location on the viral surface makes class I fusion proteins one of the major targets of neutralizing antibodies and, thereby, a critical immunogen for vaccination⁷. However, while both pre- and postfusion states are usually immunogenic, the labile prefusion state has been demonstrated to induce a more potent immune response in multiple viral families⁸⁻¹². Consequently, the prefusion state has become an attractive vaccine candidate when its conformation can be maintained^{8,13,14}.

Recombinant class I fusion proteins that favor the prefusion state are needed.

### SUMMARY OF THE INVENTION

One aspect of the invention is directed to recombinant viral class I fusion proteins.

One embodiment is directed to recombinant RSV F proteins comprising a first RSV F sequence at least 80%, 85%, 90%, 95%, or 99% identical to positions 26-494 of a second RSV F sequence selected from the group consisting of SEQ ID NOS: 1-4 or positions 26-516 of a third RSV F sequence selected from the group consisting of SEQ ID NOS:5-6. The first RSV F sequence preferably comprises 1 or more, 2 or more, 3 or more, 4 or more, 5 or more, 6 or more, 7 or more, 8 or more, 9 or more, 10 or more, or 11 or more of: a residue other than serine at a position corresponding to position 55 of the second or third RSV F sequence; a residue other than glutamate at a position corresponding to position 60 of the second or third RSV F sequence; a residue other than asparagine at a position corresponding to position 70 of the second or third RSV F sequence; a residue other than glutamine at a position corresponding to position 94 of the second or third RSV F sequence; a residue other than methionine at a position corresponding to position 97 of the second or third RSV F sequence; a residue other than serine at a position corresponding to position 128 of the second RSV F sequence or position 150 of the third RSV F sequence; a residue other than glycine at a position corresponding to position 129 of the second RSV F sequence or position 151 of the third RSV F sequence; a residue other than asparagine at a position corresponding to position 153 of the second RSV F sequence or position 175 of the third RSV F sequence; a residue other than glutamine at a position corresponding to position 203 of the second RSV F sequence or position 225 of the third RSV F sequence; a residue other than asparagine at a position corresponding to position 205 of the second RSV F sequence or position 227 of the third RSV F sequence; a residue other than valine at a position corresponding to position 256 of the second RSV F sequence or position 278 of the third RSV F sequence; a residue other than serine at a position corresponding to position 308 of the second RSV F sequence or position 330 of the third RSV F sequence; a residue other than threonine at a position corresponding to position 315 of the second RSV F sequence or position 337 of the third RSV F sequence; a residue other than glutamate at a position corresponding to position 356 of the second RSV F sequence or position 378 of the third RSV F sequence; a residue other than asparagine at a position corresponding to position 358 of the second RSV F sequence or position 380 of the third RSV F sequence; a residue other than glutamate at a position corresponding to position 465 of the second RSV F sequence or position 487 of the third RSV F sequence; cysteines at positions corresponding to positions 52 and 128 of the second RSV F sequence or positions 52 and 150 of the third RSV F sequence; cysteines at positions corresponding to positions 55 and 166 of the second RSV F sequence or positions 55 and 188 of the third RSV F sequence; cysteines at positions corresponding to positions 60 and 174 of the second RSV F sequence or positions 60 and 196 of the third RSV F sequence; cysteines at positions corresponding to positions 135 and 161 of the second RSV F sequence or positions 157 and 183 of the third RSV F sequence; and cysteines at positions corresponding to positions 421 and 444 of the second RSV F sequence or positions 443 and 466 of the third RSV F sequence.

Another embodiment is directed to recombinant hMPV proteins comprising a first hMPV F sequence at least 80%, 85%, 90%, 95%, or 99% identical to positions 19-489 of a second hMPV F sequence selected from the group consisting of SEQ ID NOS:7-15. The first hMPV F sequence preferably comprises 1 or more, 2 or more, 3 or more, 4 or more, 5 or more, 6 or more, 7 or more, 8 or more, 9 or more, 10 or more of, 11 or more, 12 or more, 13 or more, 14 or more, 15 or more, 16 or more, 17 or more, or 18 or more of: a residue other than glycine at a position corresponding to position 42 of the second hMPV F sequence; a residue other than alanine at a position corresponding to position 90 of the second hMPV F sequence; a residue other than glycine at a position corresponding to position 106 of the second hMPV F sequence; a residue other than alanine at a position corresponding to position 107 of the second hMPV F sequence; a residue other than threonine at a position corresponding to position 114 of the second hMPV F sequence; a residue other than alanine at a position corresponding to position 116 of the second hMPV F sequence; a residue other than leucine at a position corresponding to position 130 of the second hMPV F sequence; a residue other than lysine at a position corresponding to position 143 of the second hMPV F sequence; a residue other than serine at a position corresponding to position 149 of the second hMPV F sequence; a residue other than threonine at a position corresponding to position 150 of the second hMPV F sequence; a residue other than glycine at a position corresponding to position 152 of the second hMPV F sequence; a residue other than alanine at a position corresponding to position 159 of the second hMPV F sequence; a residue other than threonine at a position corresponding to position 160 of the second hMPV F sequence; a residue other than valine at a position corresponding to position 162 of the second hMPV F sequence; a residue other than leucine at a position corresponding to position 187 of the second hMPV F sequence; a residue other than valine at a position corresponding to position 191 of the second hMPV F sequence; a residue other than valine at a position corresponding to position 203 of the second hMPV F sequence; a residue other than aspartate at a position corresponding to position 209 of the second hMPV F sequence; a residue other than alanine at a position corresponding to position 216 of the second hMPV F sequence; a residue other than serine at a position corresponding to position 237 of the second hMPV F sequence; a residue other than glycine at a position corresponding to position 277 of the second hMPV F sequence; a residue other than alanine at a position corresponding to position 314 of the second hMPV F sequence; a residue other than serine at a position corresponding to position 371 of the second hMPV F sequence; a residue other than glycine at a position corresponding to position 393 of the second hMPV F sequence; a residue other than valine at a position corresponding to position 430 of the second hMPV F sequence; a residue other than isoleucine at a position corresponding to position 437 of the second hMPV F sequence; a residue other than valine at a position corresponding to position 449 of the second hMPV F sequence; a residue other than glutamate at a position corresponding to position 453 of the second hMPV F sequence; cysteines at positions corresponding to positions 43 and 120 of the second hMPV F sequence; cysteines at positions corresponding to positions 45 and 157 of the second hMPV F sequence; cysteines at positions corresponding to positions 113 and 336 of the second hMPV F sequence; cysteines at positions corresponding to positions 115 and 375 of the second hMPV F sequence; cysteines at positions corresponding to positions 123 and 429 of the second hMPV F sequence; and cysteines at positions corresponding to positions 211 and 253 of the second hMPV F sequence.

Another embodiment is directed to recombinant SARS-COV-2 S proteins comprising a first SARS-COV-2 S sequence at least 80%, 85%, 90%, 95%, or 99% identical to positions 15-1208 of a second SARS-COV-2 S sequence selected from the group consisting of SEQ ID NOS:16-20, wherein: the first SARS-COV-2 S sequence comprises 1 or more, 2 or more, 3 or more, 4 or more, 5 or more, 6 or more, 7 or more, 8 or more, 9 or more, 10 or more, 11 or more, or 12 or more of: a residue other than alanine at a position corresponding to position 706 of the second SARS-COV-2 S sequence; a residue other than threonine at a position corresponding to position 732 of the second SARS-COV-2 S sequence; a residue other than glycine at a position corresponding to position 744 of the second SARS-COV-2 S sequence; a residue other than alanine at a position corresponding to position 766 of the second SARS-CoV-2 S sequence; a residue other than glycine at a position corresponding to position 769 of the second SARS-COV-2 S sequence; a residue other than threonine at a position corresponding to position 827 of the second SARS-COV-2 S sequence; a residue other than isoleucine at a position corresponding to position 844 of the second SARS-COV-2 S sequence; a residue other than arginine at a position corresponding to position 847 of the second SARS-COV-2 S sequence; a residue other than asparagine at a position corresponding to position 856 of the second SARS-COV-2 S sequence; a residue other than alanine at a position corresponding to position 899 of the second SARS-COV-2 S sequence; a residue other than glycine at a position corresponding to position 908 of the second SARS-COV-2 S sequence; a residue other than threonine at a position corresponding to position 912 of the second SARS-COV-2 S sequence; a residue other than leucine at a position corresponding to position 916 of the second SARS-CoV-2 S sequence; a residue other than tyrosine at a position corresponding to position 917 of the second SARS-COV-2 S sequence; a residue other than threonine at a position corresponding to position 941 of the second SARS-COV-2 S sequence; a residue other than alanine at a position corresponding to position 942 of the second SARS-COV-2 S sequence; a residue other than asparagine at a position corresponding to position 955 of the second SARS-COV-2 S sequence; a residue other than alanine at a position corresponding to position 956 of the second SARS-COV-2 S sequence; a residue other than lysine at a position corresponding to position 964 of the second SARS-COV-2 S sequence; a residue other than aspartate at a position corresponding to position 985 of the second SARS-COV-2 S sequence; a residue other than glutamate at a position corresponding to position 990 of the second SARS-COV-2 S sequence; a residue other than threonine at a position corresponding to position 998 of the second SARS-CoV-2 S sequence; a residue other than glutamine at a position corresponding to position 1005 of the second SARS-COV-2 S sequence; a residue other than threonine at a position corresponding to position 1009 of the second SARS-COV-2 S sequence; a residue other than leucine at a position corresponding to position 1012 of the second SARS-COV-2 S sequence; a residue other than isoleucine at a position corresponding to position 1013 of the second SARS-COV-2 S sequence; a residue other than alanine at a position corresponding to position 1016 of the second SARS-COV-2 S sequence; a residue other than alanine at a position corresponding to position 1020 of the second SARS-COV-2 S sequence; a residue other than asparagine at a position corresponding to position 1023 of the second SARS-COV-2 S sequence; a residue other than serine at a position corresponding to position 1051 of the second SARS-COV-2 S sequence; and a residue other than proline at a position corresponding to position 1143 of the second SARS-COV-2 S sequence.

Another aspect of the invention is directed to recombinant polynucleotides encoding recombinant viral class I fusion proteins of the invention.

Another aspect of the invention is directed to vaccines comprising the recombinant viral class I fusion proteins of the invention or recombinant polynucleotides of the invention.

Another aspect of the invention is directed methods of vaccination. The methods preferably comprise administering a vaccine of the invention to a subject in an amount effective to elicit an immune response against a recombinant viral class I fusion protein of the invention.

The objects and advantages of the invention will appear more fully from the following detailed description of the preferred embodiment of the invention made in conjunction with the accompanying drawings.

### BRIEF DESCRIPTION OF THE DRAWINGS

FIG. 1. Computational design overview. Step-by-step diagram illustrating the key components of redesigning viral class I fusion proteins. Both pre- and postfusion conformations are input into the pipeline to identify substitutions that favor the prefusion state over the postfusion conformation. RMSD=Root mean square deviation; ΔΔG=change in delta Gibbs free energy.

FIG. 2. Summary of cryo-electron microscopy data for design Spk-M. Micrographs were processed in cryoSPARC V3.3.2 and final refinement in DeepEM enhancer.

FIG. 3. Gibbs free energy changes (ΔΔG) after alanine scanning in the pre- and postfusion structures of RSV F, hMPV F, and SARS-COV-2 S proteins. Mutations favoring the prefusion state over the postfusion conformation are characterized by positive scores where higher values represent bigger energetic gaps between states. A solid line represents the kernel density estimate of each histogram. On display are presented only significant ΔΔG changes as defined by a differential of at least 2 units.

FIG. 4. Energy comparison between prefusion base constructs and designed variants. In black is represented the prefusion conformation while grey depicts the postfusion conformation. An orange dotted line highlights the energy of the starting sequences in Rosetta Energy Units (REU). To compare the energy gain or loss of each variant, all prefusion energies were normalized to the postfusion state using the ratio postfusion-energy_base_construct/prefusion-energy_base_construct. The energy gap between states is shown in Table 8.

FIGS. 5A-5I. Biochemical characterization of designed variants. (FIG. 5A) Size-exclusion chromatography (SEC) of monodispersed RSV F designs. (FIG. 5B) Binding of design R-1b to the prefusion-specific antibody D25 compared to the clinical candidate DS-Cav1 and the postfusion RSV F A2 (post). (FIG. 5C) Differential scanning fluorimetry (DSF) of design R-1b and the clinical candidate DS-Cav1. DS-Cav1 was used to compare the stability of R-1b as the parental sequence of the latter is not prefusion-stabilized. (FIG. 5D) SEC of monodispersed hMPV F designs. (FIG. 5E) Binding of designed hMPV F variants to the prefusion-specific antibody MPE8 compared to their parent prefusion construct 115B-V and the postfusion hMPV B2 F (post). (FIG. 5F) DSF of designed hMPV F variants and their parent prefusion construct 115B-V. (FIG. 5G) SEC of monodispersed SARS-COV-2 S designs. (FIG. 5H) Binding of designed SARS-COV-2 S variants to ACE2 compared to their parent prefusion construct S-2P. (FIG. 5I) DSF of designed SARS-COV-2 S variants and their parent prefusion construct S-2P. Antibody binding assays show in grey the raw data, in colors the fitted curves, and in dotted lines the end of the association time. Binding constants are shown in Tables 9, 10, and 11.

FIG. 6. Size-exclusion chromatography of design M-104 in comparison to its parent construct 115B-V.

FIG. 7. Binding of RSV F variants to the prefusion-specific antibody AM14. In grey is shown the raw data and in colors the fitted curves. A dotted vertical line represents the end of the association time. Binding constants are shown in Table 9. “Post” stands for postfusion RSV F A2.

FIG. 8. Binding of hMPV F variants to the prefusion-specific antibody 465. In grey is shown the raw data and in colors the fitted curves. A dotted vertical line represents the end of the association time. Binding constants are shown in Table 10. “Post” stands for postfusion hMPV B2 F.

FIG. 9. Binding of design Spk-M and its base construct S-2P to ACE2 after heat treatment. In grey is shown the raw data and in colors the fitted curves. A dotted vertical line represents the end of the association time. Binding constants are shown in Table 11.

FIG. 10. Binding of design M-104 and its base construct 115B-V to prefusion-specific antibodies after heat treatment. (A) Binding to MPE8. (B) Binding to 465. In grey is shown the raw data and in colors the fitted curves. A dotted vertical line represents the end of the association time. Binding constants are shown in Table 10.

FIG. 11. Binding of RSV F variants to prefusion- and postfusion-specific antibodies after heat treatment. (A) Binding to D25. (B)) Binding to AM14. (C) Binding to 131-2A. “Post” stands for postfusion RSV A2 F. As R-1b does not bind to 131-2A after heating at 60° C., only the raw data is displayed for this protein. In grey is shown the raw data and in colors the fitted curves. A dotted vertical line represents the end of the association time. Binding constants are shown in Table 9.

FIG. 12. Negative stain-electron microscopy of designs (A) R-1b, (B) M-104, and (C) Spk-M. Scale bar: 100 nm.

FIG. 13. Exemplary stabilizing substitutions of leading designs. (A) R-1b. (B) M-104. (C) S2 subunit of Spk-M with cryo-EM map. The computational model of each protein is displayed as a trimeric structure in grey while the crystal structures and cryo-EM reconstruction model are displayed as monomeric structures in blue (RSV), magenta (hMPV), or green (SARS-COV-2). Each panel shows a magnified view of selected stabilizing substitutions, featured in yellow sticks, aligned with their computational model. Residues involved in packing changes are displayed with translucent molecular surfaces, and black dotted lines represent hydrogen bonds or salt bridges. As density is missing in the overall map to assign the precise location of the side chains, we displayed existing density as mesh representation to compare agreement with the computational model as stabilized regions have more density than the remainder of the map. The stabilization mechanism of all designed substitutions is presented in Table 8.

FIG. 14. Structural alignment between head residues of different RSV F variants. (A) Comparison between design R-1b (blue) and parent construct PDB 5w23 (grey). (B) Comparison shown in A plus three different DS-Cav1 crystal structures in green (PDB 4mmu, 5ea4, 5k6c). On display are residues 195-227.

FIG. 15. Atomic interactions of all substitutions introduced in design R-1b compared with a computational model. The computational model of the protein is displayed as a trimeric structure in grey, while the crystal structure is displayed as a monomeric structure in blue, with introduced mutations in yellow. Each panel shows a magnified view of the atomic interactions involving each substitution (in yellow sticks), aligned with their computational model. Residues contributing to packing changes are displayed with translucent molecular surfaces, and black dotted lines represent hydrogen bonds or salt bridges.

FIG. 16. Atomic interactions of all substitutions introduced in design M-104 compared with a computational model. The computational model of the protein is displayed as a trimeric structure in grey, while the crystal structure is displayed as a monomeric structure in purple with introduced mutations in yellow. Each panel shows a magnified view of the atomic interactions involving each substitution (in yellow sticks), aligned with their computational model. Residues contributing to packing changes are displayed with translucent molecular surfaces, and black dotted lines represent hydrogen bonds or salt bridges.

FIG. 17. Predicted atomic interactions of all designed substitutions introduced in the S2 subunit of design Spk-M. The computational model of the protein is displayed as a trimeric structure in grey, while the cryo-EM reconstruction model is displayed as a monomeric structure in green with introduced mutations in yellow. The Spk-M cryo-EM map is shown as a translucent surface in grey. Each panel shows a magnified view of the atomic interactions involving each substitution (in yellow sticks), aligned with their computational model. As density is missing in the overall map to assign the precise location of the side chains, we displayed existing density as a mesh representation to compare agreement with the computational model. Black dotted lines represent hydrogen bonds or salt bridges.

FIG. 18. Computational models of postfusion destabilizing substitutions introduced in (A) R-1b. (B) M-104. (C) Spk-M. Each panel shows a magnified view of the predicted rotamer configuration of each mutation. All substitutions are represented in yellow sticks.

FIG. 19. Immunogenicity assessment of R-1b in a mouse model using 0.2 μg doses. (A) Schematic diagram of vaccination study schedule. (B) Serum RSV-specific IgG measured by ELISA three weeks post-boost. (C) Serum RSV-specific IgG measured by ELISA nine weeks post-boost. (D) Serum neutralization titers determined using RSV A (rA2 strain L19F) and sera from mice nine weeks post-boost. Vertical lines represent the standard deviation of three repetitions using pooled serum samples from mice in each immunization group (5 animals/group).

FIG. 20. Immunogenicity assessment of design R-1b in mice using 2 μg doses. A) Serum RSV-specific IgG measured by ELISA three weeks post-boost. (B) Serum RSV-specific IgG measured by ELISA nine weeks post-boost.

### DETAILED DESCRIPTION OF THE INVENTION

One aspect of the invention is directed to recombinant viral class I fusion proteins. The recombinant viral class I fusion proteins of the invention include recombinant RSV F proteins derived from the native respiratory syncytial virus (RSV) F protein, recombinant human metapneumovirus (hMPV) F proteins derived from the native human metapneumovirus (hMPV) F protein, and recombinant SARS COV-2 S proteins derived from the native SARS CoV-2 S protein. The recombinant viral class I fusion proteins of the invention accordingly comprise sequences derived from the corresponding native viral class I fusion proteins. Sequences derived from the RSV F protein are referred to herein as “RSV F” sequences. Sequences derived from the hMPV F protein are referred to herein as “hMPV F” sequences. Sequences derived from the SARS COV-2 S protein are referred to herein as “SARS-COV-2 S” sequences.

The recombinant RSV F proteins of the invention preferably comprise a first RSV F sequence at least 80% identical to positions 26-494 of a second RSV F sequence selected from the group consisting of SEQ ID NOS:1-4 or positions 26-516 of a third RSV F sequence selected from the group consisting of SEQ ID NOS:5-6. SEQ ID NOS: 1-4 are exemplary RSV F constructs described herein (see Table 1 and the following examples). Positions 26-494 of SEQ ID NOS: 1-4 include portions corresponding to residues 1-105 and 137-513 of the RSV F protein (excluding the signal sequence, which is cleaved after expression), and a modified furin cleavage site, (see Table 2 and the following examples). SEQ ID NOS:5-6 are additional exemplary RSV F constructs derived from NCBI Accession No. 5W23_A that include a full, internal native sequence in place of the modified furin cleavage site and p27 peptide of SEQ ID NOS: 1-4. Positions 1-108 and 114-536 of SEQ ID NOS: 1-4 correspond to positions 1-108 and 136-558 of SEQ ID NOS:5-6. Differences between SEQ ID NOS:1-4 at positions 109-113 and SEQ ID NOS:5-6 at positions 109-135 result in a reduction in size of 22 residues in SEQ ID NOS: 1-4 with respect to SEQ ID NOS:5-6.

In various embodiments, the first RSV F sequence is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to positions 26-494 of the second RSV F sequence or positions 26-516 of the third RSV F sequence.

The first RSV F sequence of the recombinant RSV F proteins preferably comprise one or more modifications with respect to the second or third RSV F sequences. These modifications include any one or more of: a residue other than serine at a position corresponding to position 55 of the second or third RSV F sequence; a residue other than glutamate at a position corresponding to position 60 of the second or third RSV F sequence; a residue other than asparagine at a position corresponding to position 70 of the second or third RSV F sequence; a residue other than glutamine at a position corresponding to position 94 of the second or third RSV F sequence; a residue other than methionine at a position corresponding to position 97 of the second or third RSV F sequence; a residue other than serine at a position corresponding to position 128 of the second RSV F sequence or position 150 of the third RSV F sequence; a residue other than glycine at a position corresponding to position 129 of the second RSV F sequence or position 151 of the third RSV F sequence; a residue other than asparagine at a position corresponding to position 153 of the second RSV F sequence or position 175 of the third RSV F sequence; a residue other than glutamine at a position corresponding to position 203 of the second RSV F sequence or position 225 of the third RSV F sequence; a residue other than asparagine at a position corresponding to position 205 of the second RSV F sequence or position 227 of the third RSV F sequence; a residue other than valine at a position corresponding to position 256 of the second RSV F sequence or position 278 of the third RSV F sequence; a residue other than serine at a position corresponding to position 308 of the second RSV F sequence or position 330 of the third RSV F sequence; a residue other than threonine at a position corresponding to position 315 of the second RSV F sequence or position 337 of the third RSV F sequence; a residue other than glutamate at a position corresponding to position 356 of the second RSV F sequence or position 378 of the third RSV F sequence; a residue other than asparagine at a position corresponding to position 358 of the second RSV F sequence or position 380 of the third RSV F sequence; a residue other than glutamate at a position corresponding to position 465 of the second RSV F sequence or position 487 of the third RSV F sequence; cysteines at positions corresponding to positions 52 and 128 of the second RSV F sequence or positions 52 and 150 of the third RSV F sequence; cysteines at positions corresponding to positions 55 and 166 of the second RSV F sequence or positions 55 and 188 of the third RSV F sequence; cysteines at positions corresponding to positions 60 and 174 of the second RSV F sequence or positions 60 and 196 of the third RSV F sequence; cysteines at positions corresponding to positions 135 and 161 of the second RSV F sequence or positions 157 and 183 of the third RSV F sequence; and cysteines at positions corresponding to positions 421 and 444 of the second RSV F sequence or positions 443 and 466 of the third RSV F sequence. In some embodiments, any one or more of these modifications confers enhanced stability of a prefusion state of the recombinant RSV F protein over a postfusion state. See the following examples for a description of the prefusion and postfusion states as well as methods for detecting same.

The first RSV F sequence can include any number of the above-referenced modifications. In various embodiments, the first RSV F sequence comprises 1 or more, 2 or more, 3 or more, 4 or more, 5 or more, 6 or more, 7 or more, 8 or more, 9 or more, 10 or more, or 11 or more of the above-referenced modifications.

In some embodiments, the first RSV F sequence comprises one or more, two or more, three or more, four or more, five or more six or more, or each of: a residue other than serine at a position corresponding to position 55 of the second or third RSV F sequence; a residue other than glutamate at a position corresponding to position 60 of the second or third RSV F sequence; a residue other than serine at a position corresponding to position 128 of the second RSV F sequence or position 150 of the third RSV F sequence; a residue other than asparagine at a position corresponding to position 153 of the second RSV F sequence or position 175 of the third RSV F sequence; a residue other than asparagine at a position corresponding to position 205 of the second RSV F sequence or position 227 of the third RSV F sequence; a residue other than asparagine at a position corresponding to position 358 of the second RSV F sequence or position 380 of the third RSV F sequence; and a residue other than glutamate at a position corresponding to position 465 of the second RSV F sequence or position 487 of the third RSV F sequence. Exemplary recombinant RSV F proteins comprising these modifications include R-1b (SEQ ID NO:2), R-02 (SEQ ID NO:3), and R-03 (SEQ ID NO:4).

In some embodiments, the residue other than the serine at the position corresponding to position 55 of the second or third RSV F sequence is alanine, isoleucine, leucine, valine, cysteine, or a conservative variant of alanine, isoleucine, leucine, or valine. In some embodiments, the residue other than the glutamate at the position corresponding to position 60 of the second or third RSV F sequence is isoleucine, phenylalanine, cysteine, or a conservative variant of isoleucine or phenylalanine. In some embodiments, the residue other than the asparagine at the position corresponding to position 70 of the second or third RSV F sequence is glutamine or a conservative variant of glutamine. In some embodiments, the residue other than the glutamine at the position corresponding to position 94 of the second or third RSV F sequence is arginine or a conservative variant of arginine. In some embodiments, the residue other than the methionine at the position corresponding to position 97 of the second or third RSV F sequence is isoleucine, threonine, or a conservative variant of isoleucine or threonine. In some embodiments, the residue other than the serine at the position corresponding to position 128 of the second RSV F sequence or position 150 of the third RSV F sequence is glutamate, leucine, cysteine, or a conservative variant of glutamate or leucine. In some embodiments, the residue other than the glycine at the position corresponding to position 129 of the second RSV F sequence or position 151 of the third RSV F sequence is alanine or a conservative variant of alanine. In some embodiments, the residue other than the asparagine at the position corresponding to position 153 of the second RSV F sequence or position 175 of the third RSV F sequence is arginine or a conservative variant of arginine. In some embodiments, the residue other than the glutamine at the position corresponding to position 203 of the second RSV F sequence or position 225 of the third RSV F sequence is glutamate or a conservative variant of glutamate. In some embodiments, the residue other than the asparagine at the position corresponding to position 205 of the second RSV F sequence or position 227 of the third RSV F sequence is leucine, phenylalanine, or a conservative variant of leucine or phenylalanine. In some embodiments, the residue other than the valine at the position corresponding to position 256 of the second RSV F sequence or position 278 of the third RSV F sequence is leucine or a conservative variant of leucine. In some embodiments, the residue other than the serine at the position corresponding to position 308 of the second RSV F sequence or position 330 of the third RSV F sequence is aspartate or a conservative variant of aspartate. In some embodiments, the residue other than the threonine at the position corresponding to position 315 of the second RSV F sequence or position 337 of the third RSV F sequence is leucine or a conservative variant of leucine. In some embodiments, the residue other than the glutamate at the position corresponding to position 356 of the second RSV F sequence or position 378 of the third RSV F sequence is lysine or a conservative variant of lysine. In some embodiments, the residue other than the asparagine at the position corresponding to position 358 of the second RSV F sequence or position 380 of the third RSV F sequence is lysine or a conservative variant of lysine. In some embodiments, the residue other than the glutamate at the position corresponding to position 465 of the second RSV F sequence or position 487 of the third RSV F sequence is asparagine, valine or a conservative variant of asparagine or valine.

In some embodiments, the residue other than the serine at the position corresponding to position 55 of the second or third RSV F sequence is alanine, isoleucine, leucine, valine, or cysteine. In some embodiments, the residue other than the glutamate at the position corresponding to position 60 of the second or third RSV F sequence is isoleucine, phenylalanine, or cysteine. In some embodiments, the residue other than the asparagine at the position corresponding to position 70 of the second or third RSV F sequence is glutamine. In some embodiments, the residue other than the glutamine at the position corresponding to position 94 of the second or third RSV F sequence is arginine. In some embodiments, the residue other than the methionine at the position corresponding to position 97 of the second or third RSV F sequence is isoleucine or threonine. In some embodiments, the residue other than the serine at the position corresponding to position 128 of the second RSV F sequence or position 150 of the third RSV F sequence is glutamate, leucine, or cysteine. In some embodiments, the residue other than the glycine at the position corresponding to position 129 of the second RSV F sequence or position 151 of the third RSV F sequence is alanine. In some embodiments, the residue other than the asparagine at the position corresponding to position 153 of the second RSV F sequence or position 175 of the third RSV F sequence is arginine. In some embodiments, the residue other than the glutamine at the position corresponding to position 203 of the second RSV F sequence or position 225 of the third RSV F sequence is glutamate. In some embodiments, the residue other than the asparagine at the position corresponding to position 205 of the second RSV F sequence or position 227 of the third RSV F sequence is leucine or phenylalanine. In some embodiments, the residue other than the valine at the position corresponding to position 256 of the second RSV F sequence or position 278 of the third RSV F sequence is leucine. In some embodiments, the residue other than the serine at the position corresponding to position 308 of the second RSV F sequence or position 330 of the third RSV F sequence is aspartate. In some embodiments, the residue other than the threonine at the position corresponding to position 315 of the second RSV F sequence or position 337 of the third RSV F sequence is leucine. In some embodiments, the residue other than the glutamate at the position corresponding to position 356 of the second RSV F sequence or position 378 of the third RSV F sequence is lysine. In some embodiments, the residue other than the asparagine at the position corresponding to position 358 of the second RSV F sequence or position 380 of the third RSV F sequence is lysine. In some embodiments, the residue other than the glutamate at the position corresponding to position 465 of the second RSV F sequence or position 487 of the third RSV F sequence is asparagine or valine.

In some embodiments, the first RSV F sequence comprises: phenylalanine or cysteine at the position corresponding to position 60 of the second or third RSV F sequence; arginine at the position corresponding to position 153 of the second RSV F sequence or position 175 of the third RSV F sequence; and lysine at the position corresponding to position 358 of the second RSV F sequence or position 380 of the third RSV F sequence. Exemplary recombinant RSV F proteins comprising these modifications include R-1b (SEQ ID NO:2), R-02 (SEQ ID NO:3), and R-03 (SEQ ID NO:4). In some embodiments, the first RSV F sequence comprises: alanine or cysteine at the position corresponding to position 55 of the second or third RSV F sequence; glutamate or cysteine at the position corresponding to position 128 of the second RSV F sequence or position 150 of the third RSV F sequence; leucine at the position corresponding to position 205 of the second RSV F sequence or position 227 of the third RSV F sequence; and asparagine at the position corresponding to position 465 of the second RSV F sequence or position 487 of the third RSV F sequence. Exemplary recombinant RSV F proteins comprising these modifications include R-1b (SEQ ID NO:2).

In some embodiments, the first RSV F sequence of the recombinant RSV F protein comprises cysteines at various positions. The cysteines can confer enhanced stability of a prefusion state of the recombinant RSV F protein over a postfusion state, in some versions, by forming disulfide bonds. In some embodiments, the first RSV F sequence comprises cysteines at positions corresponding to positions 52 and 128 of the second RSV F sequence or positions 52 and 150 of the third RSV F sequence. In some embodiments, the first RSV F sequence comprises cysteines at positions corresponding to positions 55 and 166 of the second RSV F sequence or positions 55 and 188 of the third RSV F sequence. In some embodiments, the first RSV F sequence comprises cysteines at positions corresponding to positions 60 and 174 of the second RSV F sequence or positions 60 and 196 of the third RSV F sequence. In some embodiments, the first RSV F sequence comprises cysteines at positions corresponding to positions 135 and 161 of the second RSV F sequence or positions 157 and 183 of the third RSV F sequence. In some embodiments, the first RSV F sequence comprises cysteines at positions corresponding to positions 421 and 444 of the second RSV F sequence or positions 443 and 466 of the third RSV F sequence.

The recombinant hMPV proteins of the invention preferably comprise a first hMPV F sequence at least 80% identical to positions 19-489 of a second hMPV F sequence selected from the group consisting of SEQ ID NOS:7-15. SEQ ID NOS:7-15 are exemplary hMPV F constructs described herein (see Table 1 and the following examples). Positions 19-489 of SEQ ID NOS:7-15 include portions corresponding to residues 1-95 and 103-472 of the hMPV F protein (excluding the signal sequence, which is cleaved after expression), a modified cleavage site “ENPRRRR” (positions 96-102 of SEQ ID NOS:7-15), and an A185P mutation (see Table 2 and the following examples).

In various embodiments, the first hMPV F sequence is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to positions 19-489 of the second hMPV F sequence.

The first hMPV F sequence of the recombinant hMPV F proteins preferably comprise one or more modifications with respect to the second hMPV F sequence. These modifications include any one or more of: a residue other than glycine at a position corresponding to position 42 of the second hMPV F sequence; a residue other than alanine at a position corresponding to position 90 of the second hMPV F sequence; a residue other than glycine at a position corresponding to position 106 of the second hMPV F sequence; a residue other than alanine at a position corresponding to position 107 of the second hMPV F sequence; a residue other than threonine at a position corresponding to position 114 of the second hMPV F sequence; a residue other than alanine at a position corresponding to position 116 of the second hMPV F sequence; a residue other than leucine at a position corresponding to position 130 of the second hMPV F sequence; a residue other than lysine at a position corresponding to position 143 of the second hMPV F sequence; a residue other than serine at a position corresponding to position 149 of the second hMPV F sequence; a residue other than threonine at a position corresponding to position 150 of the second hMPV F sequence; a residue other than glycine at a position corresponding to position 152 of the second hMPV F sequence; a residue other than alanine at a position corresponding to position 159 of the second hMPV F sequence; a residue other than threonine at a position corresponding to position 160 of the second hMPV F sequence; a residue other than valine at a position corresponding to position 162 of the second hMPV F sequence; a residue other than leucine at a position corresponding to position 187 of the second hMPV F sequence; a residue other than valine at a position corresponding to position 191 of the second hMPV F sequence; a residue other than valine at a position corresponding to position 203 of the second hMPV F sequence; a residue other than aspartate at a position corresponding to position 209 of the second hMPV F sequence; a residue other than alanine at a position corresponding to position 216 of the second hMPV F sequence; a residue other than serine at a position corresponding to position 237 of the second hMPV F sequence; a residue other than glycine at a position corresponding to position 277 of the second hMPV F sequence; a residue other than alanine at a position corresponding to position 314 of the second hMPV F sequence; a residue other than serine at a position corresponding to position 371 of the second hMPV F sequence; a residue other than glycine at a position corresponding to position 393 of the second hMPV F sequence; a residue other than valine at a position corresponding to position 430 of the second hMPV F sequence; a residue other than isoleucine at a position corresponding to position 437 of the second hMPV F sequence; a residue other than valine at a position corresponding to position 449 of the second hMPV F sequence; a residue other than glutamate at a position corresponding to position 453 of the second hMPV F sequence; cysteines at positions corresponding to positions 43 and 120 of the second hMPV F sequence; cysteines at positions corresponding to positions 45 and 157 of the second hMPV F sequence; cysteines at positions corresponding to positions 113 and 336 of the second hMPV F sequence; cysteines at positions corresponding to positions 115 and 375 of the second hMPV F sequence; cysteines at positions corresponding to positions 123 and 429 of the second hMPV F sequence; and cysteines at positions corresponding to positions 211 and 253 of the second hMPV F sequence. In some embodiments, any one or more of these modifications confers enhanced stability of a prefusion state of the recombinant hMPV F protein over a postfusion state. See the following examples for a description of the prefusion and postfusion states as well as methods for detecting same.

The first hMPV F sequence can include any number of the above-referenced modifications. In various embodiments, the first hMPV F sequence comprises 1 or more, 2 or more, 3 or more, 4 or more, 5 or more, 6 or more, 7 or more, 8 or more, 9 or more, 10 or more of, 11 or more, 12 or more, 13 or more, 14 or more, 15 or more, 16 or more, 17 or more, or 18 or more of the above-referenced modifications.

In some embodiments, the first hMPV F sequence comprises 1 or more, 2 or more, 3 or more, 4 or more, 5 or more, 6 or more, 7 or more, 8 or more, 9 or more, 10 or more, or 11 or more of: a residue other than glycine at a position corresponding to position 42 of the second hMPV F sequence; a residue other than alanine at a position corresponding to position 90 of the second hMPV F sequence; a residue other than glycine at a position corresponding to position 106 of the second hMPV F sequence; a residue other than alanine at a position corresponding to position 107 of the second hMPV F sequence; a residue other than threonine at a position corresponding to position 114 of the second hMPV F sequence; a residue other than alanine at a position corresponding to position 116 of the second hMPV F sequence; a residue other than leucine at a position corresponding to position 130 of the second hMPV F sequence; a residue other than alanine at a position corresponding to position 159 of the second hMPV F sequence; a residue other than valine at a position corresponding to position 203 of the second hMPV F sequence; a residue other than aspartate at a position corresponding to position 209 of the second hMPV F sequence; a residue other than serine at a position corresponding to position 237 of the second hMPV F sequence; a residue other than glycine at a position corresponding to position 277 of the second hMPV F sequence; a residue other than alanine at a position corresponding to position 314 of the second hMPV F sequence; a residue other than serine at a position corresponding to position 371 of the second hMPV F sequence; a residue other than glycine at a position corresponding to position 393 of the second hMPV F sequence; a residue other than valine at a position corresponding to position 430 of the second hMPV F sequence; a residue other than valine at a position corresponding to position 449 of the second hMPV F sequence; and a residue other than glutamate at a position corresponding to position 453 of the second hMPV F sequence. Exemplary recombinant hMPV F proteins comprising these modifications include M-102 (SEQ ID NO:8), M-104 (SEQ ID NO:9), M-302 (SEQ ID NO: 10), M-305 (SEQ ID NO:11), M-374 (SEQ ID NO:12), and M-404 (SEQ ID NO:13).

In some embodiments, the first hMPV F sequence comprises 1 or more, 2 or more, 3 or more, 4 or more, 5 or more, 6 or more, 7 or more, 8 or more, 9 or more, 10 or more, or 11 or more of: a residue other than alanine at a position corresponding to position 90 of the second hMPV F sequence; a residue other than glycine at a position corresponding to position 106 of the second hMPV F sequence; a residue other than alanine at a position corresponding to position 107 of the second hMPV F sequence; a residue other than alanine at a position corresponding to position 116 of the second hMPV F sequence; a residue other than leucine at a position corresponding to position 130 of the second hMPV F sequence; a residue other than alanine at a position corresponding to position 159 of the second hMPV F sequence; a residue other than aspartate at a position corresponding to position 209 of the second hMPV F sequence; a residue other than glycine at a position corresponding to position 277 of the second hMPV F sequence; a residue other than alanine at a position corresponding to position 314 of the second hMPV F sequence; a residue other than valine at a position corresponding to position 449 of the second hMPV F sequence; and a residue other than glutamate at a position corresponding to position 453 of the second hMPV F sequence. Exemplary recombinant hMPV F proteins comprising these modifications include M-102 (SEQ ID NO:8), M-104 (SEQ ID NO:9), M-302 (SEQ ID NO:10), M-305 (SEQ ID NO:11), M-374 (SEQ ID NO:12), and M-404 (SEQ ID NO:13), each of which comprises more than one of these mutations.

In some embodiments, the first hMPV F sequence comprises two or more, three or more, four or more, five or more, six or more, seven or more, or eight or more of: a residue other than alanine at a position corresponding to position 90 of the second hMPV F sequence; a residue other than glycine at a position corresponding to position 106 of the second hMPV F sequence; a residue other than threonine at a position corresponding to position 114 of the second hMPV F sequence; a residue other than leucine at a position corresponding to position 130 of the second hMPV F sequence; a residue other than alanine at a position corresponding to position 159 of the second hMPV F sequence; a residue other than valine at a position corresponding to position 203 of the second hMPV F sequence; a residue other than glycine at a position corresponding to position 277 of the second hMPV F sequence; a residue other than alanine at a position corresponding to position 314 of the second hMPV F sequence; a residue other than valine at a position corresponding to position 430 of the second hMPV F sequence; a residue other than valine at a position corresponding to position 449 of the second hMPV F sequence; and a residue other than glutamate at a position corresponding to position 453 of the second hMPV F sequence. Exemplary recombinant hMPV F proteins comprising these modifications include M-104 (SEQ ID NO:9) and M-305 (SEQ ID NO:11).

In some embodiments, the first hMPV F sequence comprises two or more, three or more, four or more, five or more, six or more, or each of: a residue other than alanine at a position corresponding to position 90 of the second hMPV F sequence; a residue other than threonine at a position corresponding to position 114 of the second hMPV F sequence; a residue other than leucine at a position corresponding to position 130 of the second hMPV F sequence; a residue other than alanine at a position corresponding to position 159 of the second hMPV F sequence; a residue other than valine at a position corresponding to position 203 of the second hMPV F sequence; a residue other than valine at a position corresponding to position 430 of the second hMPV F sequence; and a residue other than valine at a position corresponding to position 449 of the second hMPV F sequence. Exemplary recombinant hMPV F proteins comprising these modifications include M-104 (SEQ ID NO:9).

In some embodiments, the first hMPV F sequence comprises two or more, three or more, four or more, five or more, six or more, seven or more, or each of: a residue other than alanine at a position corresponding to position 90 of the second hMPV F sequence; a residue other than glycine at a position corresponding to position 106 of the second hMPV F sequence; a residue other than leucine at a position corresponding to position 130 of the second hMPV F sequence; a residue other than alanine at a position corresponding to position 159 of the second hMPV F sequence; a residue other than glycine at a position corresponding to position 277 of the second hMPV F sequence; a residue other than alanine at a position corresponding to position 314 of the second hMPV F sequence; a residue other than valine at a position corresponding to position 449 of the second hMPV F sequence; and a residue other than glutamate at a position corresponding to position 453 of the second hMPV F sequence. Exemplary recombinant hMPV F proteins comprising these modifications include M-305 (SEQ ID NO:11).

In some embodiments, the first hMPV F sequence comprises one or more, two or more, three or more, or each of: a residue other than alanine at a position corresponding to position 90 of the second hMPV F sequence; a residue other than leucine at a position corresponding to position 130 of the second hMPV F sequence; a residue other than alanine at a position corresponding to position 159 of the second hMPV F sequence; and a residue other than valine at a position corresponding to position 449 of the second hMPV F sequence. Exemplary recombinant hMPV F proteins comprising these modifications include M-104 (SEQ ID NO:9) and M-305 (SEQ ID NO:11).

In some embodiments, the residue other than the glycine at the position corresponding to position 42 of the second hMPV F sequence is aspartate or a conservative variant of aspartate. In some embodiments, the residue other than the alanine at the position corresponding to position 90 of the second hMPV F sequence is asparagine or a conservative variant of asparagine. In some embodiments, the residue other than the glycine at the position corresponding to position 106 of the second hMPV F sequence is arginine, tryptophan, phenylalanine or a conservative variant of arginine, tryptophan, or phenylalanine. In some embodiments, the residue other than the alanine at the position corresponding to position 107 of the second hMPV F sequence is leucine, phenylalanine, or a conservative variant of leucine or phenylalanine. In some embodiments, the residue other than the threonine at the position corresponding to position 114 of the second hMPV F sequence is glutamate or a conservative variant of glutamate. In some embodiments, the residue other than the alanine at the position corresponding to position 116 of the second hMPV F sequence is valine or a conservative variant of valine. In some embodiments, the residue other than the leucine at the position corresponding to position 130 of the second hMPV F sequence is aspartate or a conservative variant of aspartate. In some embodiments, the residue other than the lysine at the position corresponding to position 143 of the second hMPV F sequence is aspartate or a conservative variant of aspartate. In some embodiments, the residue other than the serine at the position corresponding to position 149 of the second hMPV F sequence is isoleucine, threonine, or a conservative variant of isoleucine or threonine. In some embodiments, the residue other than the threonine at the position corresponding to position 150 of the second hMPV F sequence is aspartate or a conservative variant of aspartate. In some embodiments, the residue other than the glycine at the position corresponding to position 152 of the second hMPV F sequence is lysine or a conservative variant of lysine. In some embodiments, the residue other than the alanine at the position corresponding to position 159 of the second hMPV F sequence is leucine, isoleucine or a conservative variant of leucine or isoleucine. In some embodiments, the residue other than the threonine at the position corresponding to position 160 of the second hMPV F sequence is phenylalanine or a conservative variant of phenylalanine. In some embodiments, the residue other than the valine at the position corresponding to position 162 of the second hMPV F sequence is isoleucine or a conservative variant of isoleucine. In some embodiments, the residue other than the leucine at the position corresponding to position 187 of the second hMPV F sequence is isoleucine or a conservative variant of isoleucine. In some embodiments, the residue other than the valine at the position corresponding to position 191 of the second hMPV F sequence is isoleucine or a conservative variant of isoleucine. In some embodiments, the residue other than the valine at the position corresponding to position 203 of the second hMPV F sequence is isoleucine or a conservative variant of isoleucine. In some embodiments, the residue other than the aspartate at the position corresponding to position 209 of the second hMPV F sequence is glutamate or a conservative variant of glutamate. In some embodiments, the residue other than the alanine at the position corresponding to position 216 of the second hMPV F sequence is arginine, serine, or a conservative variant of arginine or serine. In some embodiments, the residue other than the serine at the position corresponding to position 237 of the second hMPV F sequence is histidine or a conservative variant of histidine. In some embodiments, the residue other than the glycine at the position corresponding to position 277 of the second hMPV F sequence is aspartate, glutamate, lysine or a conservative variant of aspartate, glutamate, or lysine. In some embodiments, the residue other than the alanine at the position corresponding to position 314 of the second hMPV F sequence is asparagine, lysine, or a conservative variant of asparagine or lysine. In some embodiments, the residue other than the serine at the position corresponding to position 371 of the second hMPV F sequence is proline or a conservative variant of proline. In some embodiments, the residue other than the glycine at the position corresponding to position 393 of the second hMPV F sequence is serine or a conservative variant of serine. In some embodiments, the residue other than the valine at the position corresponding to position 430 of the second hMPV F sequence is glutamine or a conservative variant of glutamine. In some embodiments, the residue other than the isoleucine at the position corresponding to position 437 of the second hMPV F sequence is arginine or a conservative variant of arginine. In some embodiments, the residue other than the valine at the position corresponding to position 449 of the second hMPV F sequence is aspartate, glutamate, or a conservative variant of aspartate or glutamate. In some embodiments, the residue other than the glutamate at the position corresponding to position 453 of the second hMPV F sequence is proline or a conservative variant of proline.

In some embodiments, the residue other than the glycine at the position corresponding to position 42 of the second hMPV F sequence is aspartate. In some embodiments, the residue other than the alanine at the position corresponding to position 90 of the second hMPV F sequence is asparagine. In some embodiments, the residue other than the glycine at the position corresponding to position 106 of the second hMPV F sequence is arginine, tryptophan, or phenylalanine. In some embodiments, the residue other than the alanine at the position corresponding to position 107 of the second hMPV F sequence is leucine or phenylalanine. In some embodiments, the residue other than the threonine at the position corresponding to position 114 of the second hMPV F sequence is glutamate. In some embodiments, the residue other than the alanine at the position corresponding to position 116 of the second hMPV F sequence is valine. In some embodiments, the residue other than the leucine at the position corresponding to position 130 of the second hMPV F sequence is aspartate. In some embodiments, the residue other than the lysine at the position corresponding to position 143 of the second hMPV F sequence is aspartate. In some embodiments, the residue other than the serine at the position corresponding to position 149 of the second hMPV F sequence is isoleucine or threonine. In some embodiments, the residue other than the threonine at the position corresponding to position 150 of the second hMPV F sequence is aspartate. In some embodiments, the residue other than the glycine at the position corresponding to position 152 of the second hMPV F sequence is lysine. In some embodiments, the residue other than the alanine at the position corresponding to position 159 of the second hMPV F sequence is leucine or isoleucine. In some embodiments, the residue other than the threonine at the position corresponding to position 160 of the second hMPV F sequence is phenylalanine. In some embodiments, the residue other than the valine at the position corresponding to position 162 of the second hMPV F sequence is isoleucine. In some embodiments, the residue other than the leucine at the position corresponding to position 187 of the second hMPV F sequence is isoleucine. In some embodiments, the residue other than the valine at the position corresponding to position 191 of the second hMPV F sequence is isoleucine. In some embodiments, the residue other than the valine at the position corresponding to position 203 of the second hMPV F sequence is isoleucine. In some embodiments, the residue other than the aspartate at the position corresponding to position 209 of the second hMPV F sequence is glutamate. In some embodiments, the residue other than the alanine at the position corresponding to position 216 of the second hMPV F sequence is arginine or serine. In some embodiments, the residue other than the serine at the position corresponding to position 237 of the second hMPV F sequence is histidine. In some embodiments, the residue other than the glycine at the position corresponding to position 277 of the second hMPV F sequence is aspartate, glutamate, or lysine. In some embodiments, the residue other than the alanine at the position corresponding to position 314 of the second hMPV F sequence is asparagine or lysine. In some embodiments, the residue other than the serine at the position corresponding to position 371 of the second hMPV F sequence is proline. In some embodiments, the residue other than the glycine at the position corresponding to position 393 of the second hMPV F sequence is serine. In some embodiments, the residue other than the valine at the position corresponding to position 430 of the second hMPV F sequence is glutamine. In some embodiments, the residue other than the isoleucine at the position corresponding to position 437 of the second hMPV F sequence is arginine. In some embodiments, the residue other than the valine at the position corresponding to position 449 of the second hMPV F sequence is aspartate or glutamate. In some embodiments, the residue other than the glutamate at the position corresponding to position 453 of the second hMPV F sequence is proline.

In some embodiments, the residue other than the alanine at the position corresponding to position 90 of the second hMPV F sequence is asparagine. In some embodiments, the residue other than the glycine at the position corresponding to position 106 of the second hMPV F sequence is arginine. In some embodiments, the residue other than the threonine at the position corresponding to position 114 of the second hMPV F sequence is glutamate. In some embodiments, the residue other than the leucine at the position corresponding to position 130 of the second hMPV F sequence is aspartate. In some embodiments, the residue other than the alanine at the position corresponding to position 159 of the second hMPV F sequence is leucine or isoleucine. In some embodiments, the residue other than the valine at the position corresponding to position 203 of the second hMPV F sequence is isoleucine. In some embodiments, the residue other than the glycine at the position corresponding to position 277 of the second hMPV F sequence is aspartate. In some embodiments, the residue other than the alanine at the position corresponding to position 314 of the second hMPV F sequence is lysine. In some embodiments, the residue other than the valine at the position corresponding to position 430 of the second hMPV F sequence is glutamine. In some embodiments, the residue other than the valine at the position corresponding to position 449 of the second hMPV F sequence is aspartate. In some embodiments, the residue other than the glutamate at the position corresponding to position 453 of the second hMPV F sequence is proline. Exemplary recombinant hMPV F proteins comprising these modifications include M-104 (SEQ ID NO:9) and M-305 (SEQ ID NO:11).

In some embodiments, the first hMPV F sequence of the recombinant hMPV F protein comprises cysteines at various positions. The cysteines can confer enhanced stability of a prefusion state of the recombinant hMPV F protein over a postfusion state, in some versions, by forming disulfide bonds. In some embodiments, the first hMPV F sequence comprises cysteines at positions corresponding to positions 43 and 120 of the second hMPV F sequence. In some embodiments, the first hMPV F sequence comprises cysteines at positions corresponding to positions 45 and 157 of the second hMPV F sequence. In some embodiments, the first hMPV F sequence comprises cysteines at positions corresponding to positions 113 and 336 of the second hMPV F sequence. In some embodiments, the first hMPV F sequence comprises cysteines at positions corresponding to positions 115 and 375 of the second hMPV F sequence. In some embodiments, the first hMPV F sequence comprises cysteines at positions corresponding to positions 123 and 429 of the second hMPV F sequence. In some embodiments, the first hMPV F sequence comprises cysteines at positions corresponding to positions 211 and 253 of the second hMPV F sequence.

The recombinant SARS-COV-2 S proteins preferably comprise a first SARS-COV-2 S sequence at least 80% identical to positions 15-1208 of a second SARS-COV-2 S sequence selected from the group consisting of SEQ ID NOS: 16-20. SEQ ID NOS: 16-20 are exemplary SARS-COV-2 S constructs described herein (see Table 1 and the following examples). Positions 15-1208 of SEQ ID NOS: 16-20 include portions corresponding to portions of the SARS-COV-2 S-2P protein, with two proline substitutions at residues 986 and 987, and a “GSAS” (positions 682-685 of SEQ ID NOS: 16-20) linker replacing the furin cleavage site (see Table 2 and the following examples).

In various embodiments, the first SARS-COV-2 S sequence is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to positions 15-1208 of the second SARS-COV-2 S sequence.

The first SARS-COV-2 S sequence of the recombinant SARS-COV-2 S proteins preferably comprise one or more modifications with respect to the second SARS-COV-2 S sequence. These modifications include any one or more of: a residue other than alanine at a position corresponding to position 706 of the second SARS-COV-2 S sequence; a residue other than threonine at a position corresponding to position 732 of the second SARS-COV-2 S sequence; a residue other than glycine at a position corresponding to position 744 of the second SARS-COV-2 S sequence; a residue other than alanine at a position corresponding to position 766 of the second SARS-COV-2 S sequence; a residue other than glycine at a position corresponding to position 769 of the second SARS-COV-2 S sequence; a residue other than threonine at a position corresponding to position 827 of the second SARS-COV-2 S sequence; a residue other than isoleucine at a position corresponding to position 844 of the second SARS-CoV-2 S sequence; a residue other than arginine at a position corresponding to position 847 of the second SARS-COV-2 S sequence; a residue other than asparagine at a position corresponding to position 856 of the second SARS-COV-2 S sequence; a residue other than alanine at a position corresponding to position 899 of the second SARS-COV-2 S sequence; a residue other than glycine at a position corresponding to position 908 of the second SARS-CoV-2 S sequence; a residue other than threonine at a position corresponding to position 912 of the second SARS-COV-2 S sequence; a residue other than leucine at a position corresponding to position 916 of the second SARS-COV-2 S sequence; a residue other than tyrosine at a position corresponding to position 917 of the second SARS-COV-2 S sequence; a residue other than threonine at a position corresponding to position 941 of the second SARS-CoV-2 S sequence; a residue other than alanine at a position corresponding to position 942 of the second SARS-COV-2 S sequence; a residue other than asparagine at a position corresponding to position 955 of the second SARS-COV-2 S sequence; a residue other than alanine at a position corresponding to position 956 of the second SARS-COV-2 S sequence; a residue other than lysine at a position corresponding to position 964 of the second SARS-COV-2 S sequence; a residue other than aspartate at a position corresponding to position 985 of the second SARS-COV-2 S sequence; a residue other than glutamate at a position corresponding to position 990 of the second SARS-COV-2 S sequence; a residue other than threonine at a position corresponding to position 998 of the second SARS-COV-2 S sequence; a residue other than glutamine at a position corresponding to position 1005 of the second SARS-COV-2 S sequence; a residue other than threonine at a position corresponding to position 1009 of the second SARS-COV-2 S sequence; a residue other than leucine at a position corresponding to position 1012 of the second SARS-COV-2 S sequence; a residue other than isoleucine at a position corresponding to position 1013 of the second SARS-COV-2 S sequence; a residue other than alanine at a position corresponding to position 1016 of the second SARS-COV-2 S sequence; a residue other than alanine at a position corresponding to position 1020 of the second SARS-COV-2 S sequence; a residue other than asparagine at a position corresponding to position 1023 of the second SARS-COV-2 S sequence; a residue other than serine at a position corresponding to position 1051 of the second SARS-COV-2 S sequence; and a residue other than proline at a position corresponding to position 1143 of the second SARS-COV-2 S sequence. In some embodiments, any one or more of these modifications confers enhanced stability of a prefusion state of the recombinant SARS-COV-2 S protein over a postfusion state. See the following examples for a description of the prefusion and postfusion states as well as methods for detecting same.

The first SARS-COV-2 S sequence can include any number of the above-referenced modifications. In various embodiments, the first SARS-COV-2 S sequence comprises 1 or more, 2 or more, 3 or more, 4 or more, 5 or more, 6 or more, 7 or more, 8 or more, 9 or more, 10 or more, 11 or more, or 12 or more of the above-referenced modifications.

In some embodiments, the first SARS-COV-2 S sequence comprises one or more, two or more, three or more, four or more, or five or more of: a residue other than glycine at a position corresponding to position 769 of the second SARS-COV-2 S sequence; a residue other than leucine at a position corresponding to position 916 of the second SARS-COV-2 S sequence; a residue other than threonine at a position corresponding to position 941 of the second SARS-COV-2 S sequence; a residue other than alanine at a position corresponding to position 956 of the second SARS-COV-2 S sequence; a residue other than aspartate at a position corresponding to position 985 of the second SARS-COV-2 S sequence; a residue other than glutamate at a position corresponding to position 990 of the second SARS-COV-2 S sequence; a residue other than alanine at a position corresponding to position 1016 of the second SARS-CoV-2 S sequence; and a residue other than proline at a position corresponding to position 1143 of the second SARS-COV-2 S sequence. Exemplary recombinant SARS-COV-2 S proteins comprising these modifications include Spk-M (SEQ ID NO:17), Spk-F (SEQ ID NO:18), Spk-R (SEQ ID NO:19), and Spk-A (SEQ ID NO:20).

In some embodiments, the first SARS-COV-2 S sequence comprises a residue other than threonine at a position corresponding to position 941 of the second SARS-COV-2 S sequence. Exemplary recombinant SARS-COV-2 S proteins comprising these modifications include Spk-M (SEQ ID NO:17), Spk-F (SEQ ID NO:18), Spk-R (SEQ ID NO:19), and Spk-A (SEQ ID NO:20).

In some embodiments, the first SARS-COV-2 S sequence comprises one or more, two or more, three or more, four or more, five or more, six or more, seven or more, eight or more, or each of: a residue other than asparagine at a position corresponding to position 856 of the second SARS-COV-2 S sequence; a residue other than alanine at a position corresponding to position 899 of the second SARS-COV-2 S sequence; a residue other than leucine at a position corresponding to position 916 of the second SARS-COV-2 S sequence; a residue other than tyrosine at a position corresponding to position 917 of the second SARS-COV-2 S sequence; a residue other than threonine at a position corresponding to position 941 of the second SARS-CoV-2 S sequence; a residue other than alanine at a position corresponding to position 956 of the second SARS-COV-2 S sequence; a residue other than lysine at a position corresponding to position 964 of the second SARS-COV-2 S sequence; a residue other than aspartate at a position corresponding to position 985 of the second SARS-COV-2 S sequence; and a residue other than proline at a position corresponding to position 1143 of the second SARS-COV-2 S sequence. Exemplary recombinant SARS-COV-2 S proteins comprising these modifications include Spk-M (SEQ ID NO:17).

In some embodiments, the first SARS-COV-2 S sequence comprises one or more, two or more, three or more, four or more, five or more, or each of: a residue other than glycine at a position corresponding to position 769 of the second SARS-COV-2 S sequence; a residue other than threonine at a position corresponding to position 941 of the second SARS-COV-2 S sequence; a residue other than alanine at a position corresponding to position 956 of the second SARS-COV-2 S sequence; a residue other than glutamate at a position corresponding to position 990 of the second SARS-COV-2 S sequence; a residue other than alanine at a position corresponding to position 1016 of the second SARS-COV-2 S sequence; and a residue other than proline at a position corresponding to position 1143 of the second SARS-COV-2 S sequence. Exemplary recombinant SARS-COV-2 S proteins comprising these modifications include Spk-F (SEQ ID NO:18).

In some embodiment, the first SARS-COV-2 S sequence comprises one or more, two or more, three or more, four or more, five or more, six or more, seven or more, or each of: a residue other than glycine at a position corresponding to position 744 of the second SARS-CoV-2 S sequence; a residue other than glycine at a position corresponding to position 769 of the second SARS-COV-2 S sequence; a residue other than threonine at a position corresponding to position 941 of the second SARS-COV-2 S sequence; a residue other than asparagine at a position corresponding to position 955 of the second SARS-COV-2 S sequence; a residue other than aspartate at a position corresponding to position 985 of the second SARS-COV-2 S sequence; a residue other than glutamate at a position corresponding to position 990 of the second SARS-COV-2 S sequence; a residue other than alanine at a position corresponding to position 1016 of the second SARS-COV-2 S sequence; and a residue other than proline at a position corresponding to position 1143 of the second SARS-COV-2 S sequence. Exemplary recombinant SARS-COV-2 S proteins comprising these modifications include Spk-R (SEQ ID NO:19).

In some embodiments, the first SARS-COV-2 S sequence comprises one or more, two or more, three or more, four or more, five or more, six or more, seven or more, eight or more, nine or more, ten or more, eleven or more, or each of: a residue other than glycine at a position corresponding to position 769 of the second SARS-COV-2 S sequence; a residue other than leucine at a position corresponding to position 916 of the second SARS-COV-2 S sequence; a residue other than threonine at a position corresponding to position 941 of the second SARS-CoV-2 S sequence; a residue other than alanine at a position corresponding to position 942 of the second SARS-COV-2 S sequence; a residue other than glutamine at a position corresponding to position 1005 of the second SARS-COV-2 S sequence; a residue other than threonine at a position corresponding to position 1009 of the second SARS-COV-2 S sequence; a residue other than leucine at a position corresponding to position 1012 of the second SARS-CoV-2 S sequence; a residue other than isoleucine at a position corresponding to position 1013 of the second SARS-COV-2 S sequence; a residue other than alanine at a position corresponding to position 1016 of the second SARS-COV-2 S sequence; a residue other than alanine at a position corresponding to position 1020 of the second SARS-COV-2 S sequence; a residue other than asparagine at a position corresponding to position 1023 of the second SARS-COV-2 S sequence; and a residue other than serine at a position corresponding to position 1051 of the second SARS-COV-2 S sequence. Exemplary recombinant SARS-COV-2 S proteins comprising these modifications Spk-A (SEQ ID NO:20).

In some embodiments, the residue other than alanine at the position corresponding to position 706 of the second SARS-COV-2 S sequence is proline or a conservative variant of proline. In some embodiments, the residue other than threonine at the position corresponding to position 732 of the second SARS-COV-2 S sequence is isoleucine or a conservative variant of isoleucine. In some embodiments, the residue other than glycine at the position corresponding to position 744 of the second SARS-COV-2 S sequence is threonine or a conservative variant of threonine. In some embodiments, the residue other than alanine at the position corresponding to position 766 of the second SARS-COV-2 S sequence is isoleucine, leucine, or a conservative variant of isoleucine or leucine. In some embodiments, the residue other than glycine at the position corresponding to position 769 of the second SARS-COV-2 S sequence is lysine or a conservative variant of lysine. In some embodiments, the residue other than threonine at the position corresponding to position 827 of the second SARS-COV-2 S sequence is asparagine, lysine, or a conservative variant of asparagine or lysine. In some embodiments, the residue other than isoleucine at the position corresponding to position 844 of the second SARS-COV-2 S sequence is glutamate, glutamine, or a conservative variant of glutamate or glutamine. In some embodiments, the residue other than arginine at the position corresponding to position 847 of the second SARS-COV-2 S sequence is alanine or a conservative variant of alanine. In some embodiments, the residue other than asparagine at the position corresponding to position 856 of the second SARS-COV-2 S sequence is leucine or a conservative variant of leucine. In some embodiments, the residue other than alanine at the position corresponding to position 899 of the second SARS-COV-2 S sequence is glutamine or a conservative variant of glutamine. In some embodiments, the residue other than glycine at the position corresponding to position 908 of the second SARS-COV-2 S sequence is alanine, asparagine, or a conservative variant of alanine or asparagine. In some embodiments, the residue other than threonine at the position corresponding to position 912 of the second SARS-CoV-2 S sequence is isoleucine, leucine, or a conservative variant of isoleucine or leucine. In some embodiments, the residue other than leucine at the position corresponding to position 916 of the second SARS-COV-2 S sequence is phenylalanine or a conservative variant of phenylalanine. In some embodiments, the residue other than tyrosine at the position corresponding to position 917 of the second SARS-COV-2 S sequence is tryptophan or a conservative variant of tryptophan. In some embodiments, the residue other than threonine at the position corresponding to position 941 of the second SARS-COV-2 S sequence is asparagine, aspartate, or a conservative variant of asparagine or aspartate. In some embodiments, the residue other than alanine at the position corresponding to position 942 of the second SARS-COV-2 S sequence is aspartate, proline, or a conservative variant of aspartate or proline. In some embodiments, the residue other than asparagine at the position corresponding to position 955 of the second SARS-COV-2 S sequence is aspartate or a conservative variant of aspartate. In some embodiments, the residue other than alanine at the position corresponding to position 956 of the second SARS-COV-2 S sequence is isoleucine, leucine, valine, or a conservative variant of isoleucine, leucine, or valine. In some embodiments, the residue other than lysine at the position corresponding to position 964 of the second SARS-COV-2 S sequence is glutamate or a conservative variant of glutamate. In some embodiments, the residue other than aspartate at the position corresponding to position 985 of the second SARS-COV-2 S sequence is asparagine or a conservative variant of asparagine. In some embodiments, the residue other than glutamate at the position corresponding to position 990 of the second SARS-COV-2 S sequence is arginine or a conservative variant of arginine. In some embodiments, the residue other than threonine at the position corresponding to position 998 of the second SARS-COV-2 S sequence is glutamine or a conservative variant of glutamine. In some embodiments, the residue other than glutamine at the position corresponding to position 1005 of the second SARS-COV-2 S sequence is methionine or a conservative variant of methionine. In some embodiments, the residue other than threonine at the position corresponding to position 1009 of the second SARS-COV-2 S sequence is isoleucine or a conservative variant of isoleucine. In some embodiments, the residue other than leucine at the position corresponding to position 1012 of the second SARS-COV-2 S sequence is glutamine or a conservative variant of glutamine. In some embodiments, the residue other than isoleucine at the position corresponding to position 1013 of the second SARS-COV-2 S sequence is leucine or a conservative variant of leucine. In some embodiments, the residue other than alanine at the position corresponding to position 1016 of the second SARS-COV-2 S sequence is isoleucine or a conservative variant of isoleucine. In some embodiments, the residue other than alanine at the position corresponding to position 1020 of the second SARS-CoV-2 S sequence is leucine or a conservative variant of leucine. In some embodiments, the residue other than asparagine at the position corresponding to position 1023 of the second SARS-COV-2 S sequence is lysine or a conservative variant of lysine. In some embodiments, the residue other than serine at the position corresponding to position 1051 of the second SARS-COV-2 S sequence is threonine or a conservative variant of threonine. In some embodiments, the residue other than proline at the position corresponding to position 1143 of the second SARS-COV-2 S sequence is glutamine, asparagine or a conservative variant of glutamine or asparagine.

In some embodiments, the residue other than alanine at the position corresponding to position 706 of the second SARS-COV-2 S sequence is proline. In some embodiments, the residue other than threonine at the position corresponding to position 732 of the second SARS-CoV-2 S sequence is isoleucine. In some embodiments, the residue other than glycine at the position corresponding to position 744 of the second SARS-COV-2 S sequence is threonine. In some embodiments, the residue other than alanine at the position corresponding to position 766 of the second SARS-COV-2 S sequence is isoleucine or leucine. In some embodiments, the residue other than glycine at the position corresponding to position 769 of the second SARS-COV-2 S sequence is lysine. In some embodiments, the residue other than threonine at the position corresponding to position 827 of the second SARS-COV-2 S sequence is asparagine or lysine. In some embodiments, the residue other than isoleucine at the position corresponding to position 844 of the second SARS-COV-2 S sequence is glutamate or glutamine. In some embodiments, the residue other than arginine at the position corresponding to position 847 of the second SARS-COV-2 S sequence is alanine. In some embodiments, the residue other than asparagine at the position corresponding to position 856 of the second SARS-COV-2 S sequence is leucine. In some embodiments, the residue other than alanine at the position corresponding to position 899 of the second SARS-COV-2 S sequence is glutamine. In some embodiments, the residue other than glycine at the position corresponding to position 908 of the second SARS-COV-2 S sequence is alanine or asparagine. In some embodiments, the residue other than threonine at the position corresponding to position 912 of the second SARS-COV-2 S sequence is isoleucine or leucine. In some embodiments, the residue other than leucine at the position corresponding to position 916 of the second SARS-CoV-2 S sequence is phenylalanine. In some embodiments, the residue other than tyrosine at the position corresponding to position 917 of the second SARS-COV-2 S sequence is tryptophan. In some embodiments, the residue other than threonine at the position corresponding to position 941 of the second SARS-COV-2 S sequence is asparagine or aspartate. In some embodiments, the residue other than alanine at the position corresponding to position 942 of the second SARS-COV-2 S sequence is aspartate or proline. In some embodiments, the residue other than asparagine at the position corresponding to position 955 of the second SARS-COV-2 S sequence is aspartate. In some embodiments, the residue other than alanine at the position corresponding to position 956 of the second SARS-COV-2 S sequence is isoleucine, leucine, or valine. In some embodiments, the residue other than lysine at the position corresponding to position 964 of the second SARS-COV-2 S sequence is glutamate. In some embodiments, the residue other than aspartate at the position corresponding to position 985 of the second SARS-COV-2 S sequence is asparagine. In some embodiments, the residue other than glutamate at the position corresponding to position 990 of the second SARS-COV-2 S sequence is arginine. In some embodiments, the residue other than threonine at the position corresponding to position 998 of the second SARS-COV-2 S sequence is glutamine. In some embodiments, the residue other than glutamine at the position corresponding to position 1005 of the second SARS-COV-2 S sequence is methionine. In some embodiments, the residue other than threonine at the position corresponding to position 1009 of the second SARS-COV-2 S sequence is isoleucine. In some embodiments, the residue other than leucine at the position corresponding to position 1012 of the second SARS-COV-2 S sequence is glutamine. In some embodiments, the residue other than isoleucine at the position corresponding to position 1013 of the second SARS-COV-2 S sequence is leucine. In some embodiments, the residue other than alanine at the position corresponding to position 1016 of the second SARS-COV-2 S sequence is isoleucine. In some embodiments, the residue other than alanine at the position corresponding to position 1020 of the second SARS-COV-2 S sequence is leucine. In some embodiments, the residue other than asparagine at the position corresponding to position 1023 of the second SARS-COV-2 S sequence is lysine. In some embodiments, the residue other than serine at the position corresponding to position 1051 of the second SARS-COV-2 S sequence is threonine. In some embodiments, the residue other than proline at the position corresponding to position 1143 of the second SARS-COV-2 S sequence is glutamine or asparagine.

In some embodiments, the residue other than glycine at the position corresponding to position 744 of the second SARS-COV-2 S sequence is threonine. In some embodiments, the residue other than glycine at the position corresponding to position 769 of the second SARS-CoV-2 S sequence is lysine. In some embodiments, the residue other than asparagine at the position corresponding to position 856 of the second SARS-COV-2 S sequence is leucine. In some embodiments, the residue other than alanine at the position corresponding to position 899 of the second SARS-COV-2 S sequence is glutamine. In some embodiments, the residue other than leucine at the position corresponding to position 916 of the second SARS-COV-2 S sequence is phenylalanine. In some embodiments, the residue other than tyrosine at the position corresponding to position 917 of the second SARS-COV-2 S sequence is tryptophan. In some embodiments, the residue other than threonine at the position corresponding to position 941 of the second SARS-COV-2 S sequence is asparagine or aspartate. In some embodiments, the residue other than alanine at the position corresponding to position 942 of the second SARS-CoV-2 S sequence is proline. In some embodiments, the residue other than asparagine at the position corresponding to position 955 of the second SARS-COV-2 S sequence is aspartate. In some embodiments, the residue other than alanine at the position corresponding to position 956 of the second SARS-COV-2 S sequence is leucine. In some embodiments, the residue other than lysine at the position corresponding to position 964 of the second SARS-COV-2 S sequence is glutamate. In some embodiments, the residue other than aspartate at the position corresponding to position 985 of the second SARS-COV-2 S sequence is asparagine. In some embodiments, the residue other than glutamate at the position corresponding to position 990 of the second SARS-COV-2 S sequence is arginine. In some embodiments, the residue other than glutamine at the position corresponding to position 1005 of the second SARS-COV-2 S sequence is methionine. In some embodiments, the residue other than threonine at the position corresponding to position 1009 of the second SARS-COV-2 S sequence is isoleucine. In some embodiments, the residue other than leucine at the position corresponding to position 1012 of the second SARS-COV-2 S sequence is glutamine. In some embodiments, the residue other than isoleucine at the position corresponding to position 1013 of the second SARS-COV-2 S sequence is leucine. In some embodiments, the residue other than alanine at the position corresponding to position 1016 of the second SARS-COV-2 S sequence is isoleucine. In some embodiments, the residue other than alanine at the position corresponding to position 1020 of the second SARS-COV-2 S sequence is leucine. In some embodiments, the residue other than asparagine at the position corresponding to position 1023 of the second SARS-COV-2 S sequence is lysine. In some embodiments, the residue other than serine at the position corresponding to position 1051 of the second SARS-COV-2 S sequence is threonine. In some embodiments, the residue other than proline at the position corresponding to position 1143 of the second SARS-COV-2 S sequence is glutamine or asparagine. Exemplary recombinant SARS-COV-2 S proteins comprising these modifications include Spk-M (SEQ ID NO:17), Spk-F (SEQ ID NO: 18), Spk-R (SEQ ID NO:19), and Spk-A (SEQ ID NO:20).

In some embodiments, the recombinant viral class I fusion proteins of the invention include a trimerization motif. The trimerization motif is preferably C-terminal to the “first” sequence of the recombinant viral class I fusion proteins (the first RSV F sequence, the first hMPV F sequence, or the first SARS-COV-2 S sequence). The trimerization motif is a motif that is configured to trimerize with itself to bring connected sequences in close proximity and, in some cases, interact with each other. A number of trimerization motifs are known in the art. These include the leucine zipper of the yeast transcriptional activator GCN4 factor¹⁵, the T4 bacteriophage fibritin domain¹⁶, the catalytic chain of aspartate transcarbamoylase (ATCase) from Escherichia coli¹⁷, or the trimer motifs MTQ and MTI¹⁸. Any of these trimerization motifs or variants thereof (e.g., sequences at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical thereto) can be used as a trimerization motif in the proteins of the invention. An exemplary trimerization motif provided herein includes positions 496-522 of SEQ ID NO:1 (GYIPEAPRDGQAYVRKDGEWVLLSTFL), and motifs comprising a trimerization sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to positions 496-522 of SEQ ID NO:1 (GYIPEAPRDGQAYVRKDGEWVLLSTFL).

The recombinant viral class I fusion proteins of the invention can further include any of number of other heterologous sequences. Exemplary sequences include linkers, affinity tags, or other catalytically active domains, among others. Linkers employed to fuse two heterologous polypeptides or domains to generate fusion proteins are well known in the art. See, e.g., U.S. Pat. Nos. 5,525,491, 6,274,331, 6,479,626, 10,526,379, 10,752,965, and 11,123,438, among others. Exemplary linkers include linkers comprising glycine and serine, such as a -G-S- linker or a -G-S-G- linker. Exemplary linker lengths can be from 1-20 residues in length, such as from 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 residues in length. Exemplary affinity tags include His tags, Strep II tags, T7 tags, FLAG tags, S tags, HA tags, c-Myc tags, dihydrofolate reductase (DHFR) tags, chitin binding domain tags, calmodulin binding domain tags, and cellulose binding domain tags. The sequences of each of these tags are well-known in the art. Preferred affinity tags are those smaller than about 20 amino acids. Other heterologous sequences include various cleavable linkers, (e.g., thrombin cleavage site: GLVPRGS; positions 523-530 of SEQ ID NOS: 1-4).

In some embodiments, the recombinant viral class I fusion proteins of the invention are soluble proteins. In such embodiments, the recombinant viral class I fusion proteins preferably comprise a trimerization domain as described above. In some embodiments, the recombinant viral class I fusion proteins of the invention are membrane proteins. In such embodiments, the recombinant viral class I fusion proteins preferably comprise a transmembrane domain or a membrane-binding domain. In embodiments in which the recombinant viral class I fusion proteins are membrane proteins, the recombinant viral class I fusion proteins are preferably expressed in or on a virus, a cell, a liposome, or another membrane-containing element.

Another aspect of the invention is directed to a recombinant polynucleotide encoding a recombinant viral class I fusion protein of the invention. The recombinant polynucleotide can comprise DNA or RNA. The recombinant polynucleotide can be in the form of a gene, a cDNA, an mRNA, a vector, etc.

Another aspect of the invention is directed to vaccines comprising the recombinant viral class I fusion proteins or polynucleotides of the invention. As used herein, “vaccine” refers to a composition that comprises the recombinant viral class I fusion proteins and/or polynucleotides of the invention and is capable of stimulating an immune response against the recombinant viral class I fusion proteins of the invention in a subject to whom the vaccine is administered. The vaccine can be in the form of an inactivated vaccine, a live-attenuated vaccine, a messenger RNA (mRNA) vaccine, a subunit vaccine, a recombinant vaccine, a conjugate vaccine, or a viral vector vaccine, among others. Methods for making vaccines such as these with a given antigen for RSV, hMPV, and SARS-COV-2 are well known in the art. See, e.g., U.S. Pat. No. 10,150,797, US 2013/0122032 A1, U.S. Pat. Nos. 11,338,031, 10,596,251, US 2021/0046173, U.S. Pat. Nos. 11,390,651, 11,478,517, 10,543,269, 10,543,269, US 2021/0228707, U.S. Pat. Nos. 11,241,493, 11,202,793, 11,471,525, 11,464,848, 11,497,807, 11,278,611, 11,406,703, 11,484,590, US 2021/0260182, US 2022/0184205, U.S. Pat. Nos. 11,298,417, 11,246,922, 11,103,576, US 2021/0246170, U.S. Pat. No. 11,473,064, US 2021/0346492, and US 2022/0331420, among others. The vaccines of the invention can comprise any component described in any one of these references.

Another aspect of the invention is directed to methods of vaccination. The methods can comprise administering a vaccine of the invention a subject in an amount effective to elicit an immune response against a viral class I fusion protein of the invention. The subject can be an animal, such as a mammal or human. The administration can comprise any suitable route. Exemplary routes include oral administration, intranasal administration, subcutaneous administration, and intramuscular administration, among others.

Unless otherwise indicated, the accession numbers referenced herein are derived from the NCBI database (National Center for Biotechnology Information) maintained by the National Institute of Health, U.S.A.

The term “altered property” refers to a modification in one or more properties of a mutant polynucleotide or mutant protein with reference to a corresponding polynucleotide or precursor protein.

The term “alignment” refers to a method of comparing two or more polynucleotides or polypeptide sequences for the purpose of determining their relationship to each other. Alignments are typically performed by computer programs that apply various algorithms, however it is also possible to perform an alignment by hand. Alignment programs typically iterate through potential alignments of sequences and score the alignments using substitution tables, employing a variety of strategies to reach a potential optimal alignment score. Commonly-used alignment algorithms include, but are not limited to, CLUSTALW, (see, Thompson J. D., Higgins D. G., Gibson T. J., CLUSTAL W: improving the sensitivity of progressive multiple sequence alignment through sequence weighting, position-specific gap penalties and weight matrix choice, Nucleic Acids Research 22: 4673-4680, 1994); CLUSTALV, (see, Larkin M. A., et al., CLUSTALW2, ClustalW and ClustalX version 2, Bioinformatics 23(21): 2947-2948, 2007); Jotun-Hein, Muscle et al., MUSCLE: a multiple sequence alignment method with reduced time and space complexity, BMC Bioinformatics 5: 113, 2004); Mafft, Kalign, ProbCons, and T-Coffee (see Notredame et al., T-Coffee: A novel method for multiple sequence alignments, Journal of Molecular Biology 302: 205-217, 2000). Exemplary programs that implement one or more of the above algorithms include, but are not limited to MegAlign from DNAStar (DNAStar, Inc. 3801 Regent St. Madison, Wis. 53705), MUSCLE, T-Coffee, CLUSTALX, CLUSTALV, JalView, Phylip, and Discovery Studio from Accelrys (Accelrys, Inc., 10188 Telesis Ct, Suite 100, San Diego, Calif. 92121). In a non-limiting example, MegAlign is used to implement the CLUSTALW alignment algorithm with the following parameters: Gap Penalty 10, Gap Length Penalty 0.20, Delay Divergent Seqs (30%) DNA Transition Weight 0.50, Protein Weight matrix Gonnet Series, DNA Weight Matrix IUB.

The term “consensus sequence” or “canonical sequence” refers to an archetypical amino acid sequence against which all variants of a particular protein or sequence of interest are compared. Either term also refers to a sequence that sets forth the nucleotides that are most often present in a polynucleotide sequence of interest. For each position of a protein, the consensus sequence gives the amino acid that is most abundant in that position in the sequence alignment.

The term “conservative substitutions” or “conserved substitutions” refers to, for example, a substitution of an amino acid with a conservative variant.

“Conservative variant” refers to residues that are functionally similar to a given residue. Amino acids within the following groups are conservative variants of one another: glycine, alanine, serine, and proline (very small); alanine, isoleucine, leucine, methionine, phenylalanine, valine, proline, and glycine (hydrophobic); alanine, valine, leucine, isoleucine, methionine (aliphatic-like); cysteine, serine, threonine, asparagine, tyrosine, and glutamine (polar); phenylalanine, tryptophan, tyrosine (aromatic); lysine, arginine, and histidine (basic); aspartate and glutamate (acidic); alanine and glycine; asparagine and glutamine; arginine and lysine; isoleucine, leucine, methionine, and valine; and serine and threonine.

“Corresponding proteins” as used herein refers to first and second proteins that are identical in sequence except for a given set of modifications. In some embodiments of the invention, any one or more modifications in the “first” sequences with respect to the “second” or “third” sequences can confer enhanced stability of a prefusion state of a recombinant viral class I fusion protein of the invention over a postfusion state compared to a corresponding protein not containing the one more modifications.

The terms “corresponds to” and “corresponding to” used with reference to an amino acid residue or position refer to an amino acid residue or position in a first protein sequence being positionally equivalent to an amino acid residue or position in a second reference protein sequence by virtue of the fact that the residue or position in the first protein sequence aligns to the residue or position in the reference sequence using bioinformatic techniques, for example, using the methods described herein for preparing a sequence alignment. The corresponding residue in the first protein sequence is then assigned the position number in the second reference protein sequence.

The term “deletion,” when used in the context of an amino acid sequence, means a deletion in or a removal of one or more residues from the amino acid sequence of a corresponding protein, resulting in a mutant protein having at least one less amino acid residue as compared to the corresponding protein. The term can also be used in the context of a nucleotide sequence, which means a deletion in or removal of a nucleotide from the polynucleotide sequence of a corresponding polynucleotide.

The term “derived” in the context of the recombinant viral class I fusion proteins of the invention being derived from native viral class I fusion proteins (e.g., the recombinant RSV F proteins being derived from the native respiratory syncytial virus (RSV) F protein, the recombinant human metapneumovirus (hMPV) F proteins being derived from the native human metapneumovirus (hMPV) F protein, and the recombinant SARS COV-2 S proteins being derived from the native SARS COV-2 S proteins) means that the recombinant proteins have at least some sequence identity to the corresponding native proteins and does necessarily imply any functional equivalence to the native counterparts. Thus, the recombinant viral class I fusion proteins of the invention are referred to as “class I fusion proteins” merely due their derivation from native class I fusion proteins and are not required to have any activity in viral fusion or any other specific functional characteristics of the natural counterparts unless explicitly specified herein. More specifically, the recombinant RSV F proteins, recombinant hMPV F proteins, and recombinant SARS COV-2 S proteins of the invention are not required to have any specific functional characteristics of their natural counterparts unless explicitly specified herein. Sequences derived from the RSV F protein are referred to herein as “RSV F” sequences. Sequences derived from the hMPV F protein are referred to herein as “hMPV F” sequences. Sequences derived from the SARS COV-2 S protein are referred to herein as “SARS-COV-2 S” sequences. The references to the sequences as such merely indicate the derivation of the sequences and do not require any particular structural identity or functional equivalence to the natural counterparts unless explicitly specified herein.

The term “DNA construct” is used herein to refer to a recombinant DNA that can be used, for example, to express a protein. Typically a DNA construct is generated in vitro by PCR or other suitable technique(s) known to those in the art. In certain embodiments, the DNA construct comprises a sequence of interest (e.g., an incoming sequence). In some embodiments, the sequence is operably linked to additional elements such as control elements (e.g., promoters, etc.). A DNA construct can further comprise a selectable marker. It can also comprise an incoming sequence flanked by homology targeting sequences. In a further embodiment, the DNA construct comprises other non-homologous sequences, added to the ends (e.g., stuffer sequences or flanks). In some embodiments, the ends of the incoming sequence are closed such that the DNA construct forms a closed circle. In some embodiments, the DNA construct comprises sequences homologous to the host cell chromosome. In other embodiments, the DNA construct comprises non-homologous sequences. Once the DNA construct is assembled in vitro it may be used to: 1) insert heterologous sequences into a desired target sequence of a host cell; 2) mutagenize a region of the host cell chromosome (i.e., replace an endogenous sequence with a heterologous sequence); 3) delete target genes; and/or (4) introduce a replicating plasmid into the host.

A polynucleotide is said to “encode” an RNA or a polypeptide if, in its native state or when manipulated by methods known to those of skill in the art, it can be transcribed and/or translated to produce the RNA, the polypeptide, or a fragment thereof. The antisense strand of such a polynucleotide is also said to encode the RNA or polypeptide sequences. As is known in the art, a DNA can be transcribed by an RNA polymerase to produce an RNA, and an RNA can be reverse transcribed by reverse transcriptase to produce a DNA. Thus a DNA can encode an RNA, and vice versa.

The term “express” can be used herein to refer to the production of an mRNA from a DNA, production of a protein from an mRNA, or production of a protein from a DNA via an mRNA.

The term “expressed genes” refers to genes that are transcribed into messenger RNA (mRNA) and then translated into protein, as well as genes that are transcribed into types of RNA, such as transfer RNA (tRNA), ribosomal RNA (rRNA), and regulatory RNA, which are not translated into protein.

The terms “expression cassette” or “expression vector” refer to a polynucleotide construct generated recombinantly or synthetically, with a series of specified elements that permit transcription of a particular polynucleotide in a target cell or in vitro. A recombinant expression cassette can be incorporated into a plasmid, chromosome, mitochondrial DNA, plasmid DNA, virus, or polynucleotide fragment. Typically, the recombinant expression cassette portion of an expression vector includes, among other sequences, a polynucleotide sequence to be transcribed and a promoter. In particular embodiments, expression vectors have the ability to incorporate and express heterologous polynucleotide fragments in a host cell. Many prokaryotic and eukaryotic expression vectors are commercially available. Selection of appropriate expression vectors is within the knowledge of those of skill in the art. The term “expression cassette” can be used interchangeably herein with “DNA construct,” and their grammatical equivalents.

“Gene” refers to a polynucleotide (e.g., a DNA segment) that encodes a polypeptide, and may include regions preceding and following the coding regions as well as intervening sequences (introns) between individual coding segments (exons).

The term “endogenous protein” refers to a protein that is native or naturally occurring. “Endogenous polynucleotide” refers to a polynucleotide that is in the cell and was not introduced into the cell using recombinant engineering techniques; for example, a gene that was present in the cell when the cell was originally isolated from nature.

The term “heterologous” used with reference to a protein or a polynucleotide in a host cell refers to a protein or a polynucleotide that does not naturally occur in the host cell. The term “heterologous” used to describe two different amino acid or nucleic acid sequences refers to two sequences that are not naturally present together in the same protein or nucleic acid. The term “heterologous” used to describe two different protein domains refers to two protein domains that are not naturally present together in the same protein. As used herein, “domain” refers to any portion of protein that confers a particular structural and/or functional characteristic to a protein. Exemplary protein domains include signal peptides, extracellular domains, transmembrane domains, cytoplasmic domains, catalytic domains, affinity tags, and linkers, among others.

The term “homologous sequences” as used herein refers to a polynucleotide or polypeptide sequence having, for example, about 100%, about 99% or more, about 98% or more, about 97% or more, about 96% or more, about 95% or more, about 94% or more, about 93% or more, about 92% or more, about 91% or more, about 90% or more, about 88% or more, about 85% or more, about 80% or more, about 75% or more, about 70% or more, about 65% or more, about 60% or more, about 55% or more, about 50% or more, about 45% or more, or about 40% or more sequence identity to another polynucleotide or polypeptide sequence when optimally aligned for comparison. In particular embodiments, homologous sequences can retain the same type and/or level of a particular activity of interest. In some embodiments, homologous sequences have between 85% and 100% sequence identity, whereas in other embodiments there is between 90% and 100% sequence identity. In particular embodiments, there is 95% and 100% sequence identity.

“Homology” refers to sequence similarity or sequence identity. Homology is determined using standard techniques known in the art (see, e.g., Smith and Waterman, Adv. Appl. Math., 2:482, 1981; Needleman and Wunsch, J. Mol. Biol., 48:443, 1970; Pearson and Lipman, Proc. Natl. Acad. Sci. USA 85:2444, 1988; programs such as GAP, BESTFIT, FASTA, and TFASTA in the Wisconsin Genetics Software Package (Genetics Computer Group, Madison, Wis.); and Devereux et al., Nucl. Acid Res., 12:387-395, 1984). A non-limiting example includes the use of the BLAST program (Altschul et al., Gapped BLAST and PSI-BLAST: a new generation of protein database search programs, Nucleic Acids Res. 25:3389-3402, 1997) to identify sequences that can be said to be “homologous.” A recent version such as version 2.2.16, 2.2.17, 2.2.18, 2.2.19, or the latest version, including sub-programs such as blastp for protein-protein comparisons, blastn for nucleotide-nucleotide comparisons, tblastn for protein-nucleotide comparisons, or blastx for nucleotide-protein comparisons, and with parameters as follows: Maximum number of sequences returned 10,000 or 100,000; E-value (expectation value) of 1e-2 or 1e-5, word size 3, scoring matrix BLOSUM62, gap cost existence 11, gap cost extension 1, may be suitable. An E-value of 1e-5, for example, indicates that the chance of a homologous match occurring at random is about 1 in 10,000, thereby marking a high confidence of true homology.

The term “host strain” or “host cell” refers to a suitable host for an expression vector comprising a DNA of the present invention. The host may comprise any organism, without limitation, capable of containing and expressing the nucleic acids or genes disclosed herein. The host may be prokaryotic or eukaryotic, single-celled or multicellular, including mammalian cells, plant cells, fungi, etc. Examples of single-celled hosts include cells of Escherichia, Salmonella, Bacillus, Clostridium, Streptomyces, Staphylococcus, Neisseria, Lactobacillus, Shigella, and Mycoplasma. Suitable E. coli strains (among a great many others) include BL21(DE3), C600, DH5aF′, HB101, JM83, JM101, JM103, JM105, JM107, JM109, JM110, MC1061, MC4100, MM294, NM522, NM554, TGI, χ1776, XL1-Blue, and Y1089+, all of which are commercially available.

The term “identical” (or “identity”), in the context of two polynucleotide or polypeptide sequences, means that the residues in the two sequences are the same when aligned for maximum correspondence, as measured using a sequence comparison or analysis algorithm such as those described herein. For example, if when properly aligned, the corresponding segments of two sequences have identical residues at 5 positions out of 10, it is said that the two sequences have a 50% identity. Most bioinformatic programs report percent identity over aligned sequence regions, which are typically not the entire molecules. If an alignment is long enough and contains enough identical residues, an expectation value can be calculated, which indicates that the level of identity in the alignment is unlikely to occur by random chance.

The term “insertion,” when used in the context of an amino acid sequence, refers to an insertion of an amino acid with respect to the amino acid sequence of a corresponding polypeptide, resulting in a mutant polypeptide having an amino acid that is inserted between two existing contiguous amino acids, i.e., adjacent amino acids residues, which are present in the corresponding polypeptide. The term “insertion,” when used in the context of a polynucleotide sequence, refers to an insertion of one or more nucleotides in the corresponding polynucleotide between two existing contiguous nucleotides, i.e., adjacent nucleotides, which are present in the corresponding polynucleotides.

The term “introduced” refers to, in the context of introducing a polynucleotide sequence into a cell, any method suitable for transferring the polynucleotide sequence into the cell. Such methods for introduction include but are not limited to protoplast fusion, transfection, transformation, conjugation, and transduction (see, e.g., Ferrari et al., Genetics, in Hardwood et al, (eds.), Bacillus, Plenum Publishing Corp., pp. 57-72, 1989).

The term “isolated” or “purified” means a material that is removed from its original environment, for example, the natural environment if it is naturally occurring, or a cultivation broth if it is produced in a recombinant host cell cultivation medium. A material is said to be “purified” when it is present in a particular composition in a higher concentration than the concentration that exists prior to the purification step(s). For example, with respect to a composition normally found in a naturally occurring or wild type organism, such a composition is “purified” when the final composition does not include some material from the original matrix. As another example, where a composition is found in combination with other components in a recombinant host cell cultivation medium, that composition is purified when the cultivation medium is treated in a way to remove some component of the cultivation, for example, cell debris or other cultivation products, through, for example, centrifugation or distillation. As another example, a naturally occurring polynucleotide or polypeptide present in a living animal is not isolated, but the same polynucleotide or polypeptide, separated from some or all of the coexisting materials in the natural system, is isolated, whether such process is through genetic engineering or mechanical separation. Such polynucleotides can be parts of vectors. Alternatively, such polynucleotides or polypeptides can be parts of compositions. Such polynucleotides or polypeptides can be considered “isolated” because the vectors or compositions comprising thereof are not part of their natural environments. In another example, a polynucleotide or protein is said to be purified if it gives rise to essentially one band in an electrophoretic gel or a blot.

The term “mutation” refers to, in the context of a polynucleotide, a modification to the polynucleotide sequence resulting in a change in the sequence of a polynucleotide with reference to a corresponding polynucleotide sequence. A mutation to a polynucleotide sequence can be an alteration that does not change the encoded amino acid sequence, for example, with regard to codon optimization for expression purposes, or that modifies a codon in such a way as to result in a modification of the encoded amino acid sequence. Mutations can be introduced into a polynucleotide through any number of methods known to those of ordinary skill in the art, including random mutagenesis, site-specific mutagenesis, oligonucleotide directed mutagenesis, gene shuffling, directed evolution techniques, combinatorial mutagenesis, and site saturation mutagenesis, among others.

“Mutation” or “mutated” means, in the context of a protein or polynucleotide, a modification to the protein or polynucleotide sequence resulting in a change in the sequence with respect to a corresponding sequence. A mutation can refer to a substitution of one residue (amino acid or nucleotide) with another residue, an insertion of one or more residues, or a deletion of one or more residues. A mutation can include the replacement of a naturally occurring residue with a non-natural residue, or a chemical modification of a residue. A mutation can also be a truncation (e.g., a deletion or interruption) in a sequence or a subsequence with respect to a corresponding sequence. A “mutant” as used herein is a protein or polynucleotide comprising a mutation.

A “naturally occurring equivalent,” in the context of the present invention, refers to a naturally occurring version of a protein or polynucleotide, e.g., a naturally occurring protein or polynucleotide from which a recombinant protein or polynucleotide is derived.

The term “operably linked,” in the context of a polynucleotide sequence, refers to the placement of one polynucleotide sequence into a functional relationship with another polynucleotide sequence. For example, a DNA encoding a secretory leader (e.g., a signal peptide) is operably linked to a DNA encoding a polypeptide if it is expressed as a preprotein that participates in the secretion of the polypeptide. A promoter or an enhancer is operably linked to a coding sequence if it affects the transcription of the sequence. A ribosome binding site is operably linked to a coding sequence if it is positioned so as to facilitate translation. Generally, “operably linked” means that the DNA sequences being linked are contiguous, and, in the case of a secretory leader, contiguous and in the same reading frame.

The term “optimal alignment” refers to the alignment giving the highest overall alignment score.

The terms “percent sequence identity,” “percent amino acid sequence identity,” “percent gene sequence identity,” and/or “percent polynucleotide sequence identity,” with respect to two polypeptides, polynucleotides and/or gene sequences (as appropriate), refer to the percentage of residues that are identical in the two sequences when the sequences are optimally aligned. Thus, 80% amino acid sequence identity means that 80% of the amino acids in two optimally aligned polypeptide sequences are identical. The percent identities expressed herein with respect to a given named reference sequence are determined over the entire reference sequence, rather than only a portion thereof. Thus, a first RSV F sequence that is at least 80% identical to positions 26-494 of a second RSV F sequence, for example, is at least about 80% identical to the entire sequence of positions 26-494 of a second RSV F sequence, as opposed merely to subsequences thereof.

The term “plasmid” refers to a circular double-stranded (ds) DNA construct used as a cloning vector, and which forms an extrachromosomal self-replicating genetic element in some eukaryotes or prokaryotes, or integrates into the host chromosome.

A “production host” is a cell used to produce products. As disclosed herein, a production host is modified to express or overexpress selected genes, and/or to have attenuated expression of selected genes. Non-limiting examples of production hosts include plant, animal, human, bacteria, yeast, cyanobacteria, algae, and/or filamentous fungi cells.

A “promoter” is a polynucleotide sequence that functions to direct transcription of a downstream gene. In preferred embodiments, the promoter is appropriate to the host cell in which the target gene is being expressed. The promoter, together with other transcriptional and translational regulatory polynucleotide sequences (also termed “control sequences”) is necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

The terms “protein” and “polypeptide” are used interchangeably herein. The 3-letter code as well as the 1-letter code for amino acid residues as defined in conformity with the IUPAC-IUB Joint Commission on Biochemical Nomenclature (JCBN) is used throughout this disclosure. It is also understood that a polypeptide may be coded for by more than one polynucleotide sequence due to the degeneracy of the genetic code. The terms “amino acid sequence” and “polypeptide sequence” are used interchangeably herein.

The terms “nucleic acid” and “polynucleotide” are used interchangeably herein.

The terms “recombinant,” used with respect to proteins and polynucleotides refers to protein and polynucleotides that are not found in nature, e.g., contain one or more mutations with respect to naturally occurring proteins and nucleic acids from which they are derived (naturally occurring counterparts). The term “recombinant,” when used to modify the term “cell” or “vector” herein, refers to a cell or a vector that comprises a recombinant protein or polynucleotide.

The terms “regulatory segment,” “regulatory sequence,” or “expression control sequence” refer to a polynucleotide sequence that is operatively linked with another polynucleotide sequence that encodes the amino acid sequence of a polypeptide chain to effect the expression of that encoded amino acid sequence. The regulatory sequence can inhibit, repress, promote, or even drive the expression of the operably-linked polynucleotide sequence encoding the amino acid sequence.

The term “substantially identical,” in the context of two polynucleotides or two polypeptides refers to a polynucleotide or polypeptide that comprises at least 70% sequence identity, for example, at least 75%, at least 80%, at least 85%, at least 90%, at least 91%, at least 92%, at least 93%, at least 94%, at least 95%, at least 96%, at least 97%, at least 98%, or at least 99% sequence identity as compared to a reference sequence using the programs or algorithms (e.g., BLAST, ALIGN, CLUSTAL) using standard parameters.

“Substantially purified” means molecules that are at least about 60% free, preferably at least about 75% free, about 80% free, about 85% free, and more preferably at least about 90% free from other components with which they are naturally associated. As used herein, the term “purified” or “to purify” also refers to the removal of contaminants from a sample.

“Substitution” means replacing an amino acid in the sequence of a corresponding protein with another amino acid at a particular position, resulting in a mutant of the corresponding protein. The amino acid used as a substitute can be a naturally occurring amino acid, or can be a synthetic or non-naturally occurring amino acid.

The term “transformed” or “stably transformed” cell refers to a cell that has a non-native (heterologous) polynucleotide sequence integrated into its genome or as an episomal plasmid that is maintained for at least two generations.

“Vector” refers to a polynucleotide construct designed to introduce polynucleotides into one or more cell types. Vectors include cloning vectors, expression vectors, shuttle vectors, plasmids, cassettes and the like. In some embodiments, the polynucleotide construct comprises a polynucleotide sequence encoding a protein that is operably linked to a suitable prosequence capable of effecting the expression of the polynucleotide or gene in a suitable host.

“Wild type” means, in the context of gene or protein, a polynucleotide or protein sequence, respectively, that occurs in nature. In some embodiments, the wild-type sequence refers to a sequence of interest that is a starting point for protein engineering. “Wild type” is used interchangeably with “native.”

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Stabilization of Viral Class I Fusion Proteins

### Summary

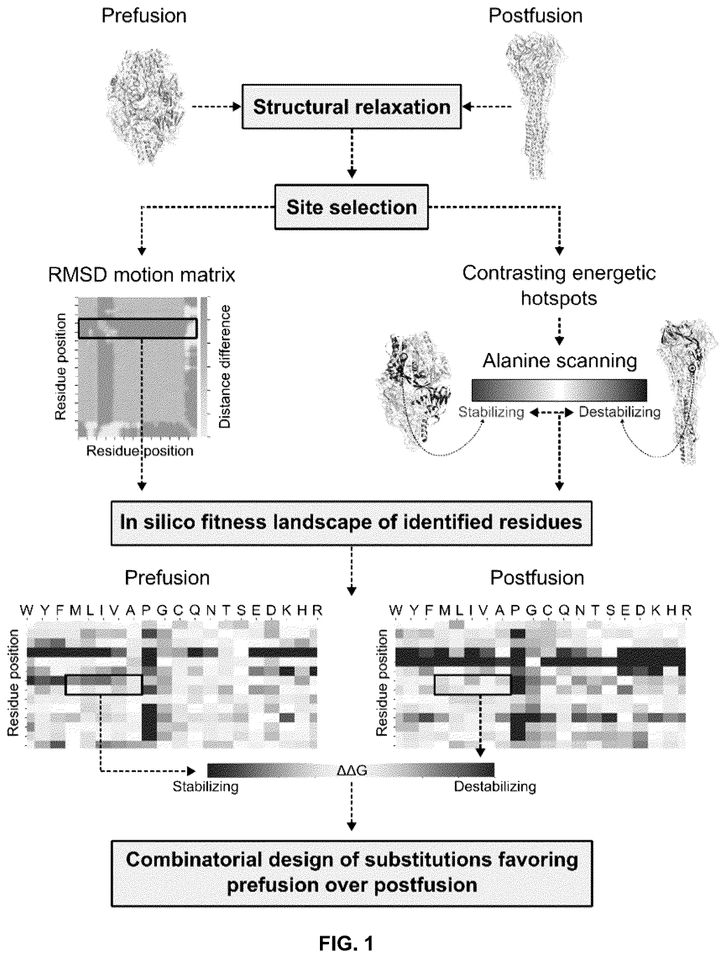

Many pathogenic viruses, including influenza virus, Ebola virus, coronaviruses, and Pneumoviruses, rely on class I fusion proteins to fuse viral and cellular membranes. To drive the fusion process, class I fusion proteins irreversibly refold from a metastable prefusion state to an energetically more favorable and stable postfusion state. We developed a general computational design strategy that takes advantage of knowing both the pre- and postfusion conformations to optimize the free energy of the prefusion state while destabilizing the other state (FIG. 1). Our approach offers a new means to stabilize viral surface proteins as vaccine candidates, as the prefusion conformation displays most of the epitopes targeted by potent neutralizing antibodies. As a proof of concept, we have stabilized the prefusion conformation of the RSV F, hMPV F, and SARS-COV-2 S proteins, using only 3-4 variants. Our models were designed at atomic accuracy as indicated by experimentally solved structures. The RSV F design was tested for its immunological response compared to a current clinical candidate in a mice model, and results showed an equivalent neutralizing antibody response. Given the clinical significance of viruses using class I fusion proteins, our method can substantially impact vaccine development by reducing the time and resources needed to optimize these potent immunogens and respond rapidly to emerging viral diseases.

### Introduction

Based on structural analyses of the fusion mechanism, the stabilization of the prefusion conformation has been mainly achieved by preventing the release of the fusion peptide or by disrupting the formation of the coiled-coil structure characteristic of the postfusion state¹⁹. Two strategies have been particularly successful by either designing disulfide bonds at regions undergoing remarkable refolding or introducing proline substitutions to impair the formation of the central postfusion helices^{13,20,21} Other stabilization methods have focused on identifying substitutions that increase favorable interactions or rigidify flexible areas in the prefusion structure. These methods either design cavity-filling substitutions^{13,21,22}, neutralize charge imbalances^{13,21,22}, or remove buried charged residues²³. While the strategies mentioned so far have been effective, the lack of an automated approach has limited the number of amino acid changes to be assessed and required extensive testing of variants. Notably, more than one hundred different protein variants were evaluated before finding a stable prefusion conformation of the Filovirus GP protein²⁴, the severe acute respiratory syndrome coronavirus 2 spike protein (SARS-COV-2 S)²⁵, and the F protein from Hendra²⁶, Nipah¹¹, respiratory syncytial virus (RSV F)^{21,22}, human metapneumovirus (hMPV F)²⁷, and parainfluenza virus types 1-4⁸.

To address these limitations, we developed a general computational approach where the fusion protein's sequence is optimized for the conformation of interest (here, the prefusion state) while destabilizing the other conformation. Our general strategy assumes that conformational rearrangements in class I fusion proteins can be frozen by introducing mutations that reduce the free energy of the prefusion form but do not benefit or better disrupt the postfusion state. While this “negative design” concept has been introduced before in multistate design (MSD) protocols²⁸, our efforts to implement leading algorithms in class I fusion proteins, such as the MPI_MSD²⁹, evidenced poor sequence sampling. This is likely due to the extensive sequence-structure search space that needs to be evaluated when modeling both states simultaneously of these large, underpacked proteins. Therefore, we simplified the design process by avoiding explicit negative design and using the undesired conformation as a guide to identify suboptimal positions for mutagenesis and their respective substitutions. In a second single-state combinatorial design step, we search for an optimal sequence for the conformation of interest within a subset of substitutions identified to improve the prefusion state while disfavoring the postfusion state. By applying the MSD concept in a single-state design protocol, we have successfully stabilized the prefusion state of large proteins, namely the RSV F, the hMPV F, and the SARS-COV-2 S proteins, illustrating its general use.

### Methods

### Computational Approach to Stabilize the Prefusion State

All computational analysis were performed with the Rosetta version: 2020.10.post.dev+12.master.c7b9c3e c7b9c3e4aeb1febab211d63da2914b119622e69b

### Structure Preparation

RSV F: All computational analyses were performed on the crystal structure of the RSV F protein in the prefusion (PDB:5w23)³⁰ and postfusion (PDB: 3rrt)³¹ conformations. To remove small clashes in the structures, both conformations were refined with the Rosetta relax application guided by electron density data³². Density maps were generated from the corresponding map coefficients files associated to the PDB accession codes. These coefficients were transformed into density maps using the Phenix software version 1.15³³ and the option “create map from map coefficients” (region padding=0, and grid resolution factor=0.3333). To include the electron density in the refinement process, the density energy term was activated in the Rosetta scoring function with a weight of 20. This weight was selected given the low-resolution of the density map and the starting structures. During the relaxation protocol, four rounds of rotamer packing and minimization were performed with gradual increases to the repulsive weight in the scoring function³⁴. After 5 cycles of relaxation, the quality of the resulting models was evaluated with the Molprobity web service³⁵. The structures with the lowest Rosetta energies and Molprobity scores were used for mutational analysis.

hMPV F: As described in the RSV F example, the hMPV F prefusion (PDB: 5wb0)²⁷ and postfusion (PDB: 511x)³⁶ conformations were relaxed using their respective electron density data. Due to the high resolution of the starting prefusion hMPV F structure (2.6 Å), the weight of the density energy term was increased to 50 to encourage a good agreement with the density map. All other parameters and postprocessing followed the RSV F example.

SARS-COV-2 S: The input SARS-COV-2 S prefusion and postfusion structures and their corresponding cryo-electron microscopy (cryo-EM) maps were retrieved with the PDB accession codes 6vxx³⁷ and 6xra³⁸, respectively. Since both structures were not completely solved, missing regions were modeled using the default comparative modeling protocol available in Rosetta³⁹. However, the cryo-EM density maps of the input pre- and postfusion structures were integrated in the modeling process to avoid large deviations from the original configuration. Templates selected for prefusion modeling corresponded to the PDB IDs 6m0j⁴⁰ and the initial 6vxx, while the postfusion structure was modeled using 6lxt⁴¹ and the initial 6xra. Model selection was based on overall agreement with the starting structure and templates, as well as a low Rosetta energy score. These homology models were then relaxed with the RosettaScripts⁴² framework, incorporating fit-to-density parameters established for cryo-EM density³². Due to the high resolution of the starting structures, the refinement process was performed with a density scoring weight of 50 and three cycles of FastRelax³⁴ in cartesian space. The structures with the lowest Rosetta energies and Molprobity scores were used for mutational analysis.

### Selection of Target Positions to Redesign

Residue positions to redesign were selected based on two independent approaches: I) contrasting energetic contributions to the pre- and postfusion conformations, and II) location on regions displaying drastic rearrangements between the pre- and postfusion states.

### Selection Based on Energetic Contributions

Amino acid positions with contrasting energetic contributions between conformations offer an opportunity to manipulate the energetics of the conformational switch by allowing the optimization of one state while the other state is disfavored. Identification of these target spots was done by in-silico alanine mutagenesis where the energetic role of each residue on each conformation was estimated using the change in folding energy upon mutation. Consequently, residue positions displaying simultaneous stabilization of the prefusion state and destabilization of the postfusion state were selected as hotspots to redesign. Details about the selection process are described at the computational alanine scanning section.

### Selection Based on Protein Dynamics

When Alanine Scanning was not Enough to Identify Positions with Contrasting Stability between conformations, all regions involved in the refolding process were chosen as targets to redesign. To identify these flexible areas, the root mean square deviation (RMSD) of each ca atom was calculated using their corresponding position in the pre- and postfusion structures. Both structures were structurally aligned prior to the analysis, and residue positions displaying motion levels of at least 10 Å were selected to redesign. Furthermore, residues flanking the highly movable areas were also considered to redesign when their secondary structure differed between the pre- and postfusion states. Flanking residues were included until a set of 8 (SARS-CoV-2 S) or 16 (hMPV F) consecutive residues matched their secondary structure in the pre- and postfusion structures.

### Computational Alanine Scanning

A computational alanine scanning was performed on the pre- and postfusion states of RSV F, hMPV F, and SARS-COV-2 S proteins to determine the energetic contributions of each amino acid to each conformation. Since the prefusion SARS-COV-2 S contains domains that are not present in the postfusion conformation, alanine scanning in this protein was limited to shared regions between states. Using the Rosetta ΔΔG protocol “cartesian_ddg”⁴³, the backbone and sidechains around the position to be mutated were optimized in the cartesian space and the change in folding energy (ΔG) was computed before and after each alanine substitution⁴³. The contribution of every residue to stability was calculated in terms of ΔΔG scores (ΔGₘᵤₜₐₙₜ Δ-G_{wild type}) where alanine changes holding ΔΔG scores<−1.0 were considered stabilizing substitutions while ΔΔG scores>1.0 were considered destabilizing changes⁴³. All calculations were repeated 3 times and the average value among repetitions was used as the final ΔΔG score. To enhance the prefusion stability over the postfusion, regions presenting a stabilizing score in the prefusion conformation but not in postfusion conformation were chosen to redesign. Specifically, designable positions were selected based on a) stabilizing ΔΔG score in the prefusion conformation and destabilizing ΔΔG score in the postfusion conformation, b) stabilizing ΔΔG score in the prefusion conformation and neutral ΔΔG score in the postfusion conformation, or c) destabilizing ΔΔG score in the postfusion conformation and neutral ΔΔG score in the prefusion conformation. To restrict the design process to the most relevant spots, positions meeting any of the above criteria were filtered based on an energetic difference of at least 0.7 (ΔΔG_{postfusion}−ΔΔG_{prefusion}). Finally, since positions with native alanine are overlooked with this approach, all alanine-bearing spots were also included as targets to redesign.

### Computational Protein Design

To determine the amino acid identities likely to invert the energetics of the pre- and postfusion states, target positions to redesign underwent complete in-silico saturation mutagenesis as described at the computational alanine scanning section. Subsequently, substitutions favoring the prefusion state over the postfusion state were chosen for combinatorial design through Rosetta modeling. To bias the design process towards mutations displaying preference for the prefusion state with a high energetic difference between states, the weight of each substitution was adjusted in the Rosetta energy function according to a fitness score. Our fitness score compiled the stabilization effect of one mutation in both pre- and postfusion states by subtracting the ΔΔG prefusion score from the ΔΔG postfusion score (ΔΔG_{postfusion}−ΔΔG_{prefusion}). Mutations favoring the prefusion state over the postfusion conformation were then characterized by positive fitness scores where higher values represented bigger energetic gaps between states. The fitness score was incorporated into the Rosetta score function through a residue-type constraint term derived from the “FavorSequenceProfile” mover. Since this mover was initially created to re-weight amino acid substitutions depending on their occurrence in a multiple sequence alignment, we have replaced the original position-specific substitution matrix (PSSM) input by a fitness score matrix. To follow the PSSM format, negative fitness scores were replaced by zero and a 0.05 pseudocount was used for the log-odds scores calculation. After tuning the profile weights of each residue, “allowed” mutations at every amino acid position were defined as those with a fitness score greater than or equal to 0.7. For challenging targets such as the SARS-COV-2 S, the threshold difference was increased to 2 to focus on the most significant substitutions. Likewise, beneficial mutations for both states were allowed only if the stabilization effect in the prefusion state was at least 4 units greater than in the postfusion state. Finally, the combinatorial sequence design was carried out by the FastDesign algorithm^{44,45}. Upon conclusion, further optimization of a specific target spot was optionally done by applying FastDesign (all amino acids allowed) on residues neighboring 6 Å around the point of interest and limiting packing and minimization to a 12 Å sphere. The design process was initially performed on the prefusion conformation, and the resulting sequences were modeled on the postfusion structure for energetic comparisons.

### Selection of Top Designs

Promising designs were first sorted based on their Rosetta total energy score. Before comparison, the parent pre and postfusion structures were relaxed and energetically minimized using the same protocol as the designed models. Top candidates corresponded to designs showing a lower energy score in the prefusion state compared to the parent pre- and postfusion conformations. Analogously, the designed sequence in the postfusion state had to display a higher energy score than the parent postfusion conformation. The selected designs were then analyzed at the residue level to identify which mutations contributed more to the energetic gap between states. In this regard, mutations were filtered according to different structural metrics, such as Rosetta per-residue energy, and the number of hydrogen bonds and van der Waals contacts. To improve the prefusion state over the postfusion conformation, mutations showing favorable metrics on the prefusion state and unfavorable values for the postfusion conformation were selected to be tested experimentally.

### Exemplary Step-Wise Protocol

An exemplary protocol for the analysis is as follows:
1. Perform alanine scanning with standard “Cartesian ddg” protocol on every residue of the prefusion and postfusion structures.
2. Compare pre vs post alanine ddg results and perform all-amino acids scanning on positions stabilizing the prefusion structure over the postfusion structure. Alternatively, perform all-amino acids scanning on regions undergoing drastic conformational changes between pre- and postfusion structures.
3. Compare pre vs post all-amino acids ddg results and select positions for combinatorial design based on stabilization of the prefusion structure over the postfusion structure.
4. Perform combinatorial design in the prefusion state.
5. Select sequences where the prefusion total energy improved and model the sequences in the postfusion structure.
6. Identify sequences where the total energy of the prefusion structure improved while the postfusion structure got worse.
7. From sequences where prefusion improved over postfusion, check per-residue energy of each mutation in the prefusion structure and select positions where the mutation energy improved compared to the wild-type energy.

From here, the “interesting mutations” were checked manually, and the design process was repeated with selected positions based on the manual inspection.

### Protein Expression and Characterization

### Protein Expression

The top 3-4 RSV F, hMPV F, and SARS-COV-2 S computational designs as well as the starting constructs hMPV F 115-BV²⁷, and SARS-COV-2 S-2P⁴⁶, and the control variants RSV F DS-Cav1¹³, postfusion RSV A2 F⁴⁷, and postfusion hMPV B2 F⁴⁸ were expressed by transient transfection of FreeStyle 293-F cells (Thermo Fisher) with polyethylenimine (PEI) (Polysciences). All computationally designed variants were produced in pCAGGS plasmids encoding the sequence of interest, a C-terminal T4 fibritin trimerization motif (Foldon), and a His6-tag. The designed RSV F constructs contained residues 1-105 and 137-513, and a flexible linker replacing the furin cleavage sites and p27 peptide (“QARGSGSGR”-positions 106-114 of SEQ ID NOS: 1-4)²¹. Likewise, the designed hMPV F sequences included residues 1-95 and 103-472, a modified cleavage site “ENPRRRR” (positions 96-102 of SEQ ID NOS:7-15), and the A185P mutation²⁷. Finally, the designed SARS-COV-2 S variants followed the semi-stabilized SARS-COV-2 S-2P protein sequence⁴⁶, with two proline substitutions at residues 986 and 987, and a “GSAS” linker replacing the furin cleavage site. All DNA sequences were codon optimized for human expression using the online tool GenSmart Codon Optimization⁴⁹, and included a Kozak sequence (GCTAGCGCCACC, SEQ ID NO:28) upstream of the coding sequence. Cells were cultured at 37° C. and 8% CO₂, and the culture supernatant was harvested on the 3rd day after transfection. Proteins were purified by nickel affinity chromatography followed by size-exclusion chromatography (SEC) in phosphate-buffered saline (PBS) buffer pH 7.4. RSV F and hMPV F variants were SEC purified using a Superdex200 column (Cytiva) while SARS-COV-2 S was purified with a Superdex6 column (Cytiva). The sequences of various constructs are outlined in Table 1, and the features of the sequences are outlined in Table 2.

The angiotensin-converting enzyme 2 (ACE2) was expressed as an Fc-fusion⁵⁰ after transient transfection as described above. The protein was purified using a Protein A agarose gravity column (Millipore Sigma) followed by SEC using a S200 column.

TABLE 1
Sequences of constructs.

| | | SEQ ID |
|---|---|---|
| Viral Protein | Construct | NO* |
| RSV F Protein | R-Base | 1 |
| | R-1b | 2 (21) |
| | R-02 | 3 |
| | R-03 | 4 |
| | R-Base-FL | 5 |
| | R-1b-FL | 6 |
| hMPV F Protein | M-Base | 7 |
| | M-102 | 8 |
| | M-104 | 9 (22) |
| | M-302 | 10 |
| | M-305 | 11 (23) |
| | M-374 | 12 |
| | M-404 | 13 |
| | M-503 | 14 |
| | M-601 | 15 |
| SARS-COV-2 S | Spk-Base | 16 |
| Protein | Spk-M | 17 (24) |
| | Spk-F | 18 (25) |
| | Spk-R | 19 (26) |
| | Spk-A | 20 (27) |

*Numbers in parentheses indicate SEQ ID NOS of coding sequences.

TABLE 2
Sequence features.

| SEQ | | | |
|---|---|---|---|
| ID | | | |
| NOS: | Positions | Sequence | Feature |
| 1-4 | 1-25 | MELLILKANAITTILTAVTFCFASG | Signal peptide |
| | 106-115 | QARGSGSGRS | Modified |
| | | | cleavage site |
| | 495 | G | Linker |
| | 496-522 | GYIPEAPRDGQAYVRKDGEWVLLSTFL | Foldon (T4 |
| | | | fibritin |
| | | | trimerization |
| | | | motif) |
| | 523-530 | GGLVPRGS | Thrombin site |
| | 531-536 | HHHHHH | His6-tag |
| 5-6 | 1-25 | MELLILKANAITTILTAVTFCFASG | Signal peptide |
| | 517 | G | Linker |
| | 518-544 | GYIPEAPRDGQAYVRKDGEWVLLSTFL | Foldon (T4 |
| | | | fibritin |
| | | | trimerization |
| | | | motif) |
| | 545-552 | GGLVPRGS | Thrombin site |
| | 553-558 | HHHHHH | His6-tag |
| 7-15 | 1-18 | MSWKVVIIFSLLITPQHG | Signal peptide |
| | 91-102 | REEQIENPRRRR | Modified |
| | | | cleavage site |
| | 490-491 | GS | Linker |
| | 492-518 | GYIPEAPRDGQAYVRKDGEWVLLSTFL | Foldon (T4 |
| | | | fibritin |
| | | | trimerization |
| | | | motif) |
| | 519-524 | HHHHHH | His6-tag |
| 16-20 | 1-14 | MFVFLVLLPLVSSQ | Signal peptide |
| | 682-685 | GSAS | Modified |
| | | | cleavage site |
| | 1209-1210 | GS | Linker |
| | 1211-1237 | GYIPEAPRDGQAYVRKDGEWVLLSTFL | Foldon (T4 |
| | | | fibritin |
| | | | trimerization |
| | | | motif) |
| | 1238-1243 | HHHHHH | His6-tag |

### Antigenic Characterization

Bio-layer interferometry (BLI) was used to evaluate the structural and antigenic conservation of prefusion-specific epitopes. The prefusion-specific binders used for this purpose were the antibodies D25^{51,52} (Thermo Fisher) and AM14^{52,53} (Cambridge Biologics) for RSV F, MPE8⁵⁴ and 465⁵⁵ for hMPV, and the angiotensin-converting enzyme 2 (ACE2)⁵⁰ for SARS-COV-2 S. All binders were immobilized on Protein A sensors (GatorBio) at a concentration of 15 nM (RSV F, and hMPV F), or 40 nM (SARS-COV-2 S). Binding against expressed designs was tested at eight different protein concentrations starting from 200 nM (RSV F, and hMPV F) or 400 nM (SARS-COV-2 S) and decreasing by 1:2 dilutions. All solutions had a final volume of 200 μL/well using PBS buffer supplemented with 0.02% tween-20 and 0.1 mg/mL bovine serum albumin (BLI buffer). Biosensor tips were equilibrated for 20 min in BLI buffer before loading of binders. Loading was then carried out for 180s followed by a baseline correction of 120s. Subsequently, association and dissociation between the binders and designed variants were allowed for 180s each. To validate the BLI results, binding with previous prefusion-stabilized proteins such as the RSV F DS-Cav1¹³, hMPV F 115-BV²⁷, and SARS-COV-2 S-2P⁴⁶ was used as positive controls, and binding with the postfusion constructs RSV A2 F⁴⁷, and hMPV B2 F⁴⁸ was used as negative controls. All assays were performed using a GatorPrime biolayer interferometry instrument (GatorBio) at a temperature of 30° C. and frequency of 10 Hz. Data analysis was completed with the GatorOne software 1.7.28, using a global association model 1:1.

### Thermal Stability

The thermal stability of the expressed variants was assessed by differential scanning fluorimetry (DSF). The samples were prepared by creating a solution containing 1.2 μL SYPRO orange fluorescent dye (Thermo Fisher) with 3 μL of 100 mM MgCl₂, 3 μL of 1M KCl, and 3 μL of 1M Tris (pH 7.4). The final solution volume was 60 μL with a protein concentration of 3.5 μM. A negative sample with no protein was also prepared as background control. All measurements were performed by triplicates using 20 μL of sample. The data was collected with a qPCR instrument (CFX Connect, BioRad) and a temperature ramp from 25 to 90° C. with 0.5° C. increments. The melting temperature was determined based on the lowest point of the negative first derivative of the SYPRO Orange signal.

Antigenic preservation in variants displaying the highest melting temperature was further evaluated after one hour incubation at 55 and 60° C. This process was carried out in a thermocycler with heated lid (T100, BioRad). The conservation of the antigenic sites was determined by binding to prefusion-specific binders as described at the antigenic characterization section. Conversion to the postfusion state was also evaluated for the RSV F variant R-1b using the postfusion-specific antibody 131-2A⁵⁶ (Millipore Sigma) and the postfusion RSV A2 F⁴⁷ as positive control.

### Negative-Stain Electron Microscopy

Purified R-1b, M-104, and Spk-M (buffer-exchanged into 50 mM Tris pH 7.5 and 100 mM NaCl) were applied on a carbon-coated copper grids (400 mesh, Electron Microscopy Sciences) using 5 μL of protein solution (10 μg/mL) for 3 min. The grid was washed in water twice and then stained with 0.75% uranyl formate (R-1b) or Nano-W (Nanoprobes) (M-104, and Spk-M) for 1 min. Negative-stain electron micrographs were acquired using a JEOL JEM1011 transmission electron microscope equipped with a high-contrast 2K-by-2K AMT mid-mount digital camera.

### X-Ray Crystallization

The trimeric R-1b and M-104 proteins were concentrated to 14 mg/mL and 13.9 mg/mL, respectively, and crystallization trials were prepared on a TTP LabTech Mosquito Robot in sitting-drop MRC-2 plates (Hampton Research) using several commercially available crystallization screens. R-1b crystals were obtained in the Index HT (Hampton Research) in condition H6 (0.2 M Sodium formate, 20% w/v PEG 3,350), while M-104 crystals were obtained in the Crystal screen (Hampton research) in condition C10 (0.1 M Sodium acetate trihydrate pH 4.6, 2.0 M Sodium formate). Crystals were harvested and cryo-protected with 30% glycerol in the mother liquor before being flash frozen in liquid nitrogen. X-ray diffraction data were collected at the Advanced Photon Source SER-CAT beamLine 21-ID-D. Data were indexed and scaled using XDS⁵⁷. A molecular replacement solution was obtained in Phaser³³ using the prefusion RSV F SC-TM structure (PDB 5c6b)²¹ or the prefusion hMPV F 115-BV (PDB 5wb0)²⁷. The crystal structures were completed by manually building in COOT⁵⁸ followed by subsequent rounds of manual rebuilding and refinement in Phenix³³. The data collection and refinement statistics are shown in Table 3.

TABLE 3
Data collection and refinement statistics for R-1b and M-104.

| | R-1b | M-104 |
|---|---|---|
| PDB ID | 7TN1 | 8E15 |
| Wavelength | 1 Å | 1 Å |
| Resolution range | 49.3-3.1 (3.211-3.1) | 47.62-2.41 (2.496-2.41) |
| Space group | P 41 21 2 | I21 3 |
| Unit cell | 170.5 170.5 171.2 90 90 90 | 178.191 178.191 178.191 90 90 90 |
| Total reflections | 286706 (32084) | 72725 (7212) |
| Unique reflections | 44788 (4518) | 36363 (3606) |
| Multiplicity | 6.4 (7.2) | 2.0 (2.0) |
| Completeness (%) | 96.0 (99.47) | 99.91 (99.86) |
| Mean I/sigma(I) | 6.3 (1.7) | 9.18 (0.78) |
| Wilson B-factor | 78.2 | 66.92 |
| R-merge | 0.268 (1.34) | 0.03633 (0.921) |
| R-meas | 0.292 (1.44) | 0.05138 (1.302) |
| R-pim | 0.113 (0.523) | 0.03633 (0.921) |
| CC1/2 | 0.981 (0.518) | 0.999 (0.593) |
| CC* | 0.995 (0.826) | 1 (0.863) |
| Reflections used in | 44449 (4513) | 36339 (3601) |
| refinement | | |
| Reflections used for | 1986 (208) | 1871 (192) |
| R-free | | |
| R-work | 0.257 (0.297) | 0.2036 (0.3056) |
| R-free | 0.315 (0.371) | 0.2487 (0.3366) |
| Number of non- | 10472 | 3438 |
| hydrogen atoms | | |
| Macromolecules | 10420 | 3360 |
| Ligands | 42 | 67 |
| Solvent | 10 | 11 |
| Protein residues | 1361 | 443 |
| RMS (bonds) | 0.010 | 0.009 |
| RMS (angles) | 1.29 | 1.02 |
| Ramachandran | 92.3 | 97.27 |
| favored (%) | | |
| Ramachandran | 7.5 | 2.73 |
| allowed (%) | | |
| Ramachandran | 0.22 | 0.00 |
| outliers (%) | | |
| Rotamer outliers (%) | 0.25 | 1.08 |
| Clashscore | 13.1 | 6.61 |
| Average B-factor | 71.2 | 70.90 |
| Macromolecules | 71.1 | 70.11 |
| Ligands | 109.5 | 110.72 |
| Solvent | 30.0 | 70.94 |

Statistics for the highest-resolution shell are shown in parentheses.

### Cryo-Electron Microscopy

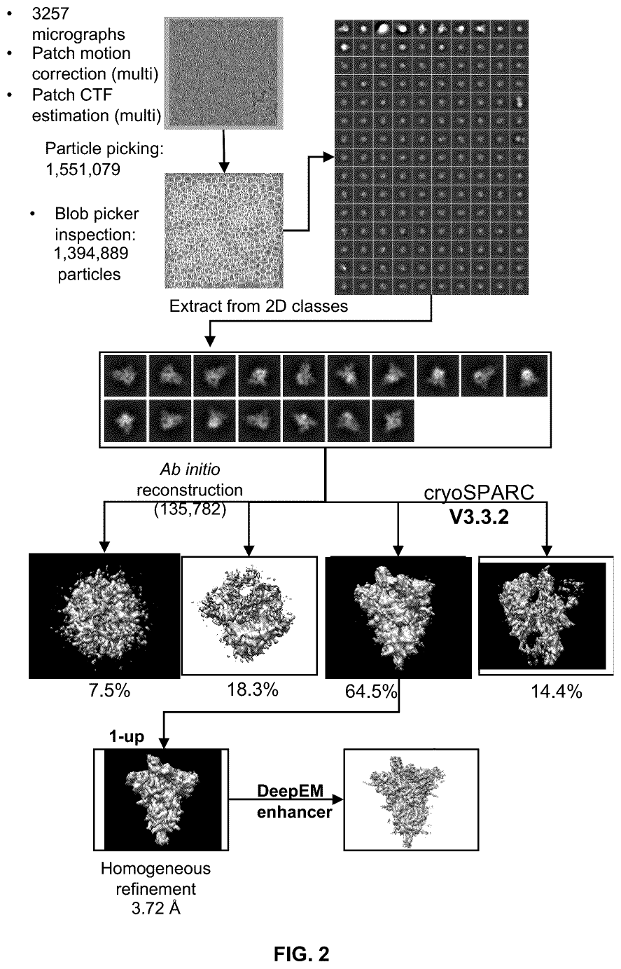

Spk-M cryo-EM density data was obtained by the Eyring Materials Center at Arizona State University (ASU). Purified protein was diluted to a concentration of 0.35 mg/mL in Tris-buffered saline (TBS) and applied to plasma-cleaned CF-300 2/1 grids before being blotted for 3 seconds in a Vitrobot Mark IV (Thermo Fisher) and plunge frozen into liquid ethane. 3,257 micrographs were collected from a single grid using a FEI Titan Krios (Thermo Fisher) equipped with a K2 summit direct electron detector (Gatan, Pleasantville CA.). Data collection was automated with SerialEM with a defocus range of −0.8 to −2.6 um in counting mode on the camera with a 0.2 second frame rate over 8 seconds and total dose of 58.24 electron per angstrom squared. Images were processed using cryoSPARC V3.3.2⁵⁹ (FIG. 2). Micrographs were patch motion corrected. After particle extractions, the blob picker was used, and 1,551,079 and picking was manually adjusted to reduce “blobs” to 1,394,889 particles. After 2D classifications first 4 classes were ab initio reconstructed and heterogeneously refined. The most populated map was refined with homogenous refinement in cryoSPARC, resulting in a 3.72 Å map. The map was further processed in DeepEM⁶⁰. The resulting final map was aligned with a previously published SARS-COV-2 S with one RBD domain up (PDB ID 6vyb³⁷) using UCSF Chimera-1.15⁶¹. Mutations and coordinate fitting were done manually using COOT⁵⁸ and structure optimization was achieved by iterative refinement using Phenix real space refinement³³ and COOT. The model and map statistics are presented in Table 4.

TABLE 4
Cryo-EM data collection and refinement statistics for Spk-M.

| | Spk-M |
|---|---|
| PDB ID | 8FEZ |
| EMDB | EMD-29035 |
| Microscope | Krios |
| Magnification | 22,500 |
| Pixel size (Å) | 1.024 |
| Voltage (kV) | 300 |
| Electron exposure (e−/Å2) | 58.24 |
| Frames | 8 sec exposure, 0.2 sec frames, |
| | 40 total frames |
| Defocus Range (μm) | −0.8 to −2.6 |
| Number of movies collected | 3257 |
| Particles extracted | 1,394,889 |
| Symmetry | C1 |
| Map resolution (Å) | 3.72 |
| Protein residues | 2843 |
| RMS (bonds) | 0.018 |
| RMS (angles) | 1.749 |
| Ramachandran favored (%) | 95.40 |
| Ramachandran allowed (%) | 4.60 |
| Ramachandran outliers (%) | 0.00 |
| Rotamer outliers (%) | 0.22 |
| Clashscore | 7.96 |

### Animal Studies (RSV Only)

### Mice Immunizations

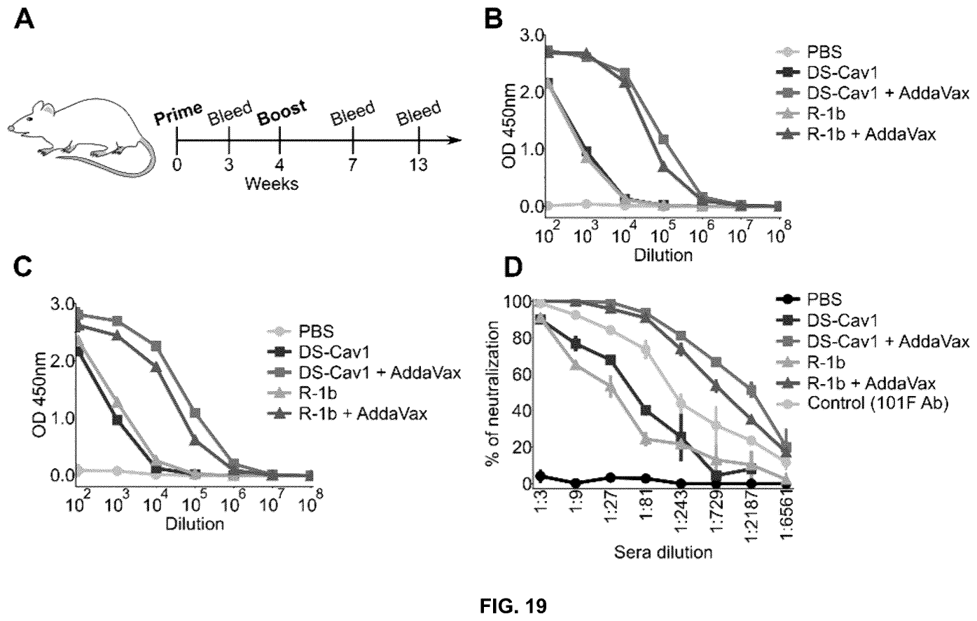

All animal experiments were performed in accordance with the guidelines and approved protocols by the Institutional Animal Care and Use Committee at University of Georgia, Athens, USA. Six-to-eight-week female BALB/c mice were purchased and housed in microisolator cages in the animal facility at University of Georgia. Food and water were provided ad libitum. After acclimation period, 5 mice per group were intramuscularly (i.m.) inoculated with total 100 μL of two different doses (2 μg and 0.2 μg) of either purified DS-Cav1 or R-1b protein with AddaVax adjuvant (50% v/v) or PBS at weeks 0 and 4 (Prime and Boost Vaccination protocol) (FIG. 19(A)). Bleeds were collected from tail vein pre- and post-immunization (3, 7, and 13 weeks) and sera were analyzed by ELISA and neutralization assay.

### Measurement of IgG Response by ELISA

Medium binding 96 wells microplates (Greiner Bio-One) were coated with 50 μL per well of DS-Cav1 or R-1b protein at 2 μg/mL at 4° C. overnight. Plates were washed in PBS/0.05% Tween 20 (Promega) and then blocked with blocking buffer solution (PBS/0.05% Tween 20/3% non-fat milk (AmericanBio)/0.5% Bovine Serum Albumin (Sigma)) at room temperature for 2 hours. Pooled serum from each group of mice pre- and post-different stages of immunization or control were inactivated at 56° C. per 1 hour for subsequent serial dilution in blocking buffer. 100 μL per well of inactivated diluted sera were incubated in triplicate at room temperature for 2 hours. Subsequently, three washes were performed, and plates were incubated with peroxidase-labeled goat anti-mouse IgG (1:3500) (SeraCare) diluted in blocking buffer. After one-hour incubation at room temperature, plates were washed and TMB substrate working Solution (Vector Laboratories) was added. After 10 min at room temperature, the reaction was stopped by adding 50 μL per well of Stop Solution for TMB ELISA (1N H2SO4). Plates were then read on Cytation7 imaging Reader (BioTek) at 450 nm.

### RSV Neutralization Assays

Pooled serum samples from mice in each immunization group (5 animals/group) after vaccination and boost (13 weeks after the beginning of the experiment or prime vaccination/9 weeks after Prime and Boost vaccination) were diluted in Opti-MEM media (Thermofisher) in serial 3-fold dilutions. Antibody 101F (provided by Jarrod J. Mousa) was used as a positive control for virus neutralization starting at 20 μg/mL. Further, dilutions were mixed with 120 focus-forming units (FFU) of RSV A virus (strain: rA2 line19F) (kindly provided by Dr. Martin Moore) and incubated for 1 hour at room temperature. Subsequently, RSV and sera/antibody dilutions were added to Vero E6 (ATCC) monolayer (105 cells/well) in triplicate and incubated for one hour at 37° C., gently rocking the plate every 15 minutes. Following the incubation, cell monolayers were covered with an overlay of 0.75% methylcellulose dissolved in Opti-MEM with 2% Fetal Bovine Serum (FBS) (Thermofisher) and incubated at 37° C., 5% CO2. After four days, the overlay was removed, and wells fixed with neutral buffered formalin 10% (Sigma) at room temperature for 30 minutes. Further, fixed monolayers were washed with water and dried at room temperature. An FFU assay was performed to identify the percentage of RSV neutralization. Briefly, wells were washed gently with PBS-0.05% Tween-20 (Promega) and incubated per one hour with anti-RSV polyclonal antibody (EMD Millipore) diluted 1:500 in dilution buffer [5% Non-fat dry milk (AmericanBio) in PBS-0.05% Tween-20]. Plates were washed three times with PBS-0.05% Tween-20, followed by 30 minutes incubation of secondary antibody HRP conjugate rabbit anti-goat IgG (Millipore Sigma) diluted 1:500 in dilution buffer. After incubation, wells were washed, and TMB Peroxidase substrate (Vector Laboratories) was added for 1 hour at room temperature. The visualized foci per well were counted under an inverted microscope.

### Results

### Energy Optimization of the Prefusion Over the Postfusion Conformation

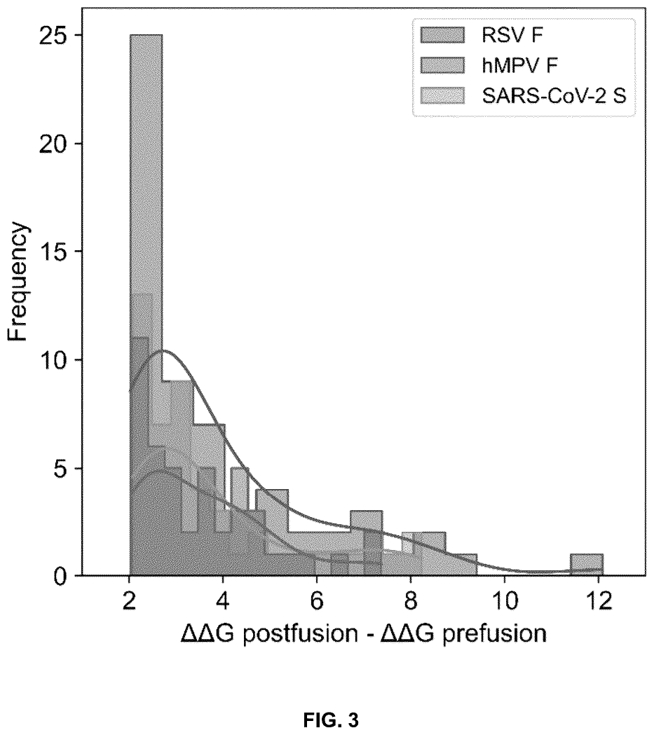

Fusion proteins must contain various energetically sub-optimal residues for a given conformation to be able to accommodate other conformations required to complete the fusion process⁶. As a first step, we identified these sub-optimal positions for the prefusion conformation based on the protein energetics or its anticipated dynamics (FIG. 1). For the first approach, mainly used for the stabilization of the RSV F protein (based on the A2 strain, as published under the PDB 5w23³⁰), we uncovered residue positions with contrasting stability between the pre- and postfusion conformations by calculating the energetic contribution of every residue to each state. In-silico alanine mutagenesis allowed us to quickly identify contributions towards Gibbs free energy (ΔΔG) of a given residue side chain, approximating the role of the position on the stability of each state⁴³. Negative ΔΔG scores (<−1.0 in Rosetta energy units, REU) indicated structural stabilization while positive ΔΔG scores (>1.0 REU) suggested destabilization⁴³. Thereby, we created two energetic maps, one for each state, to spotlight residue positions with differential contributions on each conformation (FIG. 1). In all our examples, about 40-50 positions displayed higher stability in the prefusion state than in the postfusion state. However, RSV F presented the biggest energetic differences between states when compared to our other examples (the hMPV F and SARS COV-2 S proteins) (FIG. 3).

When alanine scanning was not sufficient to locate meaningful designable spots, as defined by a differential of at least 2 REU, we focused on the dynamics of the protein as a second approach to identify sub-optimal positions. For hMPV F and SARS-COV-2 S proteins, we defined as “designable” all regions undergoing drastic structural rearrangements between states. Highly movable residues and all positions identified by alanine scanning were exhaustively explored to find substitutions that invert the energetics between states.

The following mutations were predicted to confer higher stability of the prefusion state of the RSV F constructs (SEQ ID NOS:1-4) than the postfusion state (the positions in parentheses refer to the numbering in the full-length RSV F constructs (SEQ ID NOS:5-6) and can be implemented therein to confer its prefusion stability; the underlined mutations are those that appear in the combinatorial variants as described below): S55A, S55I, S55L, S55V, E60F, E60I, N70Q, Q94R, M97I, M97T, S128E (S150E), S128L (S150L), G129A (G151A), N153R (N175R), Q203E (Q225E), N205F (N227F), N205L (N227L), V256L (V278L), S308D (S330D), T315L (T337L), E356K (E378K), N358K (N380K), E465N (E487N), and E465V (E487V).

The following mutations were predicted to confer higher stability of the prefusion state of the hMPV F constructs (SEQ ID NOS:7-15) than the postfusion state (the underlined mutations are those that appear in the combinatorial variants as described below): G42D, A90N, G106F, G106R, G106W, A107F, A107L, T114E, A116V, L130D, K143D, S149I, S149T, T150D, G152K, A159I, A159L, T160F, V162I, L187I, V191I, V203I, D209E, A216R, A216S, S237H, G277D, G277E, G277K, A314K, A314N, S371P, G393S, V430Q, 1437R, V449D, V449E, and E453P.

The following mutations were predicted to confer higher stability of the prefusion state of the SARS-COV-2 S constructs (SEQ ID NOS: 16-20) than the postfusion state (the underlined mutations are those that appear in the combinatorial variants as described below): A706P, T732I, G744T, A766I, A766L, G769K, T827K, T827N, I844E, I844Q, R847A, N856L, A899Q, G908A, G908N, T912I, T912L, L916F, Y917W, T941D, T94IN, A942D, A942P, N955D, A956I, A956L, A956V, K964E, D985N, E990R, T998Q, Q1005M, T1009I, L1012Q, 11013L, A1016I, A1020L, N1023K, S1051T, P1143N, and P1143Q.

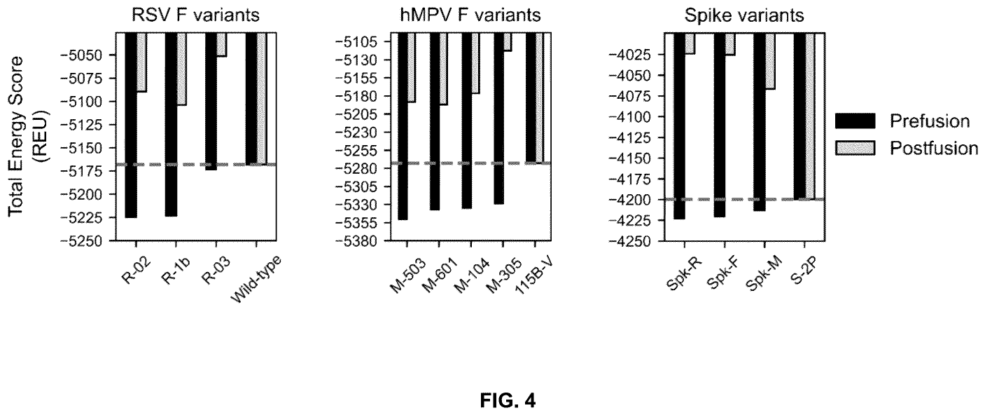

As the final objective was to find mutations working synergistically rather than individually, all substitutions favoring the prefusion state over the postfusion conformation were assessed in a combinatorial design step (FIG. 1). Though several sequences were found to lower the prefusion state while increasing the postfusion state energy, the number of mutations introduced was high (˜40 substitutions). Therefore, to prevent changes in the immunological properties of the proteins, the number of designable spots was decreased. We aimed to introduce less than 10 mutations by focusing on the lowest energy interactions and the preservation of newly introduced hydrogen bonds and salt bridges in the prefusion state, or substitutions with a strong negative effect on the postfusion structure's energy. The mutations of exemplary combinatorial variants for each protein are shown in Tables 5-7. The combinatorial design of these substitutions resulted in energetics gaps of at least 119 REU (FIG. 4 and Table 8).

TABLE 5
Mutations of exemplary combinatorial RSV F variants.*

| Position | R-1b | R-02 | R-03 |
|---|---|---|---|
| 55 (55) | S55A (S55A) | S55V (S55V)) | S55L (S55L) |
| 60 (60) | E60F (E60F) | E60F (E60F) | E60F (E60F) |
| 128 (150) | S128E (S150E) | S128L (S150L) | S128L (S150L) |
| 153 (175) | N153R (N175R) | N153R (N175R) | N153R (N175R) |
| 205 (227) | N205L (N227L) | N205L (N227L) | N205F (N227F) |
| 358 (380) | N358K (N380K) | N358K (N380K) | N358K (N380K) |
| 465 (487) | E465N (E487N) | E465V (E487V) | E465V (E487V) |

*Positions in parenthesis refer to the mutation position in the full-length constructs R-Base-FL (SEQ ID NO: 5) and R-1b-FL (SEQ ID NO: 6).

TABLE 6
Mutations of exemplary combinatorial hMPV F variants.

| Position | M-102 | M-104 | M-302 | M-305 | M-374 | M-404 | M-503 | M-601 |
|---|---|---|---|---|---|---|---|---|
| 42 | G42D | | | | | | | |
| 90 | A90N | A90N | A90N | A90N | | A90N | | |
| 106 | G106W | | G106W | G106R | | G106F | G106W | |
| 107 | A107F | | A107F | | | A107F | A107F | |
| 114 | | T114E | | | | | T114E | T114E |
| 116 | A116V | | A116V | | | A116V | | |
| 130 | L130D | L130D | | L130D | L130D | L130D | L130D | L130D |
| 149 | | | | | | | | S149T |
| 159 | A159L | A159L | A159L | A159I | | A159I | A159L | A159L |
| 162 | | | | | | | V162I | |
| 191 | | | | | | | | V191I |
| 203 | | V203I | | | | | V203I | |
| 209 | D209E | | D209E | | | D209E | | D209E |
| 216 | | | | | | | | A216R |
| 237 | | | | | S237H | | | |
| 277 | G277K | | G277E | G277D | | G277K | | |
| 314 | | | A314K | A314K | | A314K | | |
| 371 | | | | | S371P | | | |
| 393 | | | | | G393S | | | |
| 430 | | V430Q | | | | | V430Q | V430Q |
| 449 | V449D | V449D | | V449D | V449D | V449D | V449D | V449D |
| 453 | E453P | | | E453P | E453P | E453P | | |

TABLE 7
Mutations of exemplary combinatorial SARS-COV-2 S variants.

| Position | Spk-M | Spk-F | Spk-R | Spk-A* |
|---|---|---|---|---|
| 744 | | | G744T | |
| 769 | | G769K | G769K | G769K |
| 856 | N856L | | | |
| 899 | A899Q | | | |
| 916 | L916F | | | L916F |
| 917 | Y917W | | | |
| 941 | T941D | T941D | T941D | T941N |
| 942 | | | | A942P |
| 955 | | | N955D | |
| 956 | A956L | A956L | | |
| 964 | K964E | | | |
| 985 | D985N | | D985N | |
| 990 | | E990R | E990R | |
| 1005 | | | | Q1005M |
| 1009 | | | | T1009I |
| 1012 | | | | L1012Q |
| 1013 | | | | I1013L |
| 1016 | | A1016I | A1016I | A1016I |
| 1020 | | | | A1020L |
| 1023 | | | | N1023K |
| 1051 | | | | S1051T |
| 1143 | P1143Q | P1143Q | P1143N | |

*Spk-A was designed with a modified protocol.

TABLE 8
Energetic differences between pre- and postfusion states, stabilizing mutations, and thermal stability of designed fusion proteins.

| | | Energy | Stabilizing mutations | | | | | | |
|---|---|---|---|---|---|---|---|---|---|
| | | gap | | Inter- | Intra- | | | | |
| | | (REU) | | protomer | protomer | Reduction of | Decrease | | Melting |
| | Protein | Pre -vs- | Cavity | polar | polar | unsatisfied | charge | Postfusion | temperature |
| Virus | variants | Postfusion | filling | interactions | interactions | polars | repulsion | destabilizing | (° C.) |
| RSV | Base | 0* | N/A | N/A | N/A | N/A | N/A | N/A | N/A |
| | construct | | | | | | | | |
| | (R-Base) | | | | | | | | |
| | R-1b | 119.1 | E60F | S128E^{#} | N358K^{#} | S55A | E465N^{#} | N153R^{#} | 62 |
| | | | | | | N205L^{#} | | | |
| | R-02 | 135.1 | E60F | N/A | N358K^{#} | S55V | E465V^{#} | N153R^{#} | N/A |
| | | | S128L^{#} | | | N205L^{#} | | | |
| | R-03 | 121.7 | E60F | N/A | N358K^{#} | S55L | E465V^{#} | N153R^{#} | N/A |
| | | | S128L^{#} | | | N205F^{#} | | | |
| hMPV | Base | 0* | N/A | N/A | N/A | N/A | N/A | N/A | 54.8 |
| | construct | | | | | | | | |
| | (115B-V) | | | | | | | | |
| | (M-Base) | | | | | | | | |
| | M-104 | 158.2 | A159L | A90N | V430Q | N/A | N/A | L130D | 61.5 |
| | | | V203I | T114E | V449D | | | | |
| | M-305 | 210.7 | A159I | A90N | G106R | N/A | E453P | L130D | 56.7 |
| | | | | | G277D | | | | |
| | | | | | A314K | | | | |
| | | | | | V449D | | | | |
| | M-503 | 161.8 | G106W | T114E | V430Q | N/A | N/A | L130D | N/A |
| | | | A107F | | V449D | | | | |
| | | | A159L | | | | | | |
| | | | V162I | | | | | | |
| | | | V203I | | | | | | |
| | M-601 | 144.4 | A159L | T114E | V430Q | S149T | N/A | L130D | N/A |
| | | | V191I | D209E | V449D | | | | |
| | | | | A216R | | | | | |
| SARS-CoV-2 | Base | 0* | N/A | N/A | N/A | N/A | N/A | N/A | 45.7^{a} |
| | construct | | | | | | | | 62.5^{b} |
| | (S-2P) | | | | | | | | |
| | (Spk-Base) | | | | | | | | |
| | Spk-M | 146.1 | L916F | A899Q | K964E | N856L | D985N | T941D | 61 |
| | | | Y917W | | P1143Q | | | | |
| | | | A956L | | | | | | |
| | Spk-F | 194.9 | A956L | E990R | G769K | N/A | N/A | T941D | 61.5 |
| | | | A1016I | | P1143Q | | | | |
| | Spk-R | 198.4 | A1016I | E990R | G744T | N/A | D985N | T941D | 46.5^{a} |
| | | | | | G769K | | | | 60.3b |
| | | | | | N955D | | | | |
| | | | | | P1143N | | | | |

*The base construct's pre- and postfusion energies are normalized.
N/A = not applicable
^{a}First apparent melting temperature
^{b}Second apparent melting temperature
^{#}Positions S128, N153, N205, N358, and E465 in R-1b, R-02, and R-03 correspond with positions S150, N175, N227, N380, and E487, respectively, in the native RSV F protein.

Biochemical Characterization of RSV F, hMPV F and SARS-COV-2 S Variants

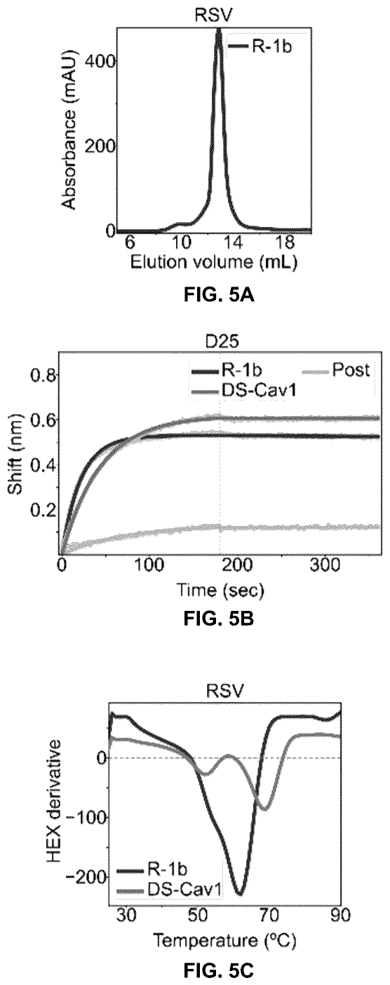

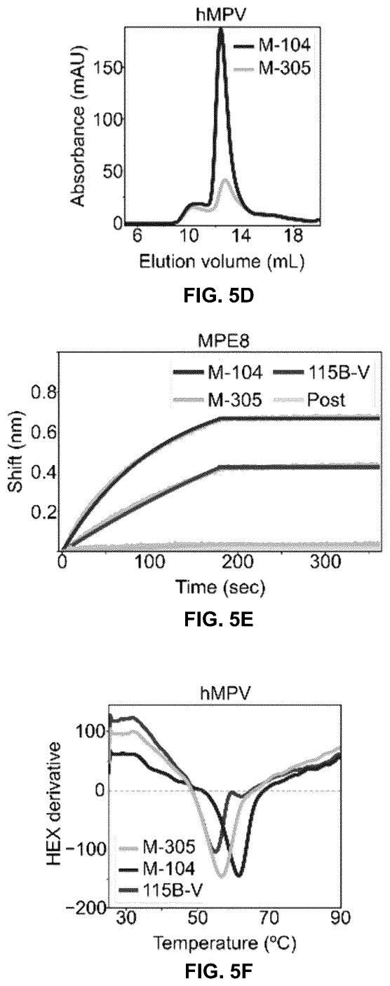

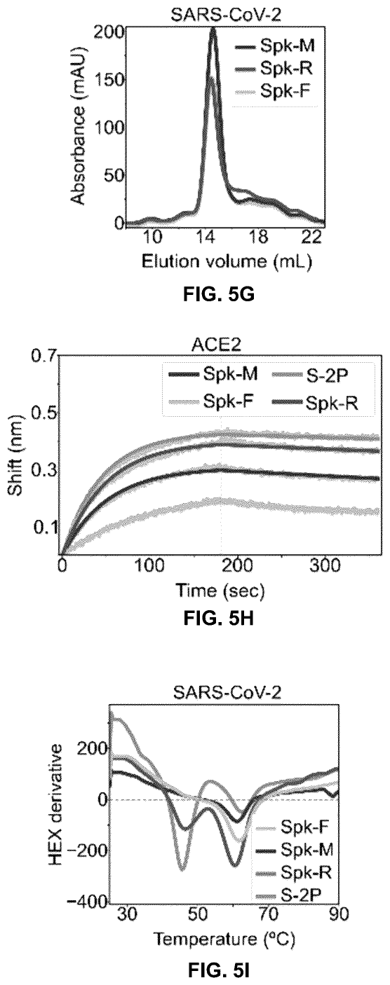

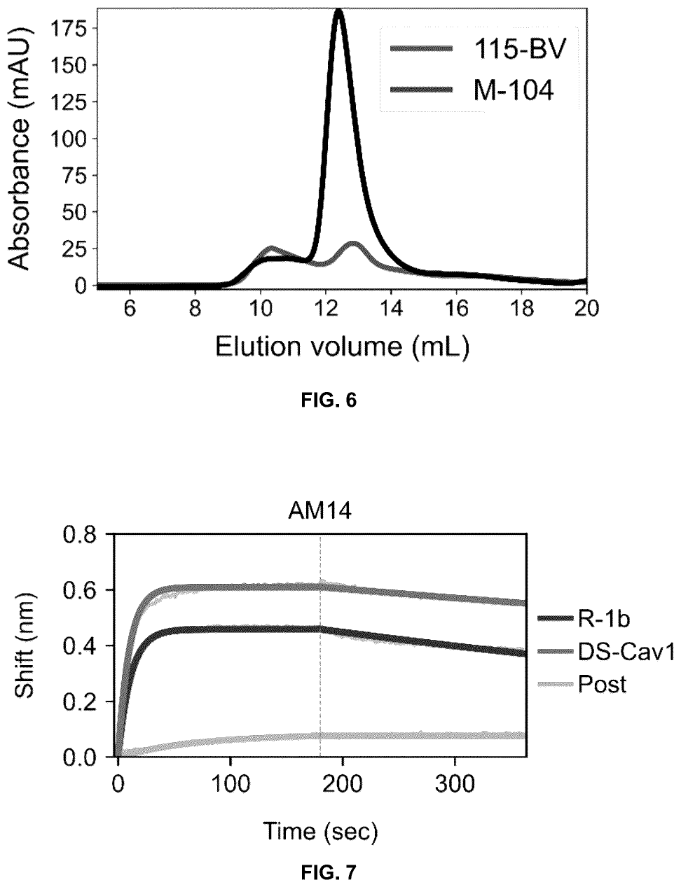

After ranking designed sequences based on their energy gap between states, the top 3-4 variants were expressed and purified. For RSV F, one (R-1b) out of three designs (R-1b, R-02, and R-03) was found to be a monodispersed and trimeric protein, as evaluated by size exclusion chromatography (SEC) (FIG. 5A). For the hMPV F and SARS-COV-2 S, two (M-104 and M-305) out of four (M-104, M-305, M-503, and M-601), and three (Spk-M, Spk-F, and Spk-R) out of three redesigned proteins behaved similarly, respectively (FIGS. 5D and 5G). Spk-A, which was designed using different methods, was also expressed, purified, and found to be a monodispersed and trimeric protein. Remarkably, the hMPV F variant M-104 showed high expression yields, with a 5.5-fold increase with respect to its parent construct the semi-stabilized hMPV F 115-BV²⁷ (FIG. 6). The structural state of these expressed constructs was then evaluated based on their conservation of prefusion-specific epitopes. As predicted, all designs presented a prefusion-like structure as they tightly bound to prefusion-specific binders such as the RSV F antibodies D25^{51,52} and AM14^{52,53} (FIG. 5B and FIG. 7), the hMPV F antibodies MPE8⁵⁴ or 465⁵⁵ (FIG. 5E and FIG. 8), or the angiotensin-converting enzyme 2 (ACE2)⁵⁰ for the SARS-COV-2 S protein (FIG. 5H). Binding constants are shown in Tables 9, 10, and 11.

TABLE 9
Binding kinetics of RSV F variants obtained by biolayer interferometry.

| | Assay | Antibodies | | | | | | | | |
|---|---|---|---|---|---|---|---|---|---|---|
| Protein | temperature | D25 | | | AM14 | | | 131-2a | | |
| Variants | (° C.) | koff(1/s) | kon(1/Ms) | KD(M) | koff(1/s) | kon(1/Ms) | KD(M) | koff(1/s) | kon(1/Ms) | KD(M) |
| R-1b | RT | N/A | 1.79E+05 | N/A | 1.24E−04 | 2.19E+05 | 5.68E−10 | 4.00E−04 | 3.37E+04 | 1.19E−08 |
| | | | 1.51E+05 | | 1.01E−04 | 2.03E+05 | 4.97E−10 | 3.10E−04 | 4.79E+04 | 6.47E−09 |
| | | | 1.44E+05 | | 1.11E−04 | 2.27E+05 | 4.89E−10 | 3.53E−04 | 4.75E+04 | 7.43E−09 |
| | 55 | N/A | 1.56E+05 | N/A | N/A | 8.87E+04 | N/A | N/A | N/A | N/A |
| | | | 1.20E+05 | | 1.15E−05 | 8.63E+04 | 1.34E−10 | | | |
| | | | 1.21E+05 | | 1.31E−05 | 9.91E+04 | 1.33E−10 | | | |
| | 60 | N/A | 92.7 | N/A | N.B | N.B | N.B | N.B | N.B | N.B |
| | | | 83.8 | | | | | | | |
| | | | 54.6 | | | | | | | |
| DS-Cav1 | RT | N/A | 1.21E+05 | N/A | N/A | 1.61E+05 | NA | 0.001 | 8.29E+03 | 1.33E−07 |
| | | | 1.87E+05 | | | 2.04E+05 | | 3.17E−04 | 9.19E+03 | 3.44E−08 |
| | | | 1.49E+05 | | | 1.81E+05 | | 3.07E−04 | 1.76E+04 | 1.74E−08 |
| | 55 | N/A | 2.13E+05 | N/A | N/A | 5.03E+04 | NA | N/A | N/A | N/A |
| | | | 1.60E+05 | | | 5.85E+04 | | | | |
| | | | 1.42E+05 | | | 5.01E+04 | | | | |
| | 60 | N/A | 1.15E+05 | N/A | N/A | 3.02E+04 | NA | N/A | N/A | N/A |
| | | | 1.35E+05 | | | 1.31E+04 | | | | |
| | | | 1.32E+05 | | | 5.39E+03 | | | | |
| RSVF A2 | RT | N.B | N.B | N.B | N.B | N.B | N.B | N/A | 4.00E+05 | N/A |
| (postfusion) | | | | | | | | | 4.09E+05 | |
| | | | | | | | | | 4.85E+05 | |
| | 60 | N/A | N/A | N/A | N/A | N/A | N/A | N/A | 5.22E+05 | N/A |
| | | | | | | | | | 4.82E+05 | |
| | | | | | | | | | 4.52E+05 | |

RT = room temperature
N.B = no binding
N/A = not applicable

TABLE 10
Binding kinetics of hMPV F variants obtained by biolayer interferometry.

| | Assay | Antibodies | | | | | |
|---|---|---|---|---|---|---|---|
| Protein | temperature | MPE8 | | | 465 | | |
| Variants | (° C.) | koff(1/s) | kon(1/Ms) | KD(M) | koff(1/s) | kon(1/Ms) | KD(M) |
| M-104 | RT | N/A | 4.01E+04 | NA | N/A | 1.94E+05 | N/A |
| | | | 3.24E+04 | | | 2.00E+05 | |
| | | | 4.21E+04 | | | 1.93E+05 | |
| | 55 | N/A | 3.66E+04 | N/A | 6.60E−05 | 7.32E+04 | 9.02E−10 |
| | | | 2.17E+04 | | 6.85E−05 | 7.21E+04 | 9.51E−10 |
| | | | 2.51E+04 | | 4.25E−05 | 7.18E+04 | 5.92E−10 |
| | 60 | N.B | N.B | N.B | 2.29E−04 | 2.29E+04 | 1.00E−08 |
| | | | | | N/A | 2.29E+04 | N/A |
| | | | | | 2.62E−04 | 2.56E+04 | 1.02E−08 |
| M-305 | RT | N.B | N.B | N.B | 4.91E−03 | 1.08E+05 | 4.52E−08 |
| | | | | | 4.31E−03 | 1.19E+05 | 3.61E−08 |
| | | | | | 4.86E−03 | 1.08E+05 | 4.51E−08 |
| 115B-V | RT | N/A | 1.45E+04 | NA | N/A | 7.99E+04 | N/A |
| | | | 1.52E+04 | | | 7.91E+04 | |
| | | | 1.09E+04 | | | 7.79E+04 | |
| | 55 | N.B | N.B | N.B | N.B | N.B | N.B |
| Postfusion | RT | N.B | N.B | N.B | N.B | N.B | N.B |

RT = room temperature
N.B = no binding
N/A = not applicable

TABLE 11
Binding kinetics of SARS-COV-2 S variants obtained
by biolayer interferometry.

| | Assay | Prefusion binder | | |
|---|---|---|---|---|
| Protein | temperature | ACE2 | | |
| Variants | (° C.) | koff (1/s) | kon (1/Ms) | KD (M) |
| Spk-M | RT | 5.69E−04 | 4.73E+04 | 1.20E−08 |
| | | 5.54E−04 | 4.89E+04 | 1.13E−08 |
| | | 5.82E−04 | 4.71E+04 | 1.24E−08 |
| | 55 | 7.12E−04 | 2.92E+04 | 2.44E−08 |
| | | 6.06E−04 | 2.75E+04 | 2.21E−08 |
| | | 5.41E−04 | 2.72E+04 | 1.99E−08 |
| Spk-F | RT | 1.07E−03 | 2.43E+04 | 4.42E−08 |
| | | 7.14E−04 | 2.32E+04 | 3.08E−08 |
| | | 7.86E−04 | 2.61E+04 | 3.01E−08 |
| Spk-R | RT | 4.00E−04 | 4.58E+04 | 8.73E−09 |
| | | 4.61E−04 | 4.61E+04 | 1.00E−08 |
| | | 3.43E−04 | 5.03E+04 | 6.81E−09 |
| S-2P | RT | N/A | 4.26E+04 | N/A |
| | | 2.04E−04 | 5.60E+04 | 3.64E−09 |
| | | N/A | 3.93E+04 | NA |
| | 55 | 4.70E−04 | 2.30E+04 | 2.04E−08 |
| | | 3.08E−04 | 1.23E+04 | 2.51E−08 |
| | | 1.93E−03 | 7.03E+03 | 2.74E−07 |

RT = room temperature
N/A = not applicable

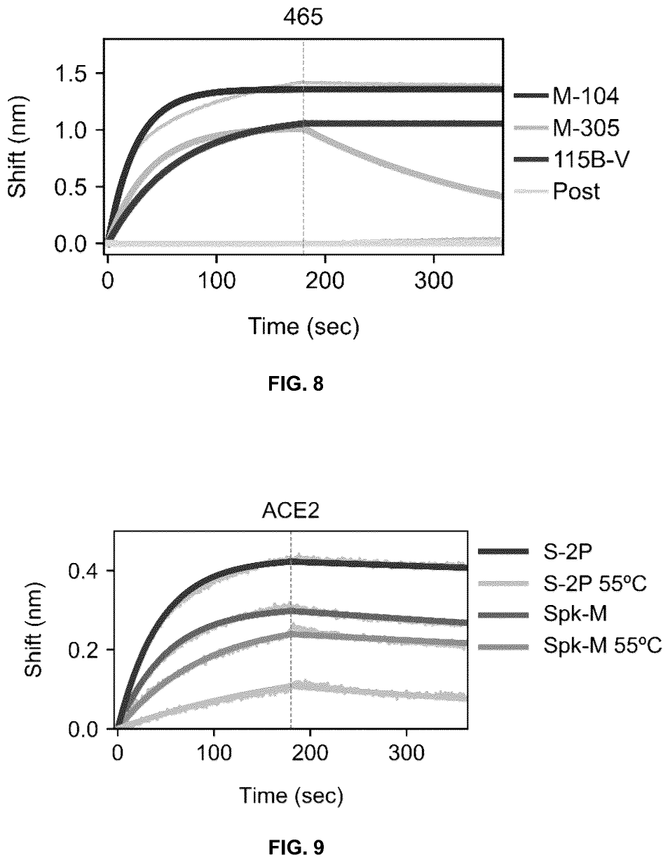

All expressed proteins showed improved thermal stability compared to their parent prefusion constructs (Table 8). The spike variants Spk-M and Spk-F displayed the highest melting temperature improvement, with ˜15° C. increment above the current SARS-COV-2 vaccine, the S-2P construct⁶ (FIG. 5I). Unlike the vaccine S-2P⁴⁶, the design Spk-M preserved the prefusion conformation even after one hour heating at 55° C. as evidenced by ACE2 binding at this temperature (FIG. 9, Table 11). This stability seems to compare with the highly stable HexaPro construct, which was achieved by introducing several proline substitutions and experimentally evaluating 100 variations²⁵.

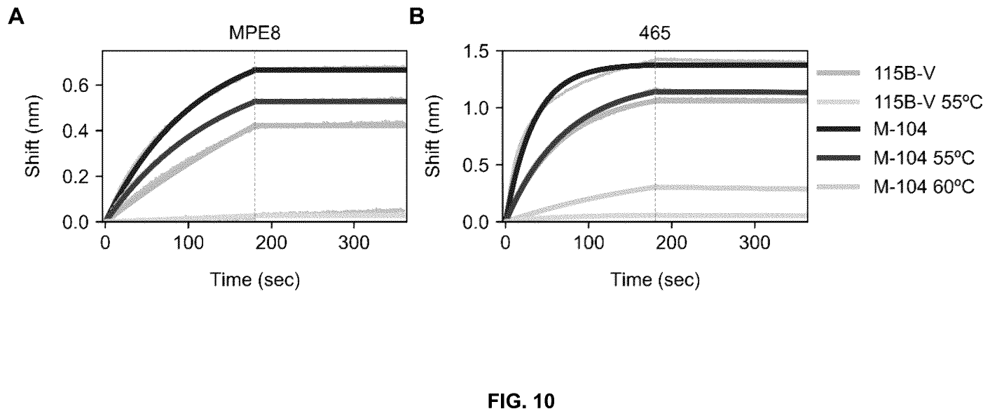

A similar scenario was observed in the hMPV F variant M-104 whose stability could compare to variants containing additional disulfide bonds⁶². M-104 shows a higher melting point (61.5° C., FIG. 5F) than the hMPV F variant DS-CavEs (60.7° C.)⁶² which has two designed disulfide bonds. Additionally, M-104 remains antigenically unaltered after heating at 55° C. (FIG. 10), as has been seen in DS-CavEs2⁶² which contains four new disulfide bonds. Although testing under identical conditions is required to validate these comparisons, these results demonstrate how the correct placement of electrostatic interactions can lead to highly stable proteins.

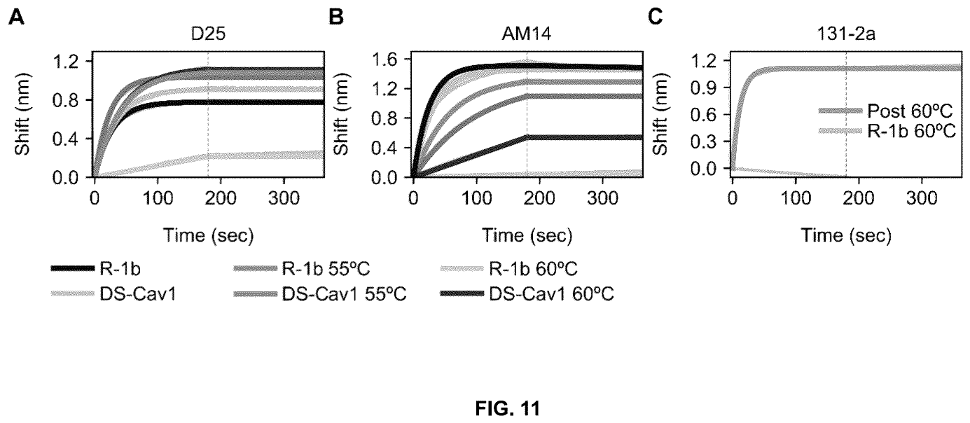

For RSV F, the improvement of the prefusion state stability cannot be well estimated as we started with the wild-type sequence³⁰ whose instability substantially impedes its production as an isolated soluble prefusion-state protein²¹; all purified RSV F molecules are found mostly in its postfusion state³¹. Therefore, obtaining the R-1b variant with a melting temperature of 62° C. revealed an effective optimization of the sequence to stay in the prefusion conformation (FIG. 5C); especially since no disulfide bonds were introduced and stabilization was achieved through the optimization of non-covalent interactions. Remarkably, design R-1b proved to be antigenically intact even after heating at 55° C. (FIG. 11 (A,B)).

The Spk-A protein was designed with a modified protocol. We predict Spk-A will have similar properties to Spk-M, Spk-F, and Spk-R.

Structure Determination of Leading RSV F, hMPV F and SARS-COV-2 S Variants

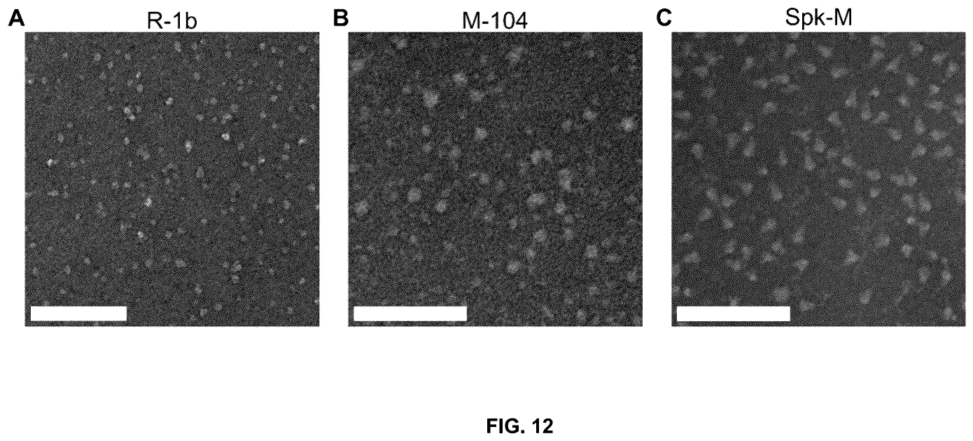

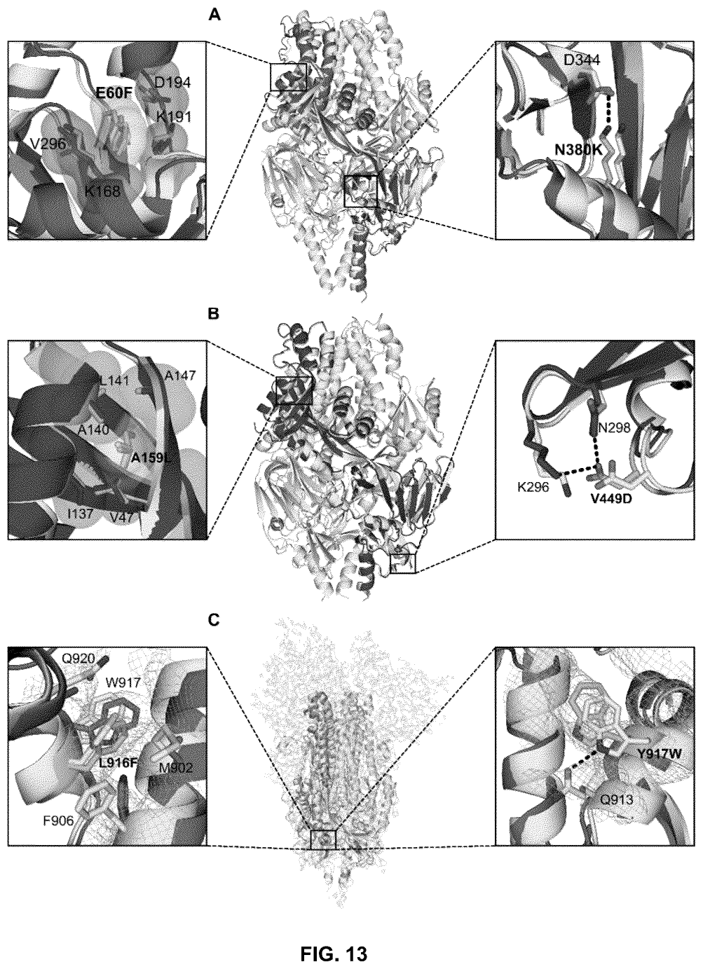

Negative-stain electron microscopy (EM) confirmed homogeneous trimeric prefusion morphology of leading candidates for all the three different fusion proteins studied (FIG. 12). We therefore proceeded to obtain atomic details by x-ray crystallization and cryo-EM. The crystal structure of the variants R-1b and M-104 confirmed their prefusion conformation at a resolution of 3.1 Å and 2.4 Å, respectively (FIG. 13 (A,B)). The accuracy of our computational predictions was reflected on the high structural similarity between the determined structures and the computational models, with root-mean-square deviations (RMSDs) of only 1.193 Å (405 Cαatoms) for R-1b, and 0.53 Å (416 Cαatoms) for M-104. The 3D classification performed on the spike cryo-EM images also verified the prefusion structure of the protein with all particles displaying one receptor binding domain (RBD) in the up conformation. Solving the structure at 3.7 Å resolution, revealed that the S2 subunit, which was the only part engineered, agreed closely with the computational model with a RMSD of only 1.345 Å (377 Cαatoms) (FIG. 13(C)).

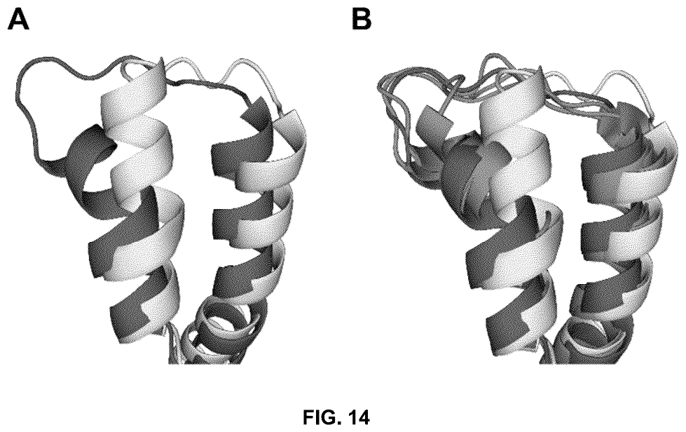

Although no significant perturbations were observed in all our variants overall, subtle differences at the antigenic site Ø were identified between R-1b and its parent RSV F protein. Specifically, the α4 helix in R-1b is bent towards residue D200 (FIG. 14(A)), when compared to parent RSV F protein. As the antigenic site Ø is intrinsically flexible, structural variations in this region are expected and have been observed in several prefusion-stabilized RSV F proteins, including the clinical candidate DS-Cav1^{13,21,22} (FIG. 14(B)). For future stabilization efforts, this could be another area of interest for stabilization.

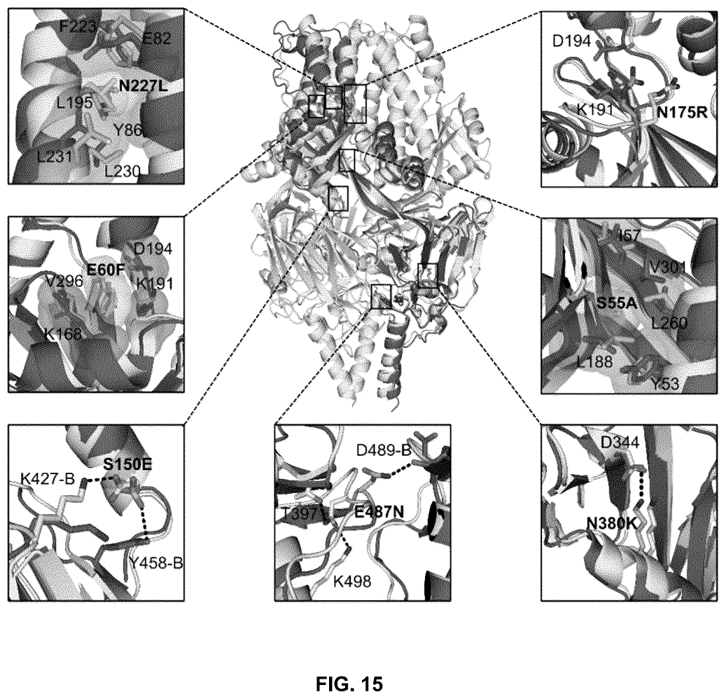

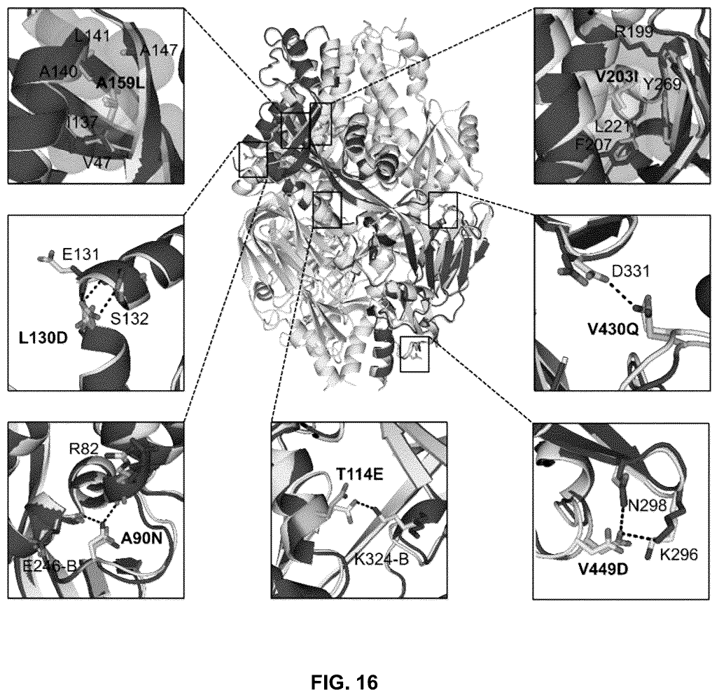

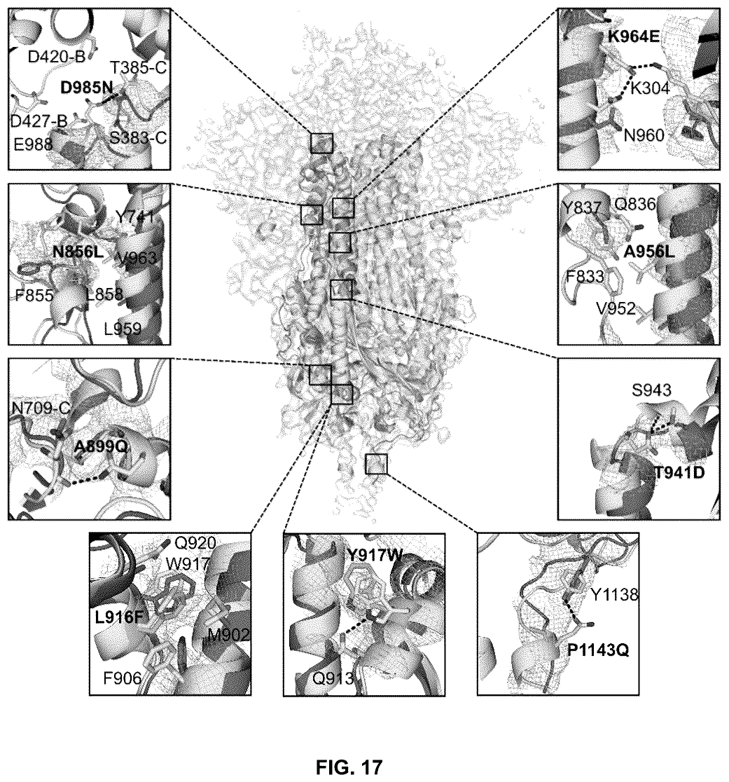

The crystal structures of R-1b and M-104 revealed that despite some deviations between designed and crystal structure, most introduced substitutions followed their predicted stabilization mechanism by either filing cavities or increasing intra- or interprotomer hydrogen bonds and salt bridges (FIGS. 15 and 16 and Table 8). Especially precise rotamer agreement between our computational models and the experimental data was found in cavity filling mutations, mainly the E60F in R-1b, and the A159L and V203I in M-104 (FIG. 13 (A,B) and FIG. 16). Significant rotamer agreement was also observed in substitutions increasing polar interactions, such as the N358K in R-1b (corresponding to a N380K mutation in the native RSV F protein), and the L130D, V430Q, and V449D in M-104 (FIG. 13 (A,B) and FIG. 16). Other mutations such as S128E and E465N in R-1b (corresponding to S150E and E487N mutations, respectively, in the native RSV F protein) did not interact with the predicted residues but still contributed to the prefusion stability by increasing polar interactions at the protomers' interface (FIG. 15). Finally, the design Spk-M was stabilized by four substitutions filling cavities and five substitutions increasing polar interactions at the S2 subunit, three of which were interprotomer contacts (FIG. 13(C), FIG. 17, and Table 8).

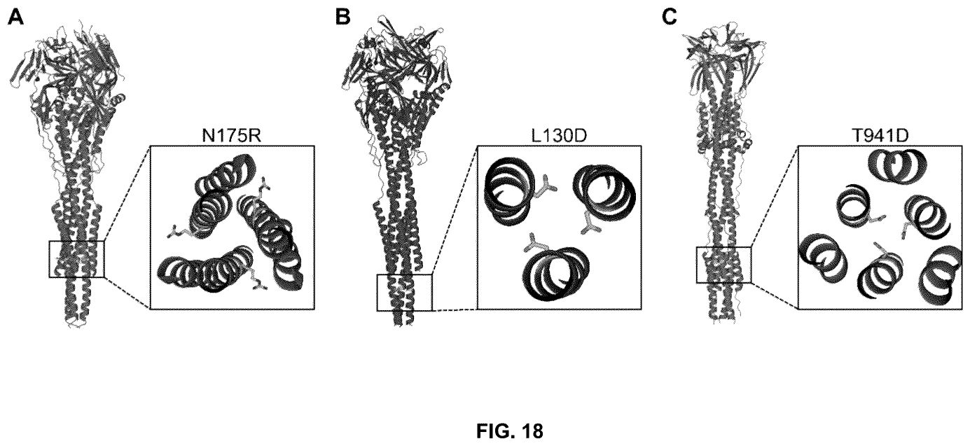

Although most of the designed substitutions stabilized the prefusion state, the mutations N153R in R-1b (corresponding to a N175R mutation in the native RSV F protein), L130D in M-104, and T941D in Spk-M were intended to disrupt the postfusion conformation (FIG. 18). These residues are solvent-accessible in the prefusion state and therefore should not have an impact on the stability of that conformation. However, the postfusion conformation places these residues at the six-helix bundle where unsatisfied polar residues are highly unfavored and thereby likely to disrupt the core. As we had the postfusion-specific antibody 131-2A⁵⁶, we sought to prove this hypothesis and confirm that we had not only stabilized the prefusion state but also indeed destabilized the postfusion state. The diminished binding of the 131-2A antibody to R-1b after heating (60° C.) (FIG. 11(C)), which should have converted the protein into its postfusion state, proved that we fulfilled the design objectives we set out for.

### Immunogenicity of Design R-1b

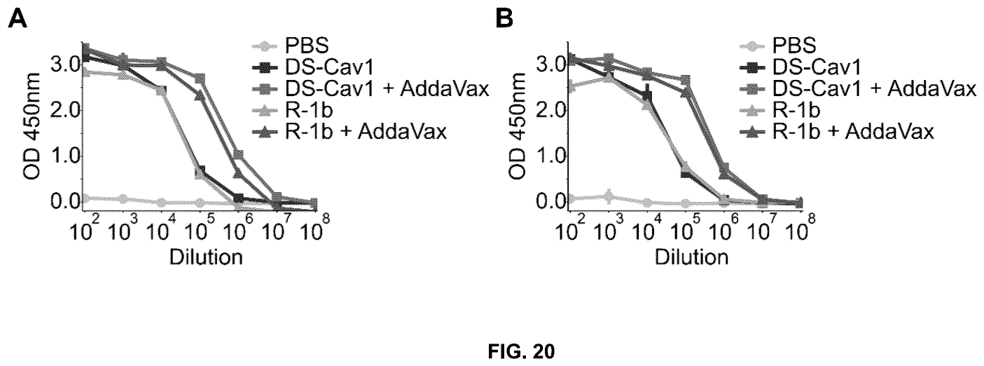

We selected the R-1b variant for a vaccination study due to the availability of a highly stable prefusion control, such as the clinical candidate DS-Cav1¹³. Therefore, to investigate the effect of the introduced mutations on the RSV F immunogenicity, female BALB/c mice were vaccinated twice with either 0.2 or 2 μg of purified R-1b or DS-Cav1 with or without AddaVax adjuvant. Mice were bled at three and nine weeks post-second immunization (FIG. 19(A)). Sera analysis for binding to prefusion RSV F and RSV A2 neutralization revealed that R-1b induced similar levels of RSV F-specific antibody titers (FIG. 19 (B,C) and FIG. 20) and comparable neutralizing activity related to DS-Cav1 (FIG. 19(D)).

### Disulfide Bonds for Enhanced Prefusion Stability

We predicted that introducing cysteines into the constructs at various positions would confer enhanced prefusion stability through disulfide bonding.

For R-1b and the other RSV F constructs, these positions include positions 52 and 128 (positions 52 and 150 in the full-length constructs), positions 55 and 166 (positions 55 and 188 in the full-length constructs), positions 60 and 174 (positions 60 and 196 of the full-length constructs), positions 135 and 161 (positions 157 and 183 of the full-length constructs), positions 155 and 268 (positions 155 and 290 of the full-length constructs), and positions 421 and 444 (positions 443 and 466 of the full length constructs). Positions 55 and 166, positions 135 and 161, and positions 421 and 444 have been tested and confirmed to enhance prefusion stability in constructs containing the R-1b mutations (except at position 55, in which a cysteine has replaced the alanine in R-1b).

For the hMPV constructs, these positions include positions 43 and 120, positions 45 and 157, positions 113 and 336, positions 115 and 375, positions 123 and 429, and positions 211 and 253. Positions 115 and 375, positions 123 and 429, and positions 211 and 253 have been tested and confirmed to enhance prefusion stability in the M-104 construct.

### General Impact

Detailed antibody response studies have illustrated that prefusion-stabilized class I fusion proteins are potent immunogens and promising vaccine candidates. This has been proven to be true for several viruses, including RSV^{13,21,22}, hMPV⁶², parainfluenza⁸, Nipah¹¹, MERS-Cov²⁰, and SARS-COV-2²⁵. Several of these immunogens have been developed by many steps of iterations of manual structure-based design with experimental evaluation, often testing hundreds of combinations of mutations^{8,11,21-23,25-27}. To alleviate this laborious exploratory testing, we automated one of the underlying principles behind their stabilization efforts that considers the biophysics of the fusion protein and its large irreversible switch. We developed a computational approach that seeks to freeze the prefusion conformation by learning about suboptimal contacts from its alternate conformation. Our algorithm allows the automated identification of these regions, and their potential substitutions, based on energy differences and relative motion between the two states. We acknowledge that our computational approach can be limited by the need for both pre- and postfusion structures. However, we believe that the high accuracy of current protein structure prediction algorithms could alleviate this drawback⁶³. The efficiency of our method has been demonstrated on three different fusion proteins, such as the RSV F, hMPV F, and the SARS-COV-2 S, where only 3-4 variants were necessary to successfully find a stable prefusion design. Additionally, we were able to validate the immunogenicity of one design in a mouse model, showing similar in vitro neutralization and specific serum IgG patterns compared to a clinical candidate. Therefore, our algorithm could highly impact the vaccine development field by allowing a rapid optimization of both novel class I fusion proteins as well as leading vaccine immunogens.

### REFERENCES

1. Doms, R. W. & Moore, J. P. HIV-1 membrane fusion: Targets of opportunity. Journal of Cell Biology vol. 151 https://www.ncbi.nlm.nih.gov/pmc/articles/PMC2192632/(2000).
2. Moller-Tank, S. & Maury, W. Ebola Virus Entry: A Curious and Complex Series of Events. PLOS Pathog. 11, e1004731 (2015).
3. Chang, A. & Dutch, R. E. Paramyxovirus fusion and entry: multiple paths to a common end. Viruses 4, 613-36 (2012).
4. Pabis, A., Rawle, R. J. & Kasson, P. M. Influenza hemagglutinin drives viral entry via two sequential intramembrane mechanisms. PNAS (2020) doi:10.1101/2020.01.30.926816.
5. Shang, J. et al. Cell entry mechanisms of SARS-COV-2. Proc. Natl. Acad. Sci. U.S.A 117, 11727-11734 (2020).
6. Harrison, S. C. Viral membrane fusion. Virology 479-480, 498-507 (2015).
7. Rey, F. A. & Lok, S. M. Common Features of Enveloped Viruses and Implications for Immunogen Design for Next-Generation Vaccines. Cell vol. 172 1319-1334 (2018).
8. Stewart-Jones, G. B. E. E. et al. Structure-based design of a quadrivalent fusion glycoprotein vaccine for human parainfluenza virus types 1-4. Proc. Natl. Acad. Sci. U.S.A. 115, 12265-12270 (2018).
9. Dang, H. V. et al. An antibody against the F glycoprotein inhibits Nipah and Hendra virus infections. Nat. Struct. Mol. Biol. 26, 980-987 (2019).
10. Ngwuta, J. O. et al. Prefusion F-specific antibodies determine the magnitude of RSV neutralizing activity in human sera. Sci. Transl. Med. 7, 309ra162 (2015).
11. Loomis, R. J. et al. Structure-Based Design of Nipah Virus Vaccines: A Generalizable Approach to Paramyxovirus Immunogen Development. Front. Immunol. 11, 1-16 (2020).
12. Falloon, J. et al. An Adjuvanted, Postfusion F Protein-Based Vaccine Did Not Prevent Respiratory Syncytial Virus Illness in Older Adults. J. Infect. Dis. 216, 1362-1370 (2017).
13. Mclellan, J. S. et al. Structure-Based Design of a Fusion Glycoprotein Vaccine for Respiratory Syncytial Virus. Science 342, 592 (2013).
14. Baden, L. R. et al. Efficacy and Safety of the mRNA-1273 SARS-COV-2 Vaccine. N. Engl. J. Med. 384, 403-416 (2021).
15. Harbury, P. B., Zhang, T., Kim, P. S. & Alber, T. A Switch Between Two-, Three-, and Four-stranded Coiled Coils in GCN4 Leucine Zipper Mutants. Science (80-.). 262, 1401-1407 (1993).
16. Miroshnikov, K. A. et al. Engineering trimeric fibrous proteins based on bacteriophage T4 adhesins. Protein Eng. 11, 329-332 (1998).
17. Chen, B. et al. A Chimeric Protein of Simian Immunodeficiency Virus Envelope Glycoprotein gp140 and Escherichia coli Aspartate Transcarbamoylase. J. Virol. 78, 4508-4516 (2004).
18. Wang, J. Y. et al. Improved expression of secretory and trimeric proteins in mammalian cells via the introduction of a new trimer motif and a mutant of the tPA signal sequence. Appl. Microbiol. Biotechnol. 91, 731-740 (2011).
19. Narkhede, Y. B., Gonzalez, K. J. & Strauch, E. M. Targeting Viral Surface Proteins through Structure-Based Design. Viruses 2021, Vol. 13, Page 1320 13, 1320 (2021).
20. Pallesen, J. et al. Immunogenicity and structures of a rationally designed prefusion MERS-COV spike antigen. Proc. Natl. Acad. Sci. U.S.A 114, E7348-E7357 (2017).
21. Krarup, A. et al. A highly stable prefusion RSV F vaccine derived from structural analysis of the fusion mechanism. Nat. Commun. 6, 8143 (2015).
22. Joyce, M. G. et al. Iterative structure-based improvement of a fusion-glycoprotein vaccine against RSV. Nat. Struct. Mol. Biol. 23, 811-820 (2016).
23. Lucy Rutten, Morgan S. A. Gilman, Sven Blokland, Jarek Juraszek, Jason S. Mclellan, J. P. M. L. Structure-Based Design of Prefusion-Stabilized Filovirus Glycoprotein Trimers. 19-21 (2020).
24. Rutten, L. et al. Structure-Based Design of Prefusion-Stabilized Filovirus Glycoprotein Trimers. Cell Rep. 30, 4540 (2020).
25. Hsieh, C. L. et al. Structure-based design of prefusion-stabilized SARS-COV-2 Spikes. Science (80-.). 369, 1501-1505 (2020).
26. Wong, J. J. W., Paterson, R. G., Lamb, R. A. & Jardetzky, T. S. Structure and stabilization of the Hendra virus F glycoprotein in its prefusion form. Proc. Natl. Acad. Sci. U.S.A 113, 1056-1061 (2016).
27. Battles, M. B. et al. Structure and immunogenicity of prefusion-stabilized human metapneumovirus F glycoprotein. Nat. Commun. 8, 1-11 (2017).
28. Davey, J. A. & Chica, R. A. Multistate approaches in computational protein design. Protein Sci. 21, 1241-1252 (2012).
29. Leaver-Fay, A., Jacak, R., Stranges, P. B. & Kuhlman, B. A generic program for multistate protein design. PLOS One 6, (2011).
30. Tian, D. et al. Structural basis of respiratory syncytial virus subtype-dependent neutralization by an antibody targeting the fusion glycoprotein. Nat. Commun. 8, 1-7 (2017).
31. McLellan, J. S., Yang, Y., Graham, B. S. & Kwong, P. D. Structure of Respiratory Syncytial Virus Fusion Glycoprotein in the Postfusion Conformation Reveals Preservation of Neutralizing Epitopes. J. Virol. 85, 7788-7796 (2011).
32. Wang, R. Y. R. et al. Automated structure refinement of macromolecular assemblies from cryo-EM maps using Rosetta. Elife 5, (2016).
33. Adams, P. D. et al. PHENIX: a comprehensive Python-based system for macromolecular structure solution. urn:issn: 0907-4449 66, 213-221 (2010).
34. Tyka, M. D. et al. Alternate states of proteins revealed by detailed energy landscape mapping. J. Mol. Biol. 405, 607-618 (2011).
35. Chen, V. B. et al. MolProbity: all-atom structure validation for macromolecular crystallography. Acta Crystallogr. Sect. D Biol. Crystallogr. 66, 12-21 (2010).
36. Más, V. et al. Engineering, Structure and Immunogenicity of the Human Metapneumovirus F Protein in the Postfusion Conformation. PLOS Pathog. 12, e1005859 (2016).
37. Walls, A. C. et al. Structure, Function, and Antigenicity of the SARS-COV-2 Spike Glycoprotein. Cell 181, 281-292.e6 (2020).
38. Cai, Y. et al. Distinct conformational states of SARS-COV-2 spike protein. Science (80-.). 369, (2020).
39. Song, Y. et al. High-Resolution Comparative Modeling with RosettaCM. Structure 21, 1735-1742 (2013). 40 Lan, J. et al. Structure of the SARS-COV-2 spike receptor-binding domain bound to the ACE2 receptor. Nat. 2020 5817807 581, 215-220 (2020).
41. Xia, S. et al. Inhibition of SARS-COV-2 (previously 2019-nCOV) infection by a highly potent pan-coronavirus fusion inhibitor targeting its spike protein that harbors a high capacity to mediate membrane fusion. Cell Res. 2020 304 30, 343-355 (2020).
42. Fleishman, S. J. et al. Rosettascripts: A scripting language interface to the Rosetta Macromolecular modeling suite. PLOS One 6, e20161 (2011).
43. Park, H. et al. Simultaneous Optimization of Biomolecular Energy Functions on Features from Small Molecules and Macromolecules. J. Chem. Theory Comput. 12, 6201-6212 (2016).
44. Maguire, J. B. et al. Perturbing the energy landscape for improved packing during computational protein design. Proteins Struct. Funct. Bioinforma. 89, 1-14 (2020).
45. Bhardwaj, G. et al. Accurate de novo design of hyperstable constrained peptides. Nat. 2016 5387625 538, 329-335 (2016). 46 Wrapp, D. et al. Cryo-EM structure of the 2019-nCOV spike in the prefusion conformation. Science (80-.). 367, 1260-1263 (2020).
47. Mclellan, J. S., Yang, Y., Graham, B. S. & Kwong, P. D. Structure of Respiratory Syncytial Virus Fusion Glycoprotein in the Postfusion Conformation Reveals Preservation of Neutralizing Epitopes †. J. Virol. 85, 7788-7796 (2011).
48. Huang, J. et al. Structure, Immunogenicity, and Conformation-Dependent Receptor Binding of the Postfusion Human Metapneumovirus F Protein STRUCTURE AND ASSEMBLY. J. Virol. jvi.asm.org 95, 593-614 (2021).
49. Fang, L. Codon Optimization. (2020). 50 Chan, K. K. et al. Engineering human ACE2 to optimize binding to the spike protein of SARS coronavirus 2. Science (80-.). 369, 1261-1265 (2020).
51. Mclellan, J. S. et al. Structure of RSV fusion glycoprotein trimer bound to a prefusion-specific neutralizing antibody. Science (80-.). 340, 1113-1117 (2013).
52. Kwakkenbos, M. J. et al. Generation of stable monoclonal antibody-producing B cell receptor-positive human memory B cells by genetic programming. Nat. Med. 16, 123-128 (2010).
53. Gilman, M. S. A. A. et al. Characterization of a Prefusion-Specific Antibody That Recognizes a Quaternary, Cleavage-Dependent Epitope on the RSV Fusion Glycoprotein. PLOS Pathog. 11, 1-17 (2015).
54. Corti, D. et al. Cross-neutralization of four paramyxoviruses by a human monoclonal antibody. Nat. 2013 5017467 501, 439-443 (2013).
55. Huang, J., Diaz, D. & Mousa, J. J. Antibody recognition of the Pneumovirus fusion protein trimer interface. bioRxiv 30602, 2020.05.20.107508 (2020).
56. Anderson, L. J., Bingham, P. & Hierholzer, J. C. Neutralization of respiratory syncytial virus by individual and mixtures of F and G protein monoclonal antibodies. J. Virol. 62, 4232 (1988).
57. Kabsch, W. XDS. Acta Crystallogr. Sect. D Biol. Crystallogr. 66, 125 (2010).
58. Emsley, P. & Cowtan, K. Coot: model-building tools for molecular graphics. urn:issn: 0907-4449 60, 2126-2132 (2004).
59. Punjani, A., Rubinstein, J. L., Fleet, D. J. & Brubaker, M. A. cryoSPARC: algorithms for rapid unsupervised cryo-EM structure determination. Nat. Methods 2017 143 14, 290-296 (2017).
60. Sanchez-Garcia, R., Gomez-Blanco, J., Cuervo, A., Carazo, J. M. & Vargas, J. DeepEMhacer: a deep learning solution for cryo-EM volume postprocessing. doi:10.1101/2020.06.12.148296.
61. Goddard, T. D., Huang, C. C. & Ferrin, T. E. Visualizing density maps with UCSF Chimera. J. Struct. Biol. 157, 281-287 (2007).
62. Hsieh, C.- L. et al. Structure-based design of prefusion-stabilized human metapneumovirus fusion proteins. Nat. Commun. 2022 131 13, 1-11 (2022).
63. Jumper, J. et al. Highly accurate protein structure prediction with AlphaFold. Nat. 2021 5967873 596, 583-589 (2021).
64. Mclellan J S, Chen M, Joyce M G, Sastry M, Stewart-Jones G B, Yang Y, Zhang B, Chen L, Srivatsan S, Zheng A, Zhou T, Graepel K W, Kumar A, Moin S, Boyington J C, Chuang G Y, Soto C, Baxa U, Bakker A Q, Spits H, Beaumont T, Zheng Z, Xia N, Ko S Y, Todd J P, Rao S, Graham B S, Kwong P D. Structure-based design of a fusion glycoprotein vaccine for respiratory syncytial virus. Science. 2013 Nov. 1; 342(6158):592-8.

1. A recombinant RSV F protein comprising a first RSV F sequence at least 80%, 85%, 90%, 95%, or 99% identical to positions 26-494 of a second RSV F sequence selected from the group consisting of SEQ ID NOS: 1-4 or positions 26-516 of a third RSV F sequence selected from the group consisting of SEQ ID NOS:5-6, wherein:
the first RSV F sequence comprises 1 or more, 2 or more, 3 or more, 4 or more, 5 or more, 6 or more, 7 or more, 8 or more, 9 or more, 10 or more, or 11 or more of:
a residue other than serine at a position corresponding to position 55 of the second or third RSV F sequence;
a residue other than glutamate at a position corresponding to position 60 of the second or third RSV F sequence;
a residue other than asparagine at a position corresponding to position 70 of the second or third RSV F sequence;
a residue other than glutamine at a position corresponding to position 94 of the second or third RSV F sequence;
a residue other than methionine at a position corresponding to position 97 of the second or third RSV F sequence;
a residue other than serine at a position corresponding to position 128 of the second RSV F sequence or position 150 of the third RSV F sequence;
a residue other than glycine at a position corresponding to position 129 of the second RSV F sequence or position 151 of the third RSV F sequence;
a residue other than asparagine at a position corresponding to position 153 of the second RSV F sequence or position 175 of the third RSV F sequence;
a residue other than glutamine at a position corresponding to position 203 of the second RSV F sequence or position 225 of the third RSV F sequence;
a residue other than asparagine at a position corresponding to position 205 of the second RSV F sequence or position 227 of the third RSV F sequence;
a residue other than valine at a position corresponding to position 256 of the second RSV F sequence or position 278 of the third RSV F sequence;
a residue other than serine at a position corresponding to position 308 of the second RSV F sequence or position 330 of the third RSV F sequence;
a residue other than threonine at a position corresponding to position 315 of the second RSV F sequence or position 337 of the third RSV F sequence;
a residue other than glutamate at a position corresponding to position 356 of the second RSV F sequence or position 378 of the third RSV F sequence;
a residue other than asparagine at a position corresponding to position 358 of the second RSV F sequence or position 380 of the third RSV F sequence;
a residue other than glutamate at a position corresponding to position 465 of the second RSV F sequence or position 487 of the third RSV F sequence;
cysteines at positions corresponding to positions 52 and 128 of the second RSV F sequence or positions 52 and 150 of the third RSV F sequence;
cysteines at positions corresponding to positions 55 and 166 of the second RSV F sequence or positions 55 and 188 of the third RSV F sequence;
cysteines at positions corresponding to positions 60 and 174 of the second RSV F sequence or positions 60 and 196 of the third RSV F sequence;
cysteines at positions corresponding to positions 135 and 161 of the second RSV F sequence or positions 157 and 183 of the third RSV F sequence; and
cysteines at positions corresponding to positions 421 and 444 of the second RSV F sequence or positions 443 and 466 of the third RSV F sequence.

2. The recombinant protein of claim 1, wherein the first RSV F sequence comprises one or more, two or more, three or more, four or more, five or more six or more, or each of:
a residue other than serine at a position corresponding to position 55 of the second or third RSV F sequence;
a residue other than glutamate at a position corresponding to position 60 of the second or third RSV F sequence;
a residue other than serine at a position corresponding to position 128 of the second RSV F sequence or position 150 of the third RSV F sequence;
a residue other than asparagine at a position corresponding to position 153 of the second RSV F sequence or position 175 of the third RSV F sequence;
a residue other than asparagine at a position corresponding to position 205 of the second RSV F sequence or position 227 of the third RSV F sequence;
a residue other than asparagine at a position corresponding to position 358 of the second RSV F sequence or position 380 of the third RSV F sequence; and
a residue other than glutamate at a position corresponding to position 465 of the second RSV F sequence or position 487 of the third RSV F sequence.

3. The recombinant protein of claim 1, wherein, when present:
the residue other than the serine at the position corresponding to position 55 of the second or third RSV F sequence is alanine, isoleucine, leucine, valine, cysteine, or a conservative variant of alanine, isoleucine, leucine, or valine;
the residue other than the glutamate at the position corresponding to position 60 of the second or third RSV F sequence is isoleucine, phenylalanine, cysteine, or a conservative variant of isoleucine or phenylalanine;
the residue other than the asparagine at the position corresponding to position 70 of the second or third RSV F sequence is glutamine or a conservative variant of glutamine;
the residue other than the glutamine at the position corresponding to position 94 of the second or third RSV F sequence is arginine or a conservative variant of arginine;
the residue other than the methionine at the position corresponding to position 97 of the second or third RSV F sequence is isoleucine, threonine, or a conservative variant of isoleucine or threonine;
the residue other than the serine at the position corresponding to position 128 of the second RSV F sequence or position 150 of the third RSV F sequence is glutamate, leucine, cysteine, or a conservative variant of glutamate or leucine;
the residue other than the glycine at the position corresponding to position 129 of the second RSV F sequence or position 151 of the third RSV F sequence is alanine or a conservative variant of alanine;
the residue other than the asparagine at the position corresponding to position 153 of the second RSV F sequence or position 175 of the third RSV F sequence is arginine or a conservative variant of arginine;
the residue other than the glutamine at the position corresponding to position 203 of the second RSV F sequence or position 225 of the third RSV F sequence is glutamate or a conservative variant of glutamate;
the residue other than the asparagine at the position corresponding to position 205 of the second RSV F sequence or position 227 of the third RSV F sequence is leucine, phenylalanine, or a conservative variant of leucine or phenylalanine;
the residue other than the valine at the position corresponding to position 256 of the second RSV F sequence or position 278 of the third RSV F sequence is leucine or a conservative variant of leucine;
the residue other than the serine at the position corresponding to position 308 of the second RSV F sequence or position 330 of the third RSV F sequence is aspartate or a conservative variant of aspartate;
the residue other than the threonine at the position corresponding to position 315 of the second RSV F sequence or position 337 of the third RSV F sequence is leucine or a conservative variant of leucine;
the residue other than the glutamate at the position corresponding to position 356 of the second RSV F sequence or position 378 of the third RSV F sequence is lysine or a conservative variant of lysine;
the residue other than the asparagine at the position corresponding to position 358 of the second RSV F sequence or position 380 of the third RSV F sequence is lysine or a conservative variant of lysine; and
the residue other than the glutamate at the position corresponding to position 465 of the second RSV F sequence or position 487 of the third RSV F sequence is asparagine, valine or a conservative variant of asparagine or valine.

4. The recombinant protein of claim 1, wherein, when present:
the residue other than the serine at the position corresponding to position 55 of the second or third RSV F sequence is alanine, isoleucine, leucine, valine, or cysteine;
the residue other than the glutamate at the position corresponding to position 60 of the second or third RSV F sequence is isoleucine, phenylalanine, or cysteine;
the residue other than the asparagine at the position corresponding to position 70 of the second or third RSV F sequence is glutamine;
the residue other than the glutamine at the position corresponding to position 94 of the second or third RSV F sequence is arginine;
the residue other than the methionine at the position corresponding to position 97 of the second or third RSV F sequence is isoleucine or threonine;
the residue other than the serine at the position corresponding to position 128 of the second RSV F sequence or position 150 of the third RSV F sequence is glutamate, leucine, or cysteine;
the residue other than the glycine at the position corresponding to position 129 of the second RSV F sequence or position 151 of the third RSV F sequence is alanine;
the residue other than the asparagine at the position corresponding to position 153 of the second RSV F sequence or position 175 of the third RSV F sequence is arginine;
the residue other than the glutamine at the position corresponding to position 203 of the second RSV F sequence or position 225 of the third RSV F sequence is glutamate;
the residue other than the asparagine at the position corresponding to position 205 of the second RSV F sequence or position 227 of the third RSV F sequence is leucine or phenylalanine;
the residue other than the valine at the position corresponding to position 256 of the second RSV F sequence or position 278 of the third RSV F sequence is leucine;
the residue other than the serine at the position corresponding to position 308 of the second RSV F sequence or position 330 of the third RSV F sequence is aspartate;
the residue other than the threonine at the position corresponding to position 315 of the second RSV F sequence or position 337 of the third RSV F sequence is leucine;
the residue other than the glutamate at the position corresponding to position 356 of the second RSV F sequence or position 378 of the third RSV F sequence is lysine;
the residue other than the asparagine at the position corresponding to position 358 of the second RSV F sequence or position 380 of the third RSV F sequence is lysine; and
the residue other than the glutamate at the position corresponding to position 465 of the second RSV F sequence or position 487 of the third RSV F sequence is asparagine or valine.

5. The recombinant protein of claim 1, wherein the first RSV F sequence comprises:
phenylalanine or cysteine at the position corresponding to position 60 of the second or third RSV F sequence;
arginine at the position corresponding to position 153 of the second RSV F sequence or position 175 of the third RSV F sequence; and
lysine at the position corresponding to position 358 of the second RSV F sequence or position 380 of the third RSV F sequence.

6. The recombinant protein of claim 1, wherein the first RSV F sequence comprises:
alanine or cysteine at the position corresponding to position 55 of the second or third RSV F sequence;
glutamate or cysteine at the position corresponding to position 128 of the second RSV F sequence or position 150 of the third RSV F sequence;
leucine at the position corresponding to position 205 of the second RSV F sequence or position 227 of the third RSV F sequence; and
asparagine at the position corresponding to position 465 of the second RSV F sequence or position 487 of the third RSV F sequence.

7. The recombinant protein of claim 1, wherein the first RSV F sequence comprises cysteines at positions corresponding to:
positions 52 and 128 of the second RSV F sequence or positions 52 and 150 of the third RSV F sequence;
positions 55 and 166 of the second RSV F sequence or positions 55 and 188 of the third RSV F sequence;
positions 60 and 174 of the second RSV F sequence or positions 60 and 196 of the third RSV F sequence;
positions 135 and 161 of the second RSV F sequence or positions 157 and 183 of the third RSV F sequence;
positions 155 and 268 of the second RSV F sequence or positions 155 and 290 of the third RSF F sequence; and/or
positions 421 and 444 of the second RSV F sequence or positions 443 and 466 of the third RSV F sequence.

8. The recombinant protein of claim 1, wherein the first RSV F sequence comprises cysteines at positions corresponding to:
positions 55 and 166 of the second RSV F sequence or positions 55 and 188 of the third RSV F sequence;
positions 135 and 161 of the second RSV F sequence or positions 157 and 183 of the third RSV F sequence; and
positions 421 and 444 of the second RSV F sequence or positions 443 and 466 of the third RSV F sequence.

9. The recombinant protein of claim 1, wherein the first RSV F sequence is at least 95% or 99% identical to positions 26-494 of the second RSV F sequence and the second RSV F sequence is SEQ ID NO:2.

10. The recombinant protein of claim 1, wherein the protein further comprises a trimerization motif C-terminal to the first RSV F sequence.

11. The recombinant protein of claim 10, wherein the trimerization motif is at least 80%, 85%, 90%, 95%, or 99% identical to a trimerization sequence selected from the group consisting of positions 496-522 of SEQ ID NO:1 (GYIPEAPRDGQAYVRKDGEWVLLSTFL).

12-39. (canceled)

40. A recombinant polynucleotide encoding the protein of claim 1.

41. (canceled)

42. A method of vaccination comprising administering a vaccine comprising the protein of claim 1 or the polynucleotide of claim 40 to a subject in an amount effective to elicit an immune response against the protein.

43. The recombinant protein of claim 9, wherein the first RSV F sequence comprises each of:
a residue other than serine at a position corresponding to position 55 of the second RSV F sequence;
a residue other than glutamate at a position corresponding to position 60 of the second RSV F sequence;
a residue other than serine at a position corresponding to position 128 of the second RSV F sequence;
a residue other than asparagine at a position corresponding to position 153 of the second RSV F sequence;
a residue other than asparagine at a position corresponding to position 205 of the second RSV F sequence;
a residue other than asparagine at a position corresponding to position 358 of the second RSV F sequence; and
a residue other than glutamate at a position corresponding to position 465 of the second RSV F sequence.

44. The recombinant protein of claim 43, wherein:
the residue other than the serine at the position corresponding to position 55 of the second RSV F sequence is alanine, isoleucine, leucine, valine, cysteine, or a conservative variant of alanine, isoleucine, leucine, or valine;
the residue other than the glutamate at the position corresponding to position 60 of the second RSV F sequence is isoleucine, phenylalanine, cysteine, or a conservative variant of isoleucine or phenylalanine;
the residue other than the serine at the position corresponding to position 128 of the second RSV F sequence is glutamate, leucine, cysteine, or a conservative variant of glutamate or leucine;
the residue other than the asparagine at the position corresponding to position 153 of the second RSV F sequence is arginine or a conservative variant of arginine;
the residue other than the asparagine at the position corresponding to position 205 of the second RSV F sequence is leucine, phenylalanine, or a conservative variant of leucine or phenylalanine;
the residue other than the asparagine at the position corresponding to position 358 of the second RSV F sequence is lysine or a conservative variant of lysine; and
the residue other than the glutamate at the position corresponding to position 465 of the second RSV F sequence is asparagine, valine or a conservative variant of asparagine or valine.

45. The recombinant protein of claim 43, wherein:
the residue other than the serine at the position corresponding to position 55 of the second RSV F sequence is alanine, isoleucine, leucine, valine, or cysteine;
the residue other than the glutamate at the position corresponding to position 60 of the second RSV F sequence is isoleucine, phenylalanine, or cysteine;
the residue other than the serine at the position corresponding to position 128 of the second RSV F sequence is glutamate, leucine, or cysteine;
the residue other than the asparagine at the position corresponding to position 153 of the second RSV F sequence is arginine;
the residue other than the asparagine at the position corresponding to position 205 of the second RSV F sequence is leucine or phenylalanine;
the residue other than the asparagine at the position corresponding to position 358 of the second RSV F sequence is lysine; and
the residue other than the glutamate at the position corresponding to position 465 of the second RSV F sequence is asparagine or valine.

46. The recombinant protein of claim 43, wherein the first RSV F sequence comprises:
alanine or cysteine at the position corresponding to position 55 of the second RSV F sequence;
phenylalanine or cysteine at the position corresponding to position 60 of the second RSV F sequence;
glutamate or cysteine at the position corresponding to position 128 of the second RSV F sequence;
arginine at the position corresponding to position 153 of the second RSV F sequence;
leucine at the position corresponding to position 205 of the second RSV F sequence;
lysine at the position corresponding to position 358 of the second RSV F sequence; and
asparagine at the position corresponding to position 465 of the second RSV F sequence.

47. The recombinant protein of claim 43, wherein the first RSV F sequence comprises cysteines at positions corresponding to:
positions 52 and 128 of the second RSV F sequence;
positions 55 and 166 of the second RSV F sequence;
positions 60 and 174 of the second RSV F sequence;
positions 135 and 161 of the second RSV F sequence;
positions 155 and 268 of the second RSV F sequence; and/or
positions 421 and 444 of the second RSV F sequence.

48. The recombinant protein of claim 43, wherein the first RSV F sequence comprises cysteines at positions corresponding to:
positions 55 and 166 of the second RSV F sequence;
positions 135 and 161 of the second RSV F sequence; and
positions 421 and 444 of the second RSV F sequence.
